(19) United States
(12) Patent Application Publication
(10) Pub. No.: US 2025/0099464 A1
(43) Pub. Date: Mar. 27, 2025

(54) PHARMACEUTICAL COMPOSITIONS

(71) Applicant: ViiV Healthcare UK (No.3) Limited, Stevenage (GB)

(72) Inventors: Kevin Sam Chu, Collegeville, PA (US); Clinton Frank Jones, Collegeville, PA (US); Deepak Balkrishna Mundhra, Collegeville, PA (US); Rennan Pan, Collegeville, PA (US); Austin Philip, Collegeville, PA (US); Simone Alidori, Collegeville, PA (US); Manish Kumar Gupta, Collegeville, PA (US); Pratik Saha, Collegeville, PA (US); Pranjal Sameer Taskar, Collegeville, PA (US)

(73) Assignee: ViiV Healthcare UK (No.3) Limited, Stevenage (GB)

(21) Appl. No.: 18/895,498

(22) Filed: Sep. 25, 2024

Related U.S. Application Data
(60) Provisional application No. 63/560,024, filed on Mar. 1, 2024.

(30) Foreign Application Priority Data
Sep. 27, 2023 (WO) PCT/IB2023/059640

(51) Int. Cl. A61K 31/4985; A61K 9/16

(52) U.S. Cl. CPC A61K 31/4985; A61K 9/1623; A61K 9/1652

(57) ABSTRACT

The present invention relates to Human Immunodeficiency Virus (HIV) prevention and treatment. In particular, the invention relates to a pharmaceutical composition comprising: cabotegravir; a wetting agent; a stabilizer; and a tonicity adjuster; wherein cabotegravir is present in the form of particles having a mass median diameter (X50) of between (and including) 2.5 μm and 10 μm.

### CROSS-REFERENCE TO RELATED APPLICATIONS

This application claims the benefit of Provisional Application No. 63/560,024 filed 1 Mar. 2024 and claims the benefit of PCT/IB2023/059640 filed 27 Sep. 2023; each of which are incorporated by reference herein in their entirety.

### SEQUENCE LISTING

The instant application contains a Sequence Listing, which has been submitted electronically in computer readable form in XML file format and is hereby incorporated by reference in its entirety. Said XML file, created on 18 Sep. 2024, is named “70392US02T_Sep_2024” and is 8,151 bytes in size.

### FIELD OF THE INVENTION

The invention relates to Human Immunodeficiency Virus (HIV) treatment or prevention. In particular, the invention relates to long-acting treatment or prevention of HIV.

### BACKGROUND TO THE INVENTION

Patients with HIV infection commonly undergo complex treatment regimens which involve taking multiple pills at regular intervals each day. Patient non-compliance is a known problem accompanying these complex HIV treatment regimens and can lead to the emergence of multiple drug resistant strains of HIV.

The application of long-acting parenteral pharmaceuticals has been established in clinical practice for decades, notably in the areas of contraception, anti-psychotics, and opiate addiction. More recently, long-acting parenteral pharmaceuticals have been proposed as a way of overcoming the non-compliance problem with HIV treatment regimens. Long-acting injectable formulations, some of which are approved and marketed, such as CABENUVA®, have demonstrated prolonged exposures (≥30 days) following injection, enabling dosing at once-monthly and bimonthly intervals.

Achieving an injectable suspension with a high concentration of anti-HIV drug in order to dose less frequently and overcome the non-compliance problem with HIV treatment regimens, whilst keeping the same or similar injection volume as previous HIV treatment regimens in order to maintain patient experience, is desirable. Similarly, where an anti-HIV drug may be used for prevention of HIV (e.g., pre-exposure prophylaxis, or PrEP), achieving an injectable suspension with a high concentration of anti-HIV drug that provides a longer-acting effect could increase adherence due to less frequent dosing. Yet high concentration suspensions typically suffer from difficulty in resuspension and particle size growth. Moreover, high concentration suspensions with larger particle sizes (e.g., on micron scale) are more difficult to stabilize in ready-to-use suspensions.

Lyophilized formulations offer several advantages over ready-to-use suspensions—namely, avoiding resuspension difficulties or failures, maintaining product stability, and overcoming scale-up issues.

There is a need in the art for a long-acting injectable to treat or prevent HIV that can be dosed at longer intervals whilst still achieving the same patient experience, minimizing injection-site reactions, and overcoming known difficulties with high-concentration and/or ready-to-use suspensions.

### SUMMARY OF THE INVENTION

According to a first aspect of the invention, there is provided a pharmaceutical composition comprising:
cabotegravir;
a wetting agent;
a stabilizer; and
a tonicity adjuster;
wherein cabotegravir is present in the form of particles having a mass median diameter (X50) of between (and including) 2.5 μm and 10 μm.

According to a second aspect of the invention, there are provided methods for (a) treating HIV in a human in need thereof comprising administering to said human a therapeutically effective amount of the pharmaceutical composition as defined herein; and (b) preventing HIV in a human comprising administering to said human an effective amount of the pharmaceutical composition as defined herein.

According to a third aspect of the invention, there is provided a pharmaceutical composition as defined herein for use in the treatment or prevention of HIV.

According to a fourth aspect of the invention, there is provided a kit comprising cabotegravir, wherein cabotegravir is present in the form of particles having a mass median diameter (X50) between (and including) 2.5 μm and 10 μm; a wetting agent; a stabilizer; and a tonicity adjuster.

The pharmaceutical compositions of the invention may be advantageous in a number of respects. The pharmaceutical compositions of the invention allow a high concentration of larger-sized cabotegravir particles to be present in the composition. The larger-size particles of the invention, in turn, favorably modify absorption kinetics, thus permitting ultra long-acting therapy, which allows for longer time intervals between dosing compared to existing therapies. This may improve patient compliance, reducing likelihood of drug-resistant HIV strains. Compositions of the invention may also lower injection site reactions therefore improving patient experience. Finally, lyophilized compositions of the invention minimize resuspension failures and maintain product stability and syringeability.

### BRIEF DESCRIPTION OF THE DRAWINGS

FIGS. 1 and 2 are flowcharts for exemplary processes for manufacturing a lyophilized composition as described herein.

FIG. 3 shows a simulation of a median plasma concentration/time plot of a human assigned male at birth who has had an intramuscular loading dose of 3200 mg of cabotegravir (CAB) (a 533 mg/mL formulation of cabotegravir in 3 mL twice—formulation as described herein in cohort 5, example 9) and, 1 month later a first maintenance dose of 1600 mg of cabotegravir and then 1600 mg of cabotegravir every 4 months (a 533 mg/mL formulation of cabotegravir in 3 mL as described herein in cohort 5, example 9). This has been overlaid over the plot of a median plasma concentration over time of a human assigned male at birth who has been administered the approved Cabotegravir 200 mg/mL formulation Q2M (every 2 months) (APRETUDE®—labelled CAB200 in the figure). The black line in the middle of the shaded grey area is the simulated median of the dosing regimen (name CAB-ULA in the figure). The black line in the middle of the grey shaded area is higher than the black dashed line (the median plasma concentration of CAB200). The lower boundary of the grey band (simulated 10th percentile of CAB-ULA) is higher than the lowest dashed line (simulated 10th percentile of CAB200). This thus shows that this regimen maintains CAB plasma concentrations higher than the approved Q2M regimen of CAB200 IM gluteal injections.

FIG. 4 shows a simulation of a median plasma concentration/time plot of a human assigned female at birth who has had an intramuscular loading dose of 3200 mg of cabotegravir (a 533 mg/mL formulation of cabotegravir in 3 mL twice—formulation as described herein in cohort 5, example 9) and, 1 month later a first maintenance dose of 1600 mg of cabotegravir and then 1600 mg of cabotegravir every 4 months (a 533 mg/mL formulation of cabotegravir in 3 mL as described herein in cohort 5, example 9). This has been overlaid over the plot of a median plasma concentration over time of a human assigned female at birth who has been administered the approved Cabotegravir 200 mg/mL formulation Q2M (every 2 months) (APRETUDE®—labelled CAB200 in the figure). The black line in the middle of the shaded grey area is the simulated median of the dosing regimen (name CAB-ULA in the figure). The black line in the middle of the grey shaded area is higher than the black dashed line (the median of CAB200). The lower boundary of the grey band (simulated 10th percentile of CAB-ULA) is higher than the lowest dashed line (simulated 10th percentile of CAB200). This thus shows that this regimen maintains CAB plasma concentrations higher than the approved Q2M regimen of CAB200 IM gluteal injections.

FIG. 5 shows a simulation of a median plasma concentration/time plot of a human assigned male at birth who has had an intramuscular loading dose of 2132 mg cabotegravir (a 533 mg/mL formulation of cabotegravir in 4 mL) and, 1 month later a first maintenance dose of 799.5 mg of cabotegravir and then 799.5 mg of cabotegravir every 4 months (a 533 mg/mL formulation of cabotegravir in 1.5 mL). This has been overlaid over the plot of a median plasma concentration over time of a human assigned male at birth who has been administered approved Cabotegravir 200 mg/mL formulation Q2M. The dotted black line is the median plasma concentration of the approved regimen (APRETUDE®). The grey shaded area shows 10^{th} to 90^{th} percentile plasma concentrations of the simulated regimen. The solid black line is the median of the simulated dosing regimen.

FIG. 6 shows a simulation of a median plasma concentration/time plot of a human assigned female at birth who has had an intramuscular loading dose of 2132 mg cabotegravir (a 533 mg/mL formulation of cabotegravir in 4 mL) and, 1 month later a first maintenance dose of 799.5 mg of cabotegravir and then 799.5 mg of cabotegravir every 4 months (a 533 mg/mL formulation of cabotegravir in 1.5 mL). This has been overlaid over the plot of a median plasma concentration over time of a human assigned female at birth who has been administered approved Cabotegravir 200 mg/mL formulation Q2M. The dotted black line is the median plasma concentration of the approved regimen (APRETUDE®). The grey shaded area shows 10^{th} to 90^{th} percentile plasma concentrations of the simulated regimen. The solid black line is the median of the simulated dosing regimen.

FIG. 7 shows a simulation of a median plasma concentration/time plot of a human assigned male at birth who has had an intramuscular loading dose of cabotegravir (a 400 mg/mL formulation of cabotegravir in 3 mL twice) and, 1 month later a first maintenance dose of 1200 mg of cabotegravir and then 1200 mg of cabotegravir every 4 months (a 400 mg/mL formulation of cabotegravir in 3 mL, see cohort 3, example 9 herein). This has been overlaid over the plot of a median plasma concentration over time of a human assigned male at birth who has been administered approved cabotegravir 200 mg/mL formulation Q2M. The dotted black line in the middle of the shaded grey area is the median plasma concentration of the approved regimen (APRETUDE®). The grey shaded area is the interval of the approved regimen. The solid black line is the median of the simulated dosing regimen and the dotted black lines above and below it are the upper and lower boundaries of the predicted interval of the simulated dosing regimen.

FIG. 8 shows a simulation of a median plasma concentration/time plot of a human assigned female at birth who has had an intramuscular loading dose of cabotegravir (a 400 mg/mL formulation of cabotegravir in 3 mL twice) and, 1 month later a first maintenance dose of 1200 mg of cabotegravir and then 1200 mg of cabotegravir every 4 months (a 400 mg/mL formulation of cabotegravir in 3 mL, see cohort 3, example 9 herein). This has been overlaid over the plot of a median plasma concentration over time of a human assigned female at birth who has been administered approved cabotegravir 200 mg/mL formulation Q2M. The dotted black line in the middle of the shaded grey area is the median plasma concentration of the approved regimen (APRETUDE®). The grey shaded area is the interval of the approved regimen. The solid black line is the median of the simulated dosing regimen and the dotted black lines above and below it are the upper and lower boundaries of the predicted interval of the simulated dosing regimen.

FIG. 9 shows the same simulation as shown in FIG. 3 but overlaid over a dashed line showing 1.05 μg/mL rather than a simulation of the approved regimen. The lower boundary of the grey band (simulated 10th percentile of the dosing regimen shown), labelled as CAB-ULA in the figure, is higher than the horizontal dashed line, i.e., the benchmark for efficacy in people assigned male at birth.

FIG. 10 shows the same simulation as shown in FIG. 4 but overlaid over a dashed line showing 1.39 μg/mL rather than a simulation of the approved regimen. The lower boundary of the grey band (simulated 10th percentile of the dosing regimen shown), labelled as CAB-ULA in the figure, is higher than the horizontal blue dashed line i.e. the benchmark for efficacy for people assigned female at birth.

FIG. 11 shows a simulation of a median plasma concentration/time plot of a human assigned male at birth who has had an intramuscular loading dose of 2132 mg of cabotegravir (533 mg/mL of cabotegravir in 4 mL) and, 1 month later a first maintenance dose of 1066 mg of cabotegravir and then 1066 mg of cabotegravir every 4 months (533 mg/mL of cabotegravir in 2 mL). This has been overlaid over the plot of a median plasma concentration over time of a human assigned male at birth who has been administered approved Cabotegravir 200 mg/mL formulation Q2M. The dotted black is the median plasma concentration of the approved regimen (APRETUDE®). The solid black line is the median of the simulated dosing regimen (labelled Cab-ULA) and the shaded grey area around is it is the predicted interval of the simulated dosing regimen from the 10^{th} to 90^{th} percentile.

FIG. 12 shows a simulation of a median plasma concentration/time plot of a human assigned female at birth who has had an intramuscular loading dose of 2132 mg of cabotegravir (533 mg/mL of cabotegravir in 4 mL) and, 1 month later a first maintenance dose of 1066 mg of cabotegravir and then 1066 mg of cabotegravir every 4 months (533 mg/mL of cabotegravir in 2 mL). This has been overlaid over the plot of a median plasma concentration over time of a human assigned female at birth who has been administered approved Cabotegravir 200 mg/mL formulation Q2M. The dotted black is the median plasma concentration of the approved regimen (APRETUDE®). The solid black line is the median of the simulated dosing regimen (labelled Cab-ULA) and the shaded grey area around is it is the predicted interval of the simulated dosing regimen from the 10^{th} to 90^{th} percentile.

FIG. 13 shows a concentration time course of cabotegravir from intramuscular dosing in rats of two nanosuspension formulations at 200 mg/mL (CAB200, triangles with dashed line) and at 400 mg/mL (CAB400, circles with continuous line), respectively. Data points represent mean±standard deviation.

### DETAILED DESCRIPTION OF THE INVENTION

### Definitions

The term “alkyl” refers to a saturated hydrocarbon radical, straight or branched, having the specified number of carbon atoms. For example, the term “C₁₋₆ alkyl” refers to an alkyl group having 1 to 6 carbon atoms. Exemplary groups include, but are not limited to, methyl, ethyl, propyl (n-propyl and isopropyl), butyl (n-butyl, sec-butyl, isobutyl and tert-butyl), pentyl, and hexyl.

When the term “alkyl” is used in combination with other substituent groups, such as “halo(C₁₋₄)alkyl” and “hydroxy(C₁₋₄)alkyl” the term “alkyl” is intended to encompass a divalent straight or branched chain hydrocarbon radical, wherein the point of attachment is through the alkyl moiety.

The term “alkylene” refers to a divalent radical derived from a straight or branched, saturated hydrocarbon group of, for example, 1 to 3 carbon atoms (C₁₋₃alkylene). Exemplary groups include, but are not limited to, —CH₂—, —CH₂CH₂—, and —CH₂CH₂CH₂—.

The term “cycloalkyl” refers to a non-aromatic, saturated, monocyclic, hydrocarbon ring containing the specified number of carbon atoms. For example, “cycloalkyl” may contain 3 to 8 carbon atoms, i.e., C₃₋₈ cycloalkyl. Exemplary groups include cyclopropyl, cyclobutyl, cyclopentyl, cyclohexyl, cycloheptyl or cyclooctyl.

The term “heteroaryl” refers to a group or moiety comprising an aromatic monovalent monocyclic or bicyclic radical, containing 5 to 10 ring atoms, including at least one heteroatom independently selected from nitrogen, oxygen and sulfur. This term also encompasses bicyclic heterocyclic-aryl compounds containing an aryl ring moiety fused to a heterocycloalkyl ring moiety, containing 5 to 10 ring atoms, including at least one heteroatom independently selected from nitrogen, oxygen and sulfur. Exemplary groups include, but are not limited to furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, triazinyl, benzofuranyl, isobenzofuryl, 2,3-dihydrobenzofuryl, 1,3-benzodioxolyl, dihydrobenzodioxinyl, benzothienyl, indolizinyl, indolyl, isoindolyl, dihydroindolyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, imidazopyridinyl, pyrazolopyridinyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl. Examples of 5-membered “heteroaryl” groups include furanyl, thienyl, pyrrolyl, imidazolyl, pyrazolyl, triazolyl, tetrazolyl, thiazolyl, oxazolyl, isoxazolyl, oxadiazolyl, thiadiazolyl, and isothiazolyl. Examples of 6-membered “heteroaryl” groups include oxo-pyridyl, pyridinyl, pyridazinyl, pyrazinyl, and pyrimidinyl. Examples of 6,6-fused “heteroaryl” groups include quinolinyl, isoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl. Examples of 6,5-fused “heteroaryl” groups include benzofuranyl, benzothienyl, benzimidazolyl, benzthiazolyl, indolizinyl, indolyl, isoindolyl, and indazolyl.

The terms “halogen” and “halo” represent chloro, fluoro, bromo, or iodo substituents.

The term “optionally substituted” indicates that a group may be unsubstituted or substituted with one or more substituents as defined herein. The term “substituted” in reference to a group indicates that a hydrogen atom attached to a member atom within a group is replaced by one of the defined substituents. In the case where groups may be selected from a number of alternative groups, the selected groups may be the same or different.

The term “member atoms” refers to the atom or atoms that form a chain or ring. Where more than one member atom is present in a chain and within a ring, each member atom is covalently bound to an adjacent member atom in the chain or ring. Atoms that make up a substituent group attached to a chain or ring are not member atoms in the chain or ring.

As used herein, the term “aqueous solution” refers to any solution comprising water or in which the solvent is water. Additionally, “aqueous solution” is used to describe solutions displaying commonalities to water or watery solutions, not limited to characteristics such as appearance, smell, color, taste, viscosity, pH, absorbance, or physical state under particular temperatures.

As used herein, the term “lyophilization,” also known as freeze-drying or cryodesiccation, is a dehydration process which involves freezing the product without destroying the physical structure of the matter.

As used herein, the terms “lyophilized” and “freeze-dried” can be used interchangeably herein and refer to a condition and/or state of a sample, formulation, or product obtained by means of lyophilization.

As used interchangeably herein, the terms “lyophilized pharmaceutical composition” and “lyophilized composition” refer to a pharmaceutical composition in lyophilized form, as taught herein, for example a lyophilized powder.

As used herein, the term “pharmaceutically acceptable salts” refers to salts that retain the desired biological activity of the subject compound and exhibit minimal undesired toxicological effects. These pharmaceutically acceptable salts may be prepared in situ during the final isolation and purification of the compound, or by separately reacting the purified compound in its free acid or free base form with a suitable base or acid, respectively.

Pharmaceutically acceptable salts include, amongst others, those described in Berge, J. Pharm. Sci., 1977, 66, 1-19, or those listed in P H Stahl and C G Wermuth, editors, Handbook of Pharmaceutical Salts: Properties, Selection and Use, Second Edition Stahl/Wermuth: Wiley-VCH/VHCA, 2011 (see www(dot)wiley(dot)com/WileyCDA/WileyTitle/productCd-3906390519.html). Suitable pharmaceutically acceptable salts can include acid or base addition salts. Suitable pharmaceutically acceptable salts of the invention include base addition salts.

Representative pharmaceutically acceptable base addition salts include, but are not limited to, aluminium, 2-amino-2-(hydroxymethyl)-1,3-propanediol (TRIS, tromethamine), arginine, benethamine (N-benzylphenethylamine), benzathine (N,N′-dibenzylethylenediamine), bis-(2-hydroxyethyl)amine, bismuth, calcium, chloroprocaine, choline, clemizole (1-p chlorobenzyl-2-pyrrolidine-1′-ylmethylbenzimidazole), cyclohexylamine, dibenzylethylenediamine, diethylamine, diethyltriamine, dimethylamine, dimethylethanolamine, dopamine, ethanolamine, ethylenediamine, L-histidine, iron, isoquinoline, lepidine, lithium, lysine, magnesium, meglumine (N-methylglucamine), piperazine, piperidine, potassium, procaine, quinine, quinoline, sodium, strontium, t-butylamine, and zinc.

As used herein, the term “pharmaceutical composition” means a composition that is suitable for pharmaceutical use.

As used herein, the term “prevention” or “preventing” refers to avoidance of the stated disease in a subject who is not suffering from the stated disease.

As used herein, “reconstitution” refers to the process of restoring a dried, lyophilized, dehydrated, or concentrated matter to its original or liquid state by adding a solvent to the lyophilized matter, allowing the lyophilized matter to rehydrate, followed by agitating the mixture of the solvent and lyophilized matter. The reconstituted matter may be part of a product, formulation, sample, raw material, or any biological material but is certainly not limited to matter falling under the common definition of these terms. Reconstitution can be assessed visually with the naked eye. The lyophilized matter is deemed reconstituted when a homogeneous suspension is observed. In particular, a suspension with a cloudy appearance is considered suitably reconstituted.

As used herein, the term “self-administered” means administration by someone other than a healthcare professional, for example, a patient may administer the pharmaceutical composition to their self or someone else, other than a healthcare professional administering the pharmaceutical composition to the patient.

As used herein, the term “subject” or “patient” refers to a human.

As used herein, the term “treatment” or “treating” refers to alleviating the specified condition, eliminating or reducing the symptoms of the condition, slowing or eliminating the progression, invasion, or spread of the condition, and reducing or delaying the recurrence of the condition in a previously afflicted subject.

As used herein, the term “diameter” refers to a spherical volume equivalent diameter.

As used herein, the term “Cmax” refers to maximum observed plasma concentration.

As used herein, the term “Ctau” refers to trough plasma concentration i.e. the concentration reached immediately before the next dose is administered.

As used herein the term “tmax” refers to time of maximum observed plasma concentration (tmax) of a substance after administration of that substance.

As used herein the term “Area under the curve” or “AUC” refers to area under the concentration (of substance in plasma)—time curve. AUC can be a measure of the integral of the instantaneous concentrations during a time interval and has the units mass*time/volume. AUC is typically calculated by the trapezoidal method (e.g., linear, linear-log). AUC is usually given for the time interval zero to infinity (AUC(0−inf)), and other time intervals are indicated (for example AUC (t1,t2) where t1 and t2 are the starting and finishing times for the interval).

As used herein, the term “about” generally means ±5%, ±10%, ±15%, or ±20% of the numerical value of the number with which it is being used. In an embodiment, the term “about” means ±10% of the numerical value of the number with which it is being used.

As used herein, the term “CD4 binding site (CD4bs) binding protein” refers to antibodies and other protein constructs, such as domains, that are capable of binding to the CD4 binding site of the HIV envelope glycoprotein, gp120. The terms “CD4bs binding protein.” “CD4bs binding domain” and “CD4bs antigen binding protein” are used interchangeably herein. This does not include the natural cognate ligand or receptor. In some embodiments, monoclonal antibodies and antigen binding fragments thereof that bind to the CD4 binding site on gp120 and neutralize HIV-1 are provided herein. In some embodiments, the CD4bs binding protein includes N6 and N6LS, or an antigen binding fragment thereof.

As used herein, the term “antibody” in the broadest sense refers to molecules with an immunoglobulin-like domain (for example IgG, IgM, IgA, IgD or IgE) and includes monoclonal, recombinant, polyclonal, chimeric, human, humanized, multispecific antibodies, including bispecific antibodies, and heteroconjugate antibodies; a single variable domain (e.g., a domain antibody (DAB)), antigen binding antibody fragments, Fab, F(ab′)2, Fv, disulfide linked Fv, single chain Fv, disulfide-linked scFv, diabodies, TANDABS, etc. and modified versions of any of the foregoing (for a summary of alternative “antibody” formats see Holliger and Hudson, Nature Biotechnology, 2005, Vol 23, No. 9, 1126-1136).

As used interchangeably herein, the terms “full antibody or immunoglobulin,” “whole antibody or immunoglobulin,” and “intact antibody or immunoglobulin” refer to a heterotetrameric glycoprotein with an approximate molecular weight of 150,000 Daltons. An intact antibody is composed of two identical heavy chains (HCs) and two identical light chains (LCs) linked by covalent disulfide bonds. This H2L2 structure folds to form three functional domains comprising two antigen-binding fragments, known as ‘Fab’ fragments, and a ‘Fc’ crystallizable fragment. The Fab fragment is composed of the variable domain at the amino-terminus, variable heavy (VH) or variable light (VL), and the constant domain at the carboxyl terminus, CH1 (heavy) and CL (light). The Fc fragment is composed of two domains formed by dimerization of paired CH2 and CH3 regions. The Fc may elicit effector functions by binding to receptors on immune cells or by binding C1q, the first component of the classical complement pathway. The five classes of antibodies IgM, IgA, IgG, IgE and IgD are defined by distinct heavy chain amino acid sequences, which are called μ, α, γ, ε and δ respectively, each heavy chain can pair with either a K or λ light chain. The majority of antibodies in the serum belong to the IgG class, there are four isotypes of human IgG (IgG1, IgG2, IgG3 and IgG4), the sequences of which differ mainly in their hinge region.

Fully human antibodies can be obtained using a variety of methods, for example using yeast-based libraries or transgenic animals (e.g., mice) that are capable of producing repertoires of human antibodies. Yeast presenting human antibodies on their surface that bind to an antigen of interest can be selected using FACS (Fluorescence-Activated Cell Sorting) based methods or by capture on beads using labeled antigens. Transgenic animals that have been modified to express human immunoglobulin genes can be immunized with an antigen of interest and antigen-specific human antibodies isolated using B-cell sorting techniques. Human antibodies produced using these techniques can then be characterized for desired properties such as affinity, developability and selectivity.

Alternative antibody formats include alternative scaffolds in which the one or more CDRs of the antigen binding protein can be arranged onto a suitable non-immunoglobulin protein scaffold or skeleton, such as an affibody, a SpA scaffold, an LDL receptor class A domain, an Avimer (see, e.g., U.S. Patent Application Publication Nos. 2005/0053973, 2005/0089932, 2005/0164301) or an EGF domain.

As used herein, the term “broadly neutralizing antibody” (bnAb) is defined as an antibody which inhibits viral attachment and cell entry via binding to the HIV envelope glycoprotein (Env) (e.g., gp160, gp120, gp41), as a non-limiting example, by a 50% inhibition of infection in vitro, in more than 50%, 60%, 70%, 80%, 90%, 95%, 99% or greater, of a large panel of (greater than 100) HIV-1 envelope pseudotyped viruses and viral isolates. See, e.g., US Published Patent Application No. 20120121597; Burton et al., Broadly Neutralizing Antibodies to HIV and Their Role in Vaccine Design. Annu Rev Immunol. 2016 May 20; 34:635-59.

As used herein, “CDRs” are defined as the complementarity determining region amino acid sequences of an antigen binding protein. These are the hypervariable regions of immunoglobulin heavy and light chains. There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs.

Throughout this specification, amino acid residues in variable domain sequences and variable domain regions within full-length antigen binding sequences, e.g., within an antibody heavy chain sequence or antibody light chain sequence, are numbered according to the Kabat numbering convention. Similarly, the terms “CDR”, “CDRL1”, “CDRL2”, “CDRL3”, “CDRH1”, “CDRH2”, “CDRH3” used in the Examples follow the Kabat numbering convention. For further information, see Kabat et al., Sequences of Proteins of Immunological Interest, 4^{th} Ed., U.S. Department of Health and Human Services, National Institutes of Health (1987).

It will be apparent to those skilled in the art that there are alternative numbering conventions for amino acid residues in variable domain sequences and full-length antibody sequences. There are also alternative numbering conventions for CDR sequences, for example, those set out in Chothia et al. (1989) Nature 342: 877-883. The structure and protein folding of the antigen binding protein may mean that other residues are considered part of the CDR sequence and would be understood to be so by a skilled person.

Other numbering conventions for CDR sequences available to a skilled person include “AbM” (University of Bath) and “contact” (University College London) methods.

Table 1 below represents one definition using each numbering convention for each CDR or binding unit. The Kabat numbering scheme is used in Table 1 to number the variable domain amino acid sequence. It should be noted that some of the CDR definitions may vary depending on the individual publication used.

TABLE 1

| | Kabat CDR | Chothia CDR | AbM CDR | Contact CDR |
|---|---|---|---|---|
| H1 | 31-35/35A/ | 26-32/33/34 | 26-35/35A/35B | 30-35/35A/35B |
| | 35B | | | |
| H2 | 50-65 | 52-56 | 50-58 | 47-58 |
| H3 | 95-102 | 95-102 | 95-102 | 93-101 |
| L1 | 24-34 | 24-34 | 24-34 | 30-36 |
| L2 | 50-56 | 50-56 | 50-56 | 46-55 |
| L3 | 89-97 | 89-97 | 89-97 | 89-96 |

As used herein, “antibody half-life” (or “serum half-life”) refers to the time required for the serum concentration of an antigen binding protein to reach half of its original value. The serum half-life of proteins can be measured by pharmacokinetic studies according to the method described by Kim et al., 1994, Eur. J. of Immuno. 24: 542-548. According to this method, radio-labelled protein is injected intravenously into mice and its plasma concentration is periodically measured as a function of time, for example, at about 3 minutes to about 72 hours after the injection. Other methods for pharmacokinetic analysis and determination of the serum half-life of a molecule will be familiar to those skilled in the art.

Antigen binding proteins of the present invention may have amino acid modifications that increase the affinity of the constant domain or fragment thereof for FcRn. Increasing the serum half-life of therapeutic and diagnostic IgG antibodies and other bioactive molecules has many benefits including reducing the amount and/or frequency of dosing of these molecules. In one embodiment, an antigen binding protein of the invention comprises all or a portion (an FcRn binding portion) of an IgG constant domain having one or more of the following amino acid modifications.

For example, with reference to IgG1, M252Y/S254T/T256E (commonly referred to as “YTE” mutations) and M428LUN434S (commonly referred to as “LS” mutations) increase FcRn binding at pH 6.0 (Wang et al. 2018). Serum half-life can also be enhanced by T250Q/M428L, V2591N308F/M428L, N434A, and T307A/E380A/N434A mutations (with reference to IgG1 and Kabat numbering) (Monnet et al.). Serum half-life and FcRn binding can also be extended by introducing H433K and N434F mutations (commonly referred to as “HN” or “Nhance” mutations) (with reference to IgG1) (WO2006/130834).

### STATEMENT OF THE INVENTION

The present invention provides a pharmaceutical composition comprising:
cabotegravir;
a wetting agent;
a stabilizer; and
a tonicity adjuster;
wherein cabotegravir is present in the form of particles having a mass median diameter (X50) of between (and including) 2.5 μm and 10 μm.

For example, an embodiment of the present invention provides a pharmaceutical composition comprising cabotegravir having a mass median diameter (X50) of between (and including) 2.5 μm and 10 μm; polysorbate 80; sodium CMC; and mannitol.

The pharmaceutical compositions described herein may be administered by any appropriate route. In a preferred embodiment, the compositions are administered parenterally (including subcutaneously, intramuscularly, intravenously, or intradermally). In one embodiment the composition is administered intramuscularly. In another embodiment the composition is administered subcutaneously.

### Cabotegravir

Cabotegravir ((3S,11aR)—N-((2,4-difluorophenyl)methyl)-6-hydroxy-3-methyl-5,7-dioxo-2,3,5,11,11a-hexahydro(1,3)oxazolo(3,2-a)pyrido(1,2-d)pyrazine-8-carboxamide) is described in U.S. Pat. No. 8,129,385 in example Z-9, which example is incorporated herein by reference. Cabotegravir is an integrase strand transfer inhibitor (INSTI) that exhibits subnanomolar potency and antiviral activity against a broad range of HIV-1 strains. Oral administration of cabotegravir has exhibited acceptable safety and tolerability profiles, a long half-life, and few drug-drug interactions. Cabotegravir has been demonstrated to be efficacious in treatment and prevention of HIV both in oral and parenteral dosage forms, see for instance, Margolis D A, Brinson C C, Eron J J, et al. 744 and Rilpivirine as Two Drug Oral Maintenance Therapy: LAI116482 (LATTE) Week 48 Results, 21^{st} Conference on Retroviruses and Opportunistic Infections (CROI); Mar. 3-6, 2014; Boston, MA, Margolis D A, Podzamczer D, Stellbrink H-J, et al. Cabotegravir+Rilpivirine as Long-Acting Maintenance Therapy: LATTE-2 Week 48 Results, 21^{st} International AIDS Conference; Jul. 18-22, 2016; Durban, South Africa, Abstract THAB0206LB. Levin: Conference reports for National AIDS Treatment Advocacy Project (NATAP); 2016, and Markowitz M, Frank I, Grant R, et al. ÈCLAIR: Phase 2A Safety and PK Study of Cabotegravir LA in HIV-Uninfected Men. Abstract presented at 23^{rd} CROI; Feb. 22-25, 2016; Boston, MA. Cabotegravir has been approved by the US FDA for long-acting prevention of HIV infection dosed every two months; and in combination with rilpivirine for long-acting treatment of HIV infection dosed one a month or once every two months.

Cabotegravir is represented by formula (I):

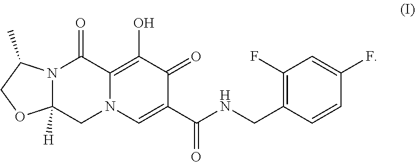 [SMILES: C[C@H]1CO[C@@H]2Cn3cc(C(=O)NCc4ccc(F)cc4F)c(=O)c(O)c3C(=O)N12]

In an embodiment of the invention, cabotegravir is present in the pharmaceutical composition as the free acid.

The pharmaceutical composition of the present invention comprises particles of crystalline cabotegravir. In an embodiment, cabotegravir particles of the pharmaceutical composition have an X50 value greater than or equal to 2.5 μm and less than or equal to 10 μm (i.e., 2.5 μm≤X50≤10 μm). Particle size distribution may be measured by any suitable method, for example, by laser diffraction as described in the Examples section herein.

Without wishing to be bound by theory, it is thought that the larger drug particle size favorably modifies absorption kinetics, thus permitting ultra long-acting therapy. Without wishing to be bound by theory, it is also thought that the larger drug particle size favorably reduces injection site reactions, therefore improving patient experience.

It is further believed that an injectable microsuspension formulation may provide an additional sustained therapeutic effect over commercial nanosuspension formulations due to a reduced drug substance specific surface area resulting in a slower drug depot dissolution. The prolonged dissolution may enable further enhancement to patient compliance and treatment effectiveness when compared to commercial nanosuspension formulations.

Highly potent hydrophobic compounds like cabotegravir may benefit from an increased specific surface area via size reduction to increase drug dissolution in vivo. However, optimization of particle size of these compounds may influence resultant pharmacokinetic performance—smaller crystal sizes of active substances may dissolve more rapidly than larger crystal sizes due to the increased relative surface area. As such, suspensions of larger average particle sizes may exhibit an extended and more controlled release profile from the injected depot when compared to suspensions of smaller average particle sizes. In one embodiment, the pharmaceutical composition has an extended release profile of 1 month or more. In another embodiment, the pharmaceutical composition has an extended release profile of 2 months or more. In another embodiment, the pharmaceutical composition has an extended release profile of 3 months or more. In another embodiment, the pharmaceutical composition has an extended release profile of 4 months or more. In another embodiment, the pharmaceutical composition has an extended release profile of 5 months or more. In another embodiment, the pharmaceutical composition has an extended release profile of 6 months or more.

In an embodiment, the pharmaceutical composition contains about 100 to about 800 mg/mL of cabotegravir. In a further embodiment, the pharmaceutical composition contains about 200 mg/mL to about 700 mg/mL, from about 300 mg/mL to about 650 mg/mL, from about 400 mg/mL to about 600 mg/mL, from about 450 mg/mL to about 600 mg/mL, from about 500 mg/mL to about 600 mg/mL, from about 550 mg/mL to about 600 mg/mL, about 400 mg/mL, about 500 mg/mL, or about 533 mg/mL of cabotegravir. In an embodiment the pharmaceutical composition contains about 400 mg/mL of cabotegravir. In a further embodiment, the pharmaceutical composition contains about 533 mg/mL of cabotegravir.

In an embodiment, the pharmaceutical composition has been reconstituted from a lyophilized powder and contains about 100 to about 800 mg/mL of cabotegravir. In a further embodiment, the pharmaceutical composition contains, after reconstitution, about 200 mg/mL to about 700 mg/mL, from about 300 mg/mL to about 650 mg/mL, from about 400 mg/mL to about 600 mg/mL, about 400, about 500, or about 533 mg/mL of cabotegravir. In an embodiment the pharmaceutical composition contains, after reconstitution, about 400 mg/mL of cabotegravir. In a further embodiment, the pharmaceutical composition contains, after reconstitution, about 533 mg/mL of cabotegravir. In a still further embodiment the pharmaceutical composition contains 533 mg/mL of cabotegravir.

In another embodiment, the pharmaceutical composition contains cabotegravir in an amount between about 300 mg and about 4800 mg. In an embodiment, the pharmaceutical composition contains cabotegravir in an amount between about 350 mg and about 4000 mg. In an embodiment, the pharmaceutical composition contains cabotegravir in an amount between about 375 mg and about 3200 mg. In an embodiment, the pharmaceutical composition contains cabotegravir in an amount between about 400 mg and about 2000 mg. In an embodiment, the pharmaceutical composition contains cabotegravir in an amount of about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg, about 2525 mg, about 2550 mg, about 2575 mg, about 2600 mg, about 2625 mg, about 2650 mg, about 2675 mg, about 2700 mg, about 2725 mg, about 2750 mg, about 2775 mg, about 2800 mg, about 2825 mg, about 2850 mg, about 2875 mg, about 2900 mg, about 2925 mg, about 2950 mg, about 2975 mg, about 3000 mg, about 3025 mg, about 3050 mg, about 3075 mg, about 3100 mg, about 3125 mg, about 3150 mg, about 3175 mg, or about 3200 mg. In an embodiment, the pharmaceutical composition contains cabotegravir in an amount of about 800 mg. In an embodiment, the pharmaceutical composition contains cabotegravir in an amount of about 1600 mg. In an embodiment, the pharmaceutical composition contains cabotegravir in an amount of about 2665 mg. In an embodiment, the pharmaceutical composition contains cabotegravir in an amount of about 3200 mg. In an embodiment, the pharmaceutical composition contains cabotegravir in an amount of about 4000 mg. In an embodiment, the pharmaceutical composition contains cabotegravir in an amount of about 4800 mg.

### Wetting Agents

Wetting agents or surfactants are compounds that, when dissolved in a liquid, can reduce the surface tension of a gas, liquid, or solid surface in that liquid. They are often amphiphilic and may aid in wetting and enhance manufacturability of the drug product. In addition, a surfactant may additionally impart long-term product stability by steric or electrostatic repulsion. Non-ionic surfactants are preferred over ionic surfactants as they are generally non-toxic, non-irritating, and inert. Examples of surfactants include, but are not limited to, polysorbate 20 (Tween-20), polysorbate 80 (Tween-80), sorbitan monolaurate (Span-20), sorbitan monooleate (Span-80), poloxamer 188 (Kolliphor P188), poloxamer 338 (Kolliphor P338), and poloxamer 407 (Kolliphor P407).

In an embodiment, the pharmaceutical composition of the invention comprises polysorbate 80 (PS80) as the wetting agent.

PS80 (IUPAC name: polyoxyethylene (20) sorbitan monooleate; CAS No. 9005-65-6) is a nonionic surfactant and emulsifier derived from polyethoxylated sorbitan and oleic acid. The hydrophilic groups in PS80 are polyethers also known as polyoxyethylene groups, which are polymers of ethylene oxide. In the nomenclature of polysorbates, the numeric designation following “polysorbate” (e.g., “polysorbate 80”) refers to the lipophilic group, in this case, the oleic acid. The structure of PS80 is provided by formula (II):

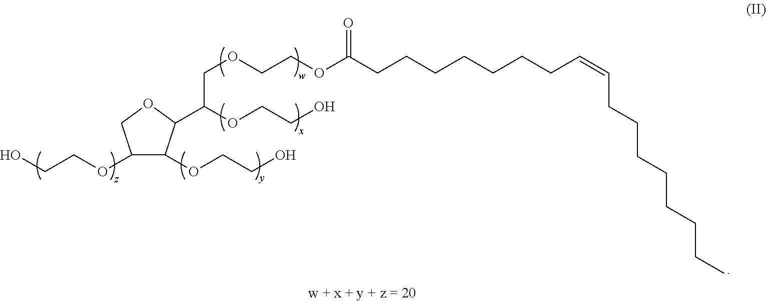

The inventors have surprisingly found that the use of PS80 in a lyophilized microsuspension resulted in superior in vivo performance, tolerability, and increased flexibility in manufacturability.

In an embodiment, the pharmaceutical composition contains from about 0.1 mg/mL to about 150 mg/mL of the wetting agent. In a further embodiment, the pharmaceutical composition contains from about 1 mg/mL to about 80 mg/mL, from about 2 mg/mL to about 40 mg/mL, from about 2.5 mg/mL to about 6 mg/mL, or from about 2.5 mg/mL to about 5 mg/mL of the wetting agent.

In an embodiment, the pharmaceutical composition contains about, in mg/mL, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0 of the wetting agent. In another embodiment, the pharmaceutical composition contains about 3.0 mg/mL of the wetting agent. In another embodiment, the pharmaceutical composition contains about 4.0 mg/mL of the wetting agent. In another embodiment, the pharmaceutical composition contains about 5.3 mg/mL of the wetting agent.

In an embodiment, the pharmaceutical composition has been reconstituted from a lyophilized powder and contains from about 0.1 mg/mL to about 150 mg/mL of the wetting agent. In a further embodiment, the pharmaceutical composition contains, after reconstitution, from about 1 mg/mL to about 80 mg/mL, from about 2 mg/mL to about 40 mg/mL, from about 2.5 mg/mL to about 6 mg/mL, or from about 2.5 mg/mL to about 5 mg/mL of the wetting agent.

In an embodiment, the pharmaceutical composition has been reconstituted from a lyophilized powder and contains about, in mg/mL, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0 of the wetting agent. In another embodiment, the pharmaceutical composition contains, after reconstitution, about 3.0 mg/mL of the wetting agent. In another embodiment, the pharmaceutical composition contains, after reconstitution, about 4.0 mg/mL of the wetting agent. In another embodiment, the pharmaceutical composition contains, after reconstitution, about 5.3 mg/mL of the wetting agent.

In an embodiment, the pharmaceutical composition contains about 0.1 mg to about 900 mg of the wetting agent. In another embodiment, the pharmaceutical composition contains about 0.5 mg to about 200 mg of the wetting agent. In another embodiment, the pharmaceutical composition comprises about 1.0 mg to about 100 mg of the wetting agent. In another embodiment, the pharmaceutical composition contains about 2.0 mg to about 50 mg of the wetting agent. In another embodiment, the pharmaceutical composition contains about, in mg, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.0, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, 28.0, 28.1, 28.2, 28.3, 28.4, 28.5, 28.6, 28.7, 28.8, 28.9, 29.0, 29.1, 29.2, 29.3, 29.4, 29.5, 29.6, 29.7, 29.8, 29.9, 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0, 33.1, 33.2, 33.3, 33.4, 33.5, 33.6, 33.7, 33.8, 33.9, 34.0, 34.1, 34.2, 34.3, 34.4, 34.5, 34.6, 34.7, 34.8, 34.9, 35.0, 35.1, 35.2, 35.3, 35.4, 35.5, 35.6, 35.7, 35.8, 35.9, 36.0, 36.1, 36.2, 36.3, 36.4, 36.5, 36.6, 36.7, 36.8, 36.9, 37.0, 37.1, 37.2, 37.3, 37.4, 37.5, 37.6, 37.7, 37.8, 37.9, 38.0, 38.1, 38.2, 38.3, 38.4, 38.5, 38.6, 38.7, 38.8, 38.9, 39.0, 39.1, 39.2, 39.3, 39.4, 39.5, 39.6, 39.7, 39.8, 39.9, 40.0, 40.1, 40.2, 40.3, 40.4, 40.5, 40.6, 40.7, 40.8, 40.9, 41.0, 41.2, 41.3, 41.4, 41.5, 41.6, 41.7, 41.8, 41.9, 42.0, 42.1, 42.2, 42.3, 42.4, 42.5, 42.6, 42.7, 42.8, 42.9, 43.0, 43.1, 43.2, 43.3, 43.4, 43.5, 43.6, 43.7, 43.8, 43.9, 44.0, 44.1, 44.2, 44.3, 44.4, 44.5, 44.6, 44.7, 44.8, 44.9, 45.0, 45.1, 45.2, 45.3, 45.4, 45.5, 45.6, 45.7, 45.8, 45.9, 46.0, 46.1, 46.2, 46.3, 46.4, 46.5, 46.6, 46.7, 46.8, 46.9, 47.0, 47.1, 47.2, 47.3, 47.4, 47.5, 47.6, 47.7, 47.8, 47.9, 48.0, 48.1, 48.2, 48.3, 48.4, 48.5, 48.6, 48.7, 48.8, 48.9, 49.0, 49.1, 49.2, 49.3, 49.4, 49.5, 49.6, 49.7, 49.8, 49.9, or 50.0 of the wetting agent. In an embodiment, the pharmaceutical composition comprises about 5.9 mg of the wetting agent. In an embodiment, the pharmaceutical composition comprises about 8.0 mg of the wetting agent.

In an embodiment, a weight ratio of the wetting agent to cabotegravir is in a range of from 1:10 to 1:400. In another embodiment, the weight ratio of the wetting agent to cabotegravir is in a range of from 1:50 to 1:200. In another embodiment, the weight ratio of the wetting agent to cabotegravir is in a range of from 1:100 to 1:150. In another embodiment, the weight ratio of the wetting agent to cabotegravir is about 1:100, about 1:101, about 1:102, about 1:103, about 1:104, about 1:105, about 1:106, about 1:107, about 1:108, about 1:109, about 1:110, about 1:111, about 1:112, about 1:113, about 1:114, about 1:115, about 1:116, about 1:117, about 1:118, about 1:119, about 1:120, about 1:121, about 1:122, about 1:123, about 1:124, about 1:125, about 1:126, about 1:127, about 1:128, about 1:129, about 1:130, about 1:131, about 1:132, about 1:133, about 1:134, about 1:135, about 1:136, about 1:137, about 1:138, about 1:139, about 1:140, about 1:141, about 1:142, about 1:143, about 1:144, about 1:145, about 1:146, about 1:147, about 1:148, about 1:149, or about 1:150. In another embodiment, the weight ratio of the wetting agent to cabotegravir is about 1:100. In another embodiment, the weight ratio of the wetting agent to cabotegravir is about 1:105. In another embodiment, the weight ratio of the wetting agent to cabotegravir is about 1:110. In another embodiment, the weight ratio of the wetting agent to cabotegravir is about 1:115. In another embodiment, the weight ratio of the wetting agent to cabotegravir is about 1:136.

### Stabilizers

Stabilizers are components added to help preserve critical product attributes throughout shelf life. In the case of suspensions, stabilizers can be used to induce charge effects, add steric stabilization, increase viscosity of the vehicle, etc. These factors can preserve particle size, product resuspendability, and/or improve manufacturability. Examples of stabilizers include, but are not limited to, sodium carboxymethylcellulose (CMC), polyethylene glycol 3350, polyethylene glycol 4000, povidone K12, and povidone K17.

In an embodiment, the pharmaceutical composition of the invention comprises sodium CMC as the stabilizer.

In an embodiment, the pharmaceutical composition contains about 0.1 to about 150 mg/mL of the stabilizer. In a further embodiment, the pharmaceutical composition contains about 1 mg/mL to about 25 mg/mL, from about 2 mg/mL to about 15 mg/mL, from about 2 mg/mL to about 10 mg/mL, or from about 3 mg/mL to about 10 mg/mL of the stabilizer.

In an embodiment, the pharmaceutical composition contains about, in mg/mL, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 of the stabilizer. In another embodiment, the pharmaceutical composition contains about 3.7 mg/mL of the stabilizer. In another embodiment, the pharmaceutical composition contains about 5.0 mg/mL of the stabilizer. In another embodiment, the pharmaceutical composition contains about 6.7 mg/mL of the stabilizer.

In an embodiment, the pharmaceutical composition has been reconstituted from a lyophilized powder and contains about 0.1 to about 150 mg/mL of the stabilizer. In a further embodiment, the pharmaceutical composition contains, after reconstitution, about 1 mg/mL to about 25 mg/mL, from about 2 mg/mL to about 15 mg/mL, from about 2 mg/mL to about 10 mg/mL, or from about 3 mg/mL to about 10 mg/mL of the stabilizer.

In an embodiment, the pharmaceutical composition has been reconstituted from a lyophilized powder and contains about, in mg/mL, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 of the stabilizer. In another embodiment, the pharmaceutical composition contains, after reconstitution, about 3.7 mg/mL of the stabilizer. In another embodiment, the pharmaceutical composition contains, after reconstitution, about 5.0 mg/mL of the stabilizer. In another embodiment, the pharmaceutical composition contains, after reconstitution, about 6.7 mg/mL of the stabilizer.

In an embodiment, the pharmaceutical composition contains about 0.1 mg to about 300 mg of the stabilizer. In another embodiment, the pharmaceutical composition contains about 1.0 mg to about 200 mg of the stabilizer. In another embodiment, the pharmaceutical composition comprises about 2.0 mg to about 100 mg of the stabilizer. In another embodiment, the pharmaceutical composition contains about 4.0 mg to about 50 mg of the stabilizer. In another embodiment, the pharmaceutical composition contains about, in mg, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.0, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, 28.0, 28.1, 28.2, 28.3, 28.4, 28.5, 28.6, 28.7, 28.8, 28.9, 29.0, 29.1, 29.2, 29.3, 29.4, 29.5, 29.6, 29.7, 29.8, 29.9, 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0, 33.1, 33.2, 33.3, 33.4, 33.5, 33.6, 33.7, 33.8, 33.9, 34.0, 34.1, 34.2, 34.3, 34.4, 34.5, 34.6, 34.7, 34.8, 34.9, 35.0, 35.1, 35.2, 35.3, 35.4, 35.5, 35.6, 35.7, 35.8, 35.9, 36.0, 36.1, 36.2, 36.3, 36.4, 36.5, 36.6, 36.7, 36.8, 36.9, 37.0, 37.1, 37.2, 37.3, 37.4, 37.5, 37.6, 37.7, 37.8, 37.9, 38.0, 38.1, 38.2, 38.3, 38.4, 38.5, 38.6, 38.7, 38.8, 38.9, 39.0, 39.1, 39.2, 39.3, 39.4, 39.5, 39.6, 39.7, 39.8, 39.9, 40.0, 40.1, 40.2, 40.3, 40.4, 40.5, 40.6, 40.7, 40.8, 40.9, 41.0, 41.2, 41.3, 41.4, 41.5, 41.6, 41.7, 41.8, 41.9, 42.0, 42.1, 42.2, 42.3, 42.4, 42.5, 42.6, 42.7, 42.8, 42.9, 43.0, 43.1, 43.2, 43.3, 43.4, 43.5, 43.6, 43.7, 43.8, 43.9, 44.0, 44.1, 44.2, 44.3, 44.4, 44.5, 44.6, 44.7, 44.8, 44.9, 45.0, 45.1, 45.2, 45.3, 45.4, 45.5, 45.6, 45.7, 45.8, 45.9, 46.0, 46.1, 46.2, 46.3, 46.4, 46.5, 46.6, 46.7, 46.8, 46.9, 47.0, 47.1, 47.2, 47.3, 47.4, 47.5, 47.6, 47.7, 47.8, 47.9, 48.0, 48.1, 48.2, 48.3, 48.4, 48.5, 48.6, 48.7, 48.8, 48.9, 49.0, 49.1, 49.2, 49.3, 49.4, 49.5, 49.6, 49.7, 49.8, 49.9, or 50.0 of the stabilizer. In an embodiment, the pharmaceutical composition comprises about 7.4 mg of the stabilizer. In an embodiment, the pharmaceutical composition comprises about 10.0 mg of the stabilizer.

In an embodiment, a weight ratio of the stabilizer to cabotegravir is in a range of from 1:10 to 1:400. In another embodiment, the weight ratio of the stabilizer to cabotegravir is in a range of from 1:40 to 1:200. In another embodiment, the weight ratio of the stabilizer to cabotegravir is in a range of from 1:70 to 1:120. In another embodiment, the weight ratio of the stabilizer to cabotegravir is about 1:70, about 1:71, about 1:72, about 1:73, about 1:74, about 1:75, about 1:76, about 1:77, about 1:78, about 1:79, about 1:80, about 1:81, about 1:82, about 1:83, about 1:84, about 1:85, about 1:86, about 1:87, about 1:88, about 1:89, about 1:90, about 1:91, about 1:92, about 1:93, about 1:94, about 1:95, about 1:96, about 1:97, about 1:98, about 1:99, about 1:100, about 1:101, about 1:102, about 1:103, about 1:104, about 1:105, about 1:106, about 1:107, about 1:108, about 1:109, about 1:110, about 1:111, about 1:112, about 1:113, about 1:114, about 1:115, about 1:116, about 1:117, about 1:118, about 1:119, or about 1:120. In another embodiment, the weight ratio of the stabilizer to cabotegravir is about 1:80. In another embodiment, the weight ratio of the stabilizer to cabotegravir is about 1:100. In another embodiment, the weight ratio of the stabilizer to cabotegravir is about 1:101. In another embodiment, the weight ratio of the stabilizer to cabotegravir is about 1:102. In another embodiment, the weight ratio of the stabilizer to cabotegravir is about 1:103. In another embodiment, the weight ratio of the stabilizer to cabotegravir is about 1:104. In another embodiment, the weight ratio of the stabilizer to cabotegravir is about 1:105. In another embodiment, the weight ratio of the stabilizer to cabotegravir is about 1:106. In another embodiment, the weight ratio of the stabilizer to cabotegravir is about 1:107. In another embodiment, the weight ratio of the stabilizer to cabotegravir is about 1:108.

### Tonicity Adjusters

Tonicity adjusters act to provide and maintain a stable tonicity for the pharmaceutical composition disclosed herein. In some embodiments, tonicity adjusters also function as a non-aqueous solvent, a solubilizer, and/or a stabilizer. In such instances, tonicity adjusters may be used at concentrations higher than needed for tonicity if their primary purpose is stabilization or may be used at concentrations higher than needed for stabilization if their primary purpose is tonicity adjustment.

In some embodiments, the tonicity adjuster is a pharmaceutically acceptable inorganic chloride, e.g., potassium chloride, sodium chloride, magnesium chloride or calcium chloride. In yet other embodiments, the tonicity adjuster is a saccharide such as mannitol, sorbitol, lactose, trehalose, raffinose, dextrose, maltose, galactose, sucrose, or polysucrose. In still further aspects, the tonicity adjuster is mannitol. In other aspects, the tonicity adjuster is a non-aqueous polar aprotic or protic materials such as polyethylene glycol, N,N-dimethylacetamide, N-methyl pyrrolidone, glycerol, propylene glycol, ethanol, t-butyl alcohol, benzyl alcohol, benzyl benzoate, dimethyl sulfoxide, or glycerol. In further aspects, the tonicity adjuster is a polymer such as polyethylene glycol, polygalacturonic acid, galacturonic acid, polyvinylpyrrolidine (PVP), for example, PEG 300, PEG 400, PEG 3350, PEG 6000, or PEG 8000. In still other aspects, the tonicity adjuster is an amino acid such as lysine, arginine, glycine, methionine, or other amino acids. In yet further aspects, the tonicity adjuster is a cyclodextrin such as dextran, Ficoll, and polyvinylpyrrolidone, and other similar excipients and combinations of these agents.

In an embodiment, the pharmaceutical composition of the invention comprises mannitol as the tonicity adjuster.

In an embodiment, the pharmaceutical composition contains about 0.1 to about 250 mg/mL of the tonicity adjuster. In a further embodiment, the pharmaceutical composition contains about 1 mg/mL to about 150 mg/mL, from about 10 mg/mL to about 125 mg/mL, from about 15 mg/mL to about 60 mg/mL, from about 15 mg/mL to about 50 mg/mL, from about 20 mg/mL to about 50 mg/mL, or from about 20 mg/mL to about 40 mg/mL of the tonicity adjuster.

In an embodiment, the pharmaceutical composition contains about, in mg/mL, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.0, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, 28.0, 28.1, 28.2, 28.3, 28.4, 28.5, 28.6, 28.7, 28.8, 28.9, 29.0, 29.1, 29.2, 29.3, 29.4, 29.5, 29.6, 29.7, 29.8, 29.9, 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0, 33.1, 33.2, 33.3, 33.4, 33.5, 33.6, 33.7, 33.8, 33.9, 34.0, 34.1, 34.2, 34.3, 34.4, 34.5, 34.6, 34.7, 34.8, 34.9, 35.0, 35.1, 35.2, 35.3, 35.4, 35.5, 35.6, 35.7, 35.8, 35.9, 36.0, 36.1, 36.2, 36.3, 36.4, 36.5, 36.6, 36.7, 36.8, 36.9, 37.0, 37.1, 37.2, 37.3, 37.4, 37.5, 37.6, 37.7, 37.8, 37.9, 38.0, 38.1, 38.2, 38.3, 38.4, 38.5, 38.6, 38.7, 38.8, 38.9, 39.0, 39.1, 39.2, 39.3, 39.4, 39.5, 39.6, 39.7, 39.8, 39.9, 40.0, 40.1, 40.2, 40.3, 40.4, 40.5, 40.6, 40.7, 40.8, 40.9, 41.0, 41.1, 41.2, 41.3, 41.4, 41.5, 41.6, 41.7, 41.8, 41.9, 42.0, 42.1, 42.2, 42.3, 42.4, 42.5, 42.6, 42.7, 42.8, 42.9, 43.0, 43.1, 43.2, 43.3, 43.4, 43.5, 43.6, 43.7, 43.8, 43.9, 44.0, 44.1, 44.2, 44.3, 44.4, 44.5, 44.6, 44.7, 44.8, 44.9, 45.0, 45.1, 45.2, 45.3, 45.4, 45.5, 45.6, 45.7, 45.8, 45.9, 46.0, 46.1, 46.2, 46.3, 46.4, 46.5, 46.6, 46.7, 46.8, 46.9, 47.0, 47.1, 47.2, 47.3, 47.4, 47.5, 47.6, 47.7, 47.8, 47.9, 48.0, 48.1, 48.2, 48.3, 48.4, 48.5, 48.6, 48.7, 48.8, 48.9, 49.0, 49.1, 49.2, 49.3, 49.4, 49.5, 49.6, 49.7, 49.8, 49.9, or 50.0 of the tonicity adjuster. In another embodiment, the pharmaceutical composition contains about 25.9 mg/mL of the tonicity adjuster. In another embodiment, the pharmaceutical composition contains about 35.0 mg/mL of the tonicity adjuster. In another embodiment, the pharmaceutical composition contains about 46.6 mg/mL of the tonicity adjuster.

In an embodiment, the pharmaceutical composition has been reconstituted from a lyophilized powder and contains about 0.1 to about 250 mg/mL of the tonicity adjuster. In a further embodiment, the pharmaceutical composition contains, after reconstitution, about 1 mg/mL to about 150 mg/mL, from about 10 mg/mL to about 125 mg/mL, from about 15 mg/mL to about 60 mg/mL, from about 15 mg/mL to about 50 mg/mL, from about 20 mg/mL to about 50 mg/mL, or from about 20 mg/mL to about 40 mg/mL of the tonicity adjuster.

In an embodiment, the pharmaceutical composition has been reconstituted from a lyophilized powder and contains about, in mg/mL, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.0, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, 28.0, 28.1, 28.2, 28.3, 28.4, 28.5, 28.6, 28.7, 28.8, 28.9, 29.0, 29.1, 29.2, 29.3, 29.4, 29.5, 29.6, 29.7, 29.8, 29.9, 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0, 33.1, 33.2, 33.3, 33.4, 33.5, 33.6, 33.7, 33.8, 33.9, 34.0, 34.1, 34.2, 34.3, 34.4, 34.5, 34.6, 34.7, 34.8, 34.9, 35.0, 35.1, 35.2, 35.3, 35.4, 35.5, 35.6, 35.7, 35.8, 35.9, 36.0, 36.1, 36.2, 36.3, 36.4, 36.5, 36.6, 36.7, 36.8, 36.9, 37.0, 37.1, 37.2, 37.3, 37.4, 37.5, 37.6, 37.7, 37.8, 37.9, 38.0, 38.1, 38.2, 38.3, 38.4, 38.5, 38.6, 38.7, 38.8, 38.9, 39.0, 39.1, 39.2, 39.3, 39.4, 39.5, 39.6, 39.7, 39.8, 39.9, 40.0, 40.1, 40.2, 40.3, 40.4, 40.5, 40.6, 40.7, 40.8, 40.9, 41.0, 41.1, 41.2, 41.3, 41.4, 41.5, 41.6, 41.7, 41.8, 41.9, 42.0, 42.1, 42.2, 42.3, 42.4, 42.5, 42.6, 42.7, 42.8, 42.9, 43.0, 43.1, 43.2, 43.3, 43.4, 43.5, 43.6, 43.7, 43.8, 43.9, 44.0, 44.1, 44.2, 44.3, 44.4, 44.5, 44.6, 44.7, 44.8, 44.9, 45.0, 45.1, 45.2, 45.3, 45.4, 45.5, 45.6, 45.7, 45.8, 45.9, 46.0, 46.1, 46.2, 46.3, 46.4, 46.5, 46.6, 46.7, 46.8, 46.9, 47.0, 47.1, 47.2, 47.3, 47.4, 47.5, 47.6, 47.7, 47.8, 47.9, 48.0, 48.1, 48.2, 48.3, 48.4, 48.5, 48.6, 48.7, 48.8, 48.9, 49.0, 49.1, 49.2, 49.3, 49.4, 49.5, 49.6, 49.7, 49.8, 49.9, or 50.0 of the tonicity adjuster. In another embodiment, the pharmaceutical composition contains, after reconstitution, about 25.9 mg/mL of the tonicity adjuster. In another embodiment, the pharmaceutical composition contains, after reconstitution, about 35.0 mg/mL of the tonicity adjuster. In another embodiment, the pharmaceutical composition contains, after reconstitution, about 46.6 mg/mL of the tonicity adjuster.

In an embodiment, the pharmaceutical composition contains about 0.1 mg to about 400 mg of the tonicity adjuster. In another embodiment, the pharmaceutical composition contains about 1.0 mg to about 300 mg of the tonicity adjuster. In another embodiment, the pharmaceutical composition comprises about 10 mg to about 100 mg of the tonicity adjuster. In another embodiment, the pharmaceutical composition contains about 35 mg to about 80 mg of the tonicity adjuster. In another embodiment, the pharmaceutical composition contains about, in mg, 35.0, 35.1, 35.2, 35.3, 35.4, 35.5, 35.6, 35.7, 35.8, 35.9, 36.0, 36.1, 36.2, 36.3, 36.4, 36.5, 36.6, 36.7, 36.8, 36.9, 37.0, 37.1, 37.2, 37.3, 37.4, 37.5, 37.6, 37.7, 37.8, 37.9, 38.0, 38.1, 38.2, 38.3, 38.4, 38.5, 38.6, 38.7, 38.8, 38.9, 39.0, 39.1, 39.2, 39.3, 39.4, 39.5, 39.6, 39.7, 39.8, 39.9, 40.0, 40.1, 40.2, 40.3, 40.4, 40.5, 40.6, 40.7, 40.8, 40.9, 41.0, 41.1, 41.2, 41.3, 41.4, 41.5, 41.6, 41.7, 41.8, 41.9, 42.0, 42.1, 42.2, 42.3, 42.4, 42.5, 42.6, 42.7, 42.8, 42.9, 43.0, 43.1, 43.2, 43.3, 43.4, 43.5, 43.6, 43.7, 43.8, 43.9, 44.0, 44.1, 44.2, 44.3, 44.4, 44.5, 44.6, 44.7, 44.8, 44.9, 45.0, 45.1, 45.2, 45.3, 45.4, 45.5, 45.6, 45.7, 45.8, 45.9, 46.0, 46.1, 46.2, 46.3, 46.4, 46.5, 46.6, 46.7, 46.8, 46.9, 47.0, 47.1, 47.2, 47.3, 47.4, 47.5, 47.6, 47.7, 47.8, 47.9, 48.0, 48.1, 48.2, 48.3, 48.4, 48.5, 48.6, 48.7, 48.8, 48.9, 49.0, 49.1, 49.2, 49.3, 49.4, 49.5, 49.6, 49.7, 49.8, 49.9, 50.0, 50.1, 50.2, 50.3, 50.4, 50.5, 50.6, 50.7, 50.8, 50.9, 51.0, 51.1, 51.2, 51.3, 51.4, 51.5, 51.6, 51.7, 51.8, 51.9, 52.0, 52.1, 52.2, 52.3, 52.4, 52.5, 52.6, 52.7, 52.8, 52.9, 53.0, 53.1, 53.2, 53.3, 53.4, 53.5, 53.6, 53.7, 53.8, 53.9, 54.0, 54.1, 54.2, 54.3, 54.4, 54.5, 54.6, 54.7, 54.8, 54.9, 55.0, 55.1, 55.2, 55.3, 55.4, 55.5, 55.6, 55.7, 55.8, 55.9, 56.0, 56.1, 56.2, 56.3, 56.4, 56.5, 56.6, 56.7, 56.8, 56.9, 57.0, 57.1, 57.2, 57.3, 57.4, 57.5, 57.6, 57.7, 57.8, 57.9, 58.0, 58.1, 58.2, 58.3, 58.4, 58.5, 58.6, 58.7, 58.8, 58.9, 59.0, 59.1, 59.2, 59.3, 59.4, 59.5, 59.6, 59.7, 59.8, 59.9, 60.0, 60.1, 60.2, 60.3, 60.4, 60.5, 60.6, 60.7, 60.8, 60.9, 61.0, 61.1, 61.2, 61.3, 61.4, 61.5, 61.6, 61.7, 61.8, 61.9, 62.0, 62.1, 62.2, 62.3, 62.4, 62.5, 62.6, 62.7, 62.8, 62.9, 63.0, 63.1, 63.2, 63.3, 63.4, 63.5, 63.6, 63.7, 63.8, 63.9, 64.0, 64.1, 64.2, 64.3, 64.4, 64.5, 64.6, 64.7, 64.8, 64.9, 65.0, 65.1, 65.2, 65.3, 65.4, 65.5, 65.6, 65.7, 65.8, 65.9, 66.0, 66.1, 66.2, 66.3, 66.4, 66.5, 66.6, 66.7, 66.8, 66.9, 67.0, 67.1, 67.2, 67.3, 67.4, 67.5, 67.6, 67.7, 67.8, 67.9, 68.0, 68.1, 68.2, 68.3, 68.4, 68.5, 68.6, 68.7, 68.8, 68.9, 69.0, 69.1, 69.2, 69.3, 69.4, 69.5, 69.6, 69.7, 69.8, 69.9, 70.0, 70.1, 70.2, 70.3, 70.4, 70.5, 70.6, 70.7, 70.8, 70.9, 71.0, 71.1, 71.2, 71.3, 71.4, 71.5, 71.6, 71.7, 71.8, 71.9, 72.0, 72.1, 72.2, 72.3, 72.4, 72.5, 72.6, 72.7, 72.8, 72.9, 73.0, 73.1, 73.2, 73.3, 73.4, 73.5, 73.6, 73.7, 73.8, 73.9, 74.0, 74.1, 74.2, 74.3, 74.4, 74.5, 74.6, 74.7, 74.8, 74.9, 75.0, 75.1, 75.2, 75.3, 75.4, 75.5, 75.6, 75.7, 75.8, 75.9, 76.0, 76.1, 76.2, 76.3, 76.4, 76.5, 76.6, 76.7, 76.8, 76.9, 77.0, 77.1, 77.2, 77.3, 77.4, 77.5, 77.6, 77.7, 77.8, 77.9, 78.0, 78.1, 78.2, 78.3, 78.4, 78.5, 78.6, 78.7, 78.8, 78.9, 79.0, 79.1, 79.2, 79.3, 79.4, 79.5, 79.6, 79.7, 79.8, 79.9, or 80.0 of the tonicity adjuster. In an embodiment, the pharmaceutical composition comprises about 51.8 mg of the tonicity adjuster. In an embodiment, the pharmaceutical composition comprises about 70 mg of the tonicity adjuster. In an embodiment, the pharmaceutical composition comprises about 105 mg of the tonicity adjuster. In an embodiment, the pharmaceutical composition comprises about 140 mg of the tonicity adjuster. In an embodiment, the pharmaceutical composition comprises about 175 mg of the tonicity adjuster. In an embodiment, the pharmaceutical composition comprises about 210 mg of the tonicity adjuster. In an embodiment, the pharmaceutical composition comprises about 245 mg of the tonicity adjuster. In an embodiment, the pharmaceutical composition comprises about 280 mg of the tonicity adjuster.

In an embodiment, a weight ratio of the tonicity adjuster to cabotegravir is in a range of from 1:1 to 1:100. In another embodiment, the weight ratio of the tonicity adjuster to cabotegravir is in a range of from 1:5 to 1:50. In another embodiment, the weight ratio of the tonicity adjuster to cabotegravir is in a range of from 1:8 to 1:25. In another embodiment, the weight ratio of the tonicity adjuster to cabotegravir is about 1:8, about 1:9, about 1:10, about 1:11, about 1:12, about 1:13, about 1:14, about 1:15, about 1:16, about 1:17, about 1:18, about 1:19, about 1:20, about 1:21, about 1:22, about 1:23, about 1:24, or about 1:25. In another embodiment, the weight ratio of the tonicity adjuster to cabotegravir is about 1:8. In another embodiment, the weight ratio of the tonicity adjuster to cabotegravir is about 1:9. In another embodiment, the weight ratio of the tonicity adjuster to cabotegravir is about 1:10. In another embodiment, the weight ratio of the tonicity adjuster to cabotegravir is about 1:11. In another embodiment, the weight ratio of the tonicity adjuster to cabotegravir is about 1:12. In another embodiment, the weight ratio of the tonicity adjuster to cabotegravir is about 1:13. In another embodiment, the weight ratio of the tonicity adjuster to cabotegravir is about 1:14. In another embodiment, the weight ratio of the tonicity adjuster to cabotegravir is about 1:15.

### Cabotegravir Particle Size

Dissolution properties of the pharmaceutical composition are affected, inter alia, by particle size and particle size distribution of the active pharmaceutical ingredient (i.e., cabotegravir).

As used herein, X50 (or “the X50 value”) is the cabotegravir particle diameter, in microns, at which 50% by volume of the cabotegravir particles have a smaller diameter and 50% by volume have a larger diameter, also known as the mass median diameter (MMD) or the median of the particle size distribution by volume.

As used herein, X90 (or “the X90 value”) is the cabotegravir particle diameter, in microns, at which 90% by volume of the cabotegravir particles have a smaller diameter and 10% by volume have a larger diameter.

As used herein, X10 (or “the X10 value”) is the cabotegravir particle diameter, in microns, at which 10% by volume of the cabotegravir particles have a smaller diameter and 90% by volume have a larger diameter.

In an embodiment, the pharmaceutical composition has a particle size distribution by volume such that 90% of the cabotegravir particles have a particle diameter less than or equal to 25 μm (i.e., X90 is 25 μm). In an embodiment, cabotegravir particles of the pharmaceutical composition have an X90 value greater than or equal to 5 μm and less than or equal to 25 μm (i.e., 5 μm s X90 s 25 μm). In an embodiment, the pharmaceutical composition has a particle size distribution by volume such that 90% of the cabotegravir particles have a particle diameter less than or equal to 20 μm (i.e., X90 is 20 μm). In an embodiment, cabotegravir particles of the pharmaceutical composition have an X90 value greater than or equal to 6 μm and less than or equal to 20 μm (i.e., 6 μm s X90 s 20 μm). In an embodiment, the pharmaceutical composition has a particle size distribution by volume such that 90% of the cabotegravir particles have a particle diameter less than or equal to 18 μm (i.e., X90 is 18 μm). In an embodiment, cabotegravir particles of the pharmaceutical composition have an X90 value greater than or equal to 7 μm and less than or equal to 18 μm (i.e., 7 μm s X90 s 18 μm).

In an embodiment, the pharmaceutical composition has a particle size distribution by volume such that 90% of the cabotegravir particles (X90) have a particle diameter smaller than or equal to 7.0 μm, 7.1 μm, 7.2 μm, 7.3 μm, 7.4 μm, 7.5 μm, 7.6 μm, 7.7 μm, 7.8 μm, 7.9 μm, 8.0 μm, 8.1 μm, 8.2 μm, 8.3 μm, 8.4 μm, 8.5 μm, 8.6 μm, 8.7 μm, 8.8 μm, 8.9 μm, 9.0 μm, 9.1 μm, 9.2 μm, 9.3 μm, 9.4 μm, 9.5 μm, 9.6 μm, 9.7 μm, 9.8 μm, 9.9 μm, 10.0 μm, 10.1 μm, 10.2 μm, 10.3 μm, 10.4 μm, 10.5 μm, 10.6 μm, 10.7 μm, 10.8 μm, 10.9 μm, 11.0 μm, 11.1 μm, 11.2 μm, 11.3 μm, 11.4 μm, 11.5 μm, 11.6 μm, 11.7 μm, 11.8 μm, 11.9 μm, 12.0 μm, 12.1 μm, 12.2 μm, 12.3 μm, 12.4 μm, 12.5 μm, 12.6 μm, 12.7 μm, 12.8 μm, 12.9 μm, 13.0 μm, 13.1 μm, 13.2 μm, 13.3 μm, 13.4 μm, 13.5 μm, 13.6 μm, 13.7 μm, 13.8 μm, 13.9 μm, 14.0 μm, 14.1 μm, 14.2 μm, 14.3 μm, 14.4 μm, 14.5 μm, 14.6 μm, 14.7 μm, 14.8 μm, 14.9 μm, 15.0 μm, 15.1 μm, 15.2 μm, 15.3 μm, 15.4 μm, 15.5 μm, 15.6 μm, 15.7 μm, 15.8 μm, 15.9 μm, 16.0 μm, 16.1 μm, 16.2 μm, 16.3 μm, 16.4 μm, 16.5 μm, 16.6 μm, 16.7 μm, 16.8 μm, 16.9 μm, 17.0 μm, 17.1 μm, 17.2 μm, 17.3 μm, 17.4 μm, 17.5 μm, 17.6 μm, 17.7 μm, 17.8 μm, 17.9 μm, or 18.0 μm. In another embodiment, the pharmaceutical composition has a particle size distribution by volume such that 90% of the cabotegravir particles (X90) have a particle diameter smaller than or equal to 9 μm (i.e., X90 is 9 μm). In another embodiment, the pharmaceutical composition has a particle size distribution by volume such that 90% of the cabotegravir particles (X90) have a particle diameter smaller than or equal to 14 μm (i.e., X90 is 14 μm). In another embodiment, the pharmaceutical composition has a particle size distribution by volume such that 90% of the cabotegravir particles (X90) have a particle diameter smaller than or equal to 17 μm (i.e., X90 is 17 μm).

In an embodiment, the pharmaceutical composition has a particle size distribution by volume such that 50% of the cabotegravir particles have a particle diameter less than or equal to 10 μm (i.e., X50 is 10 μm). In an embodiment, cabotegravir particles of the pharmaceutical composition have an X50 value greater than or equal to 2.5 μm and less than or equal to 10 μm (i.e., 2.5 μm s X50 s 10 μm). In an embodiment, the pharmaceutical composition has a particle size distribution by volume such that 50% of the cabotegravir particles have a particle diameter less than or equal to 8.5 μm (i.e., X50 is 8.5 μm). In an embodiment, cabotegravir particles of the pharmaceutical composition have an X50 value greater than or equal to 3 μm and less than or equal to 8.5 μm (i.e., 3 μm≤X50≤8.5 μm). In an embodiment, the pharmaceutical composition has a particle size distribution by volume such that 50% of the cabotegravir particles have a particle diameter less than or equal to 8 μm (i.e., X50 is 8 μm). In an embodiment, cabotegravir particles of the pharmaceutical composition have an X50 value greater than or equal to 3.5 μm and less than or equal to 8 μm (i.e., 3.5 μm≤X50≤8 μm). In an embodiment, cabotegravir particles of the pharmaceutical composition have an X50 value greater than or equal to 3 μm and less than or equal to 6 μm (i.e., 3 μm≤X50≤6 μm).

In an embodiment, the pharmaceutical composition has a particle size distribution by volume such that 50% of the cabotegravir particles (X50) have a particle diameter smaller than or equal to 3.0 μm, 3.1 μm, 3.2 μm, 3.3 μm, 3.4 μm, 3.5 μm, 3.6 μm, 3.7 μm, 3.8 μm, 3.9 μm, 4.0 μm, 4.1 μm, 4.2 μm, 4.3 μm, 4.4 μm, 4.5 μm, 4.6 μm, 4.7 μm, 4.8 μm, 4.9 μm, 5.0 μm, 5.1 μm, 5.2 μm, 5.3 μm, 5.4 μm, 5.5 μm, 5.6 μm, 5.7 μm, 5.8 μm, 5.9 μm, 6.0 μm, 6.1 μm, 6.2 μm, 6.3 μm, 6.4 μm, 6.5 μm, 6.6 μm, 6.7 μm, 6.8 μm, 6.9 μm, 7.0 μm, 7.1 μm, 7.2 μm, 7.3 μm, 7.4 μm, 7.5 μm, 7.6 μm, 7.7 μm, 7.8 μm, 7.9 μm, or 8.0 μm. In another embodiment, the pharmaceutical composition has a particle size distribution by volume such that 50% of the cabotegravir particles (X50) have a particle diameter smaller than or equal to 4 μm (i.e., X50 is 4 μm). In another embodiment, the pharmaceutical composition has a particle size distribution by volume such that 50% of the cabotegravir particles (X50) have a particle diameter smaller than or equal to 6 μm (i.e., X50 is 6 μm).

In an embodiment, the pharmaceutical composition has a particle size distribution by volume such that 10% of the cabotegravir particles have a particle diameter smaller than or equal to 4 μm (i.e., X10 is 4 μm). In an embodiment, cabotegravir particles of the pharmaceutical composition have an X10 value greater than or equal to 0.5 μm and less than or equal to 4 μm (i.e., 0.5 μm≤X10≤4 μm). In an embodiment, the pharmaceutical composition has a particle size distribution by volume such that 10% of the cabotegravir particles have a particle diameter less than or equal to 3.5 μm. In an embodiment, cabotegravir particles of the pharmaceutical composition have an X10 value greater than or equal to 1 μm and less than or equal to 3.5 μm (i.e., 1 μm≤X10≤3.5 μm). In an embodiment, the pharmaceutical composition has a particle size distribution by volume such that 10% of the cabotegravir particles have a particle diameter less than or equal to 3 μm. In an embodiment, cabotegravir particles of the pharmaceutical composition have an X10 value greater than or equal to 1.5 μm and less than or equal to 3 μm (i.e., 1.5 μm≤X10≤3 μm).

In an embodiment, the pharmaceutical composition has a particle size distribution by volume such that 10% of the cabotegravir particles (X10) have a particle diameter smaller than or equal to 1.0 μm, 1.1 μm, 1.2 μm, 1.3 μm, 1.4 μm, 1.5 μm, 1.6 μm, 1.7 μm, 1.8 μm, 1.9 μm, 2.0 μm, 2.1 μm, 2.2 μm, 2.3 μm, 2.4 μm, 2.5 μm, 2.6 μm, 2.7 μm, 2.8 μm, 2.9 μm, 3.0 μm, 3.1 μm, 3.2 μm, 3.3 μm, 3.4 μm, 3.5 μm, 3.6 μm, 3.7 μm, 3.8 μm, 3.9 μm, or 4.0 μm. In another embodiment, the pharmaceutical composition has a particle size distribution by volume such that 10% of the cabotegravir particles (X10) have a particle diameter smaller than or equal to 1.7 μm (i.e., X10 is 1.7 μm). In another embodiment, the pharmaceutical composition has a particle size distribution by volume such that 10% of the cabotegravir particles (X10) have a particle diameter smaller than or equal to 2.2 μm (i.e., X10 is 2.2 μm). In another embodiment, the pharmaceutical composition has a particle size distribution by volume such that 10% of the cabotegravir particles (X10) have a particle diameter smaller than or equal to 2.6 μm (i.e., X10 is 2.6 μm).

In an embodiment, all X90, X50, and X10 values described herein are by volume and determined by laser diffraction method. The laser diffraction method is sensitive to the volume of a particle and provides a volume-average particle size, which is equivalent to the weight-average particle size if the density is constant. It will be apparent to those skilled in the art that the results of the particle size distribution determination by one technique can be correlated with that from another technique, for example on an empirical basis by routine experimentation. Alternatively, particle size distribution can be determined by microscopy, in particular electron microscopy or scanning electron microscopy.

### Lyophilized Form

For aqueous pharmaceutical suspensions, one pathway of long-term failure is irreversible product settling that creates an inability for these systems to be easily resuspended over the expected product shelf-life. In these cases of physical instability, drug content may not be uniform in the vial at time of administration, which may lead to inconsistent product dosing into patients. The physical stability of suspensions can be greatly influenced by the size of the particle being suspended. Particles size-reduced to under a few hundred nanometers may be stabilized by a variety of factors such as Brownian motion, steric stabilization by excipients, electric stabilization, flocculation, etc., which can all minimize effects of Van der Waals forces between particles that may result in irreversible product settling. However, particles in the range of a few microns may experience greater sedimentation rate and force due to their relatively larger size, which can overpower stabilizing forces separating particles. If these hydrophobic particles directly interact with each other in an aqueous environment, particle agglomeration is likely to occur-leading to irreversible settling. Thus, the inventors' discovery of a lyophilized formulation of micro-particles (as opposed to nano-particles) of cabotegravir is both surprising and unexpected, as reversible settling was observed to occur for micro-particles. Indeed, settling of micro-particle suspension of cabotegravir previously believed to be irreversible was surprisingly shown to be reversible following lyophilization. Due to this reversibility, lyophilized micro-particle suspensions (or microsuspensions) of cabotegravir have unexpectedly extended shelf-life (e.g., at least two years when stored at up to 30° C.).

Lyophilization comprises at least a freezing step and a sublimation step. Lyophilization may be used in the manufacturing of pharmaceutical products and intermediates thereof. During freezing, the material is cooled to a temperature wherein the solid, liquid, and gas phases of the material may exist. Active pharmaceutical product ingredients (APIs) may be lyophilized to achieve chemical and physical stability allowing room temperature storage. This is different from a conventional method that evaporates water using heat. Advantages of lyophilization may include, but are not limited to, enhanced stability of a dry powder, the removal of water without excessive heating of the product, and enhanced product stability in a dry state.

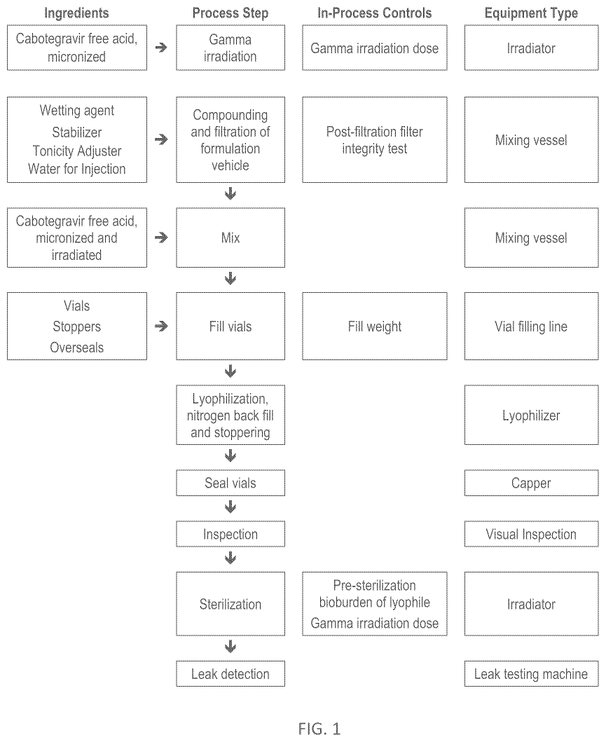

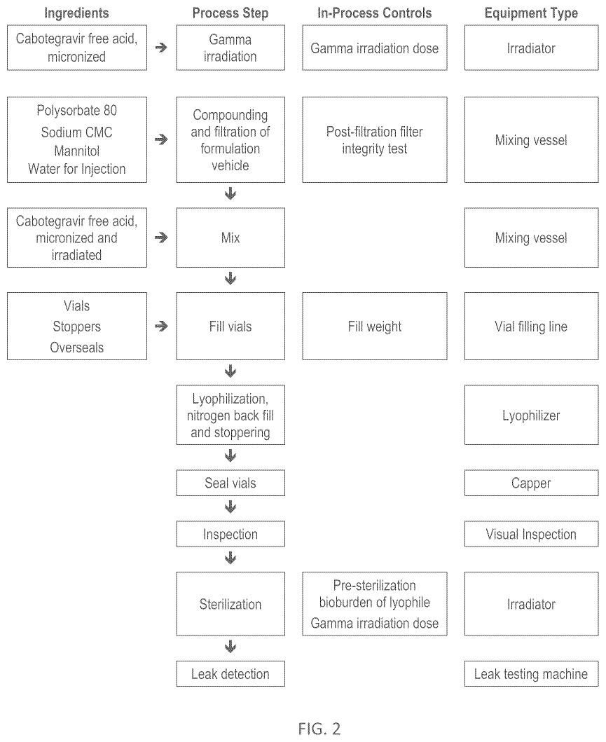

In an exemplary method of preparing a lyophilized formulation of the invention, micronized cabotegravir free acid is packaged in antistatic linear low-density polyethylene bags. The packaged cabotegravir is enclosed in a corrugated plastic box and gamma irradiated as a bioburden reduction step and referred to as gamma irradiated cabotegravir. Gamma irradiated cabotegravir is dispersed in a filtered aqueous vehicle comprising a stabilizer (e.g., sodium CMC), a tonicity agent (e.g., mannitol), and a wetting agent (e.g., PS80). The resulting suspension is filled into washed, sterilized/depyrogenated 10 mL Type I clear glass vials. Container materials are then processed: vials are depyrogenated by dry heat, and stoppers and overseals are sterilized by steam. Product vials are lyophilized, backflushed with nitrogen, sealed with halobutyl rubber stoppers and secured by an aluminum overseal. The sealed vials are terminally sterilized by gamma irradiation. See FIGS. 1 & 2.

As evidenced in the examples, which illustrate certain representative embodiments of the invention, the inventors have developed lyophilized pharmaceutical compositions and methods to obtain said compositions that allow for larger-size particles of cabotegravir. Data provided herein indicate that such lyophilized pharmaceutical compositions reduce injection site reactions and contribute to improved pharmacokinetic properties, thus addressing one or more above-mentioned problems in the art.

In an embodiment, the lyophilized pharmaceutical composition is a suspension. The lyophilized pharmaceutical composition advantageously suspends when reconstituted in an aqueous or non-aqueous solution, that is, all or substantially all, such as at least 90 percent, at least 95 percent, at least 96 percent, at least 97 percent, at least 98 percent, at least 99 percent, at least 99.5 percent or 100 percent of the lyophilized pharmaceutical composition is suspended when reconstituted.

Reconstitution can be assessed visually with the naked eye. The lyophilized matter is deemed reconstituted when a homogeneous suspension is observed. In particular, a suspension with a cloudy appearance is considered suitably reconstituted.

It will be apparent to those skilled in the art that pharmaceutical compositions described herein may be reconstituted in an aqueous or non-aqueous solution to a desired concentration. For example, the pharmaceutical composition described below in Examples 1-3 may be reconstituted in 1.7 mL water to achieve a cabotegravir concentration of 400 mg/mL. Similarly, the same pharmaceutical composition described below in Examples 1-3 may be reconstituted in 1.1 mL water to achieve a cabotegravir concentration of 533 mg/mL.

In an embodiment, the present disclosure provides a lyophilized pharmaceutical composition comprising cabotegravir, wherein cabotegravir is present in the form of particles having a mass median diameter (X50) of between (and including) 2.5 μm and 10 μm; a wetting agent; a stabilizer; and a tonicity adjuster; wherein the formulation, when reconstituted in an aqueous solution, has a reconstitution time of 15 minutes or less, 10 minutes or less, or 5 minutes or less. In another embodiment, the present disclosure provides a lyophilized pharmaceutical composition comprising cabotegravir, wherein cabotegravir is present in the form of particles having a mass median diameter (X50) of between (and including) 2.5 μm and 10 μm; PS80; sodium CMC; and mannitol; wherein the formulation, when reconstituted in an aqueous or non-aqueous solution, has a reconstitution time of 15 minutes or less, 10 minutes or less, or 5 minutes or less.

### Exemplary Embodiments

In an embodiment, the pharmaceutical composition comprises cabotegravir, mannitol, PS80, and sodium CMC; wherein cabotegravir is present in the form of particles having a mass median diameter (X50) of between (and including) 2.5 μm and 10 μm.

In an embodiment, the pharmaceutical composition comprises:

| Ingredient | Mass (mg/vial) |
|---|---|
| Cabotegravir (X10 = 1.7-2.6 μm; X50 = 4-6 | 800 |
| μm; X90 = 9-17 μm) | |
| Mannitol | 51.8-70.0 |
| Polysorbate 80 | 5.9-8.0 |
| Sodium Carboxymethylcellulose | 7.4-10.0 |

While the above embodiment describes vials containing 800 mg cabotegravir, various vial sizes may accommodate larger or smaller amounts of cabotegravir, with proportional changes in amounts of the stabilizer (e.g., sodium carboxymethylcellulose), the tonicity adjuster (e.g., mannitol), and the wetting agent (e.g., polysorbate 80). Reconstitution fill volumes can be adjusted to achieve the desired concentration of cabotegravir. In an embodiment, the pharmaceutical composition is prepared in a vial size of 2, 3, 4, 5, 6, 7, 8, 9, 10, 11, 12, 13, 14, 15, 16, 17, 18, 19, 20, 21, 22, 23, 24, 25, 26, 27, 28, 29, or 30 mL. In an embodiment, the pharmaceutical composition is prepared in a vial size of 25 mL. It is believed that a 25-mL vial improves lyophilization of the pharmaceutical composition, reconstitution time, and extraction from the vial.

In an embodiment, the pharmaceutical composition comprises 800 mg cabotegravir (X50=6 μm); 51.8 mg mannitol; 5.9 mg PS80; and 7.4 mg sodium CMC. In an embodiment, the pharmaceutical composition comprises 800 mg cabotegravir (X10=2.2 μm); 51.8 mg mannitol; 5.9 mg PS80; and 7.4 mg sodium CMC. In an embodiment, the pharmaceutical composition comprises 800 mg cabotegravir (X90=17 μm); 51.8 mg mannitol; 5.9 mg PS80; and 7.4 mg sodium CMC. In an embodiment, the pharmaceutical composition comprises 800 mg cabotegravir (X10=2.2 μm; X50=6 μm; X90=17 μm); 51.8 mg mannitol; 5.9 mg PS80; and 7.4 mg sodium CMC.

In an embodiment, the pharmaceutical composition comprises 800 mg cabotegravir (X50=6 μm); 70.0 mg mannitol; 8.0 mg PS80; and 10.0 mg sodium CMC. In an embodiment, the pharmaceutical composition comprises 800 mg (X10=2.6 μm); 70.0 mg mannitol; 8.0 mg PS80; and 10.0 mg sodium CMC. In an embodiment, the pharmaceutical composition comprises 800 mg cabotegravir (X90=14 μm); 70.0 mg mannitol; 8.0 mg PS80; and 10.0 mg sodium CMC. In an embodiment, the pharmaceutical composition comprises 800 mg cabotegravir (X10=2.6 μm; X50=6 μm; X90=14 μm); 70.0 mg mannitol; 8.0 mg PS80; and 10.0 mg sodium CMC.

In an embodiment, the pharmaceutical composition comprises 800 mg cabotegravir (X50=4 μm); 70.0 mg mannitol; 8.0 mg PS80; and 10.0 mg sodium CMC. In an embodiment, the pharmaceutical composition comprises 800 mg cabotegravir (X10=1.7 μm); 70.0 mg mannitol; 8.0 mg PS80; and 10.0 mg sodium CMC. In an embodiment, the pharmaceutical composition comprises 800 mg cabotegravir (X90=9 μm); 70.0 mg mannitol; 8.0 mg PS80; and 10.0 mg sodium CMC. In an embodiment, the pharmaceutical composition comprises 800 mg cabotegravir (X10=1.7 μm; X50=4 μm; X90=9 μm); 70.0 mg mannitol; 8.0 mg PS80; and 10.0 mg sodium CMC.

In another embodiment, the pharmaceutical composition comprises cabotegravir, wherein cabotegravir is present in the form of particles having an X50 value greater than or equal to 3.5 μm and less than or equal to 8 μm; PS80; sodium CMC; and mannitol; wherein a weight ratio of cabotegravir:PS80:sodium CMC:mannitol is about 100:1:1.25:8.75.

In another embodiment, the pharmaceutical composition comprises cabotegravir, wherein cabotegravir is present in the form of particles having an X value greater than or equal 3.5 μm and less than or equal to 8 μm; PS80; sodium CMC; and mannitol; wherein a weight ratio of cabotegravir:PS80:sodium CMC:mannitol is about 400:3:3.7:25.9.

In another embodiment, the pharmaceutical composition, after lyophilization and reconstitution, is as described in Tables 2a-1 and 2a-2:

TABLE 2a-1

| Ingredient | Concentration (mg/mL) |
|---|---|
| Cabotegravir | 340-500 (X10 = 1.4-3.3 μm; X50 = 3.4-7.5 μm; |
| | X90 = 7.6-21.3 μm) |
| Polysorbate 80 | 2.5-5.0 |
| Sodium CMC | 3.1-6.3 |
| Mannitol | 22-44 |

TABLE 2a-2

| Ingredient | Concentration (mg/ml) |
|---|---|
| Cabotegravir | 340-550 (X10 = 1.4-3.3 μm; X50 = 3.4-7.5 μm; |
| | X90 = 7.6-21.3 μm) |
| Polysorbate 80 | 2.5-5.5 |
| Sodium CMC | 3.1-6.9 |
| Mannitol | 22-50 |

In another embodiment, the pharmaceutical composition, after lyophilization and reconstitution, is as described in Tables 2b-1 and 2b-2:

TABLE 2b-1

| Ingredient | Concentration (mg/mL) |
|---|---|
| Cabotegravir | 450-667 (X10 = 1.4-3.3 μm; X50 = 3.4-7.5 μm; |
| | X90 = 7.6-21.3 μm) |
| Polysorbate 80 | 2.5-5.0 |
| Sodium CMC | 3.1-6.3 |
| Mannitol | 22-44 |

TABLE 2b-2

| Ingredient | Concentration (mg/mL) |
|---|---|
| Cabotegravir | 450-667 (X10 = 1.4-3.3 μm; X50 = 3.4-7.5 μm; |
| | X90 = 7.6-21.3 μm) |
| Polysorbate 80 | 2.5-5.5 |
| Sodium CMC | 3.1-6.9 |
| Mannitol | 22-50 |

In another embodiment, the pharmaceutical composition, after lyophilization and reconstitution with 1.7 or 1.1 mL water, is as described in Table 2c:

TABLE 2c

| | Reconstituted with 1.7 mL | Reconstituted with 1.1 mL |
|---|---|---|
| | water | water |
| Ingredient | Concentration (mg/mL) | |
| Cabotegravir | 400 (X10 = 2.1 μm; | 533 (X10 = 2.2 μm; |
| | X50 = 6 μm; | X50 = 6 μm; |
| | X90 = 17 μm) | X90 = 17 μm) |
| Polysorbate 80 | 3.0 | 4.0 |
| Sodium CMC | 3.7 | 4.9 |
| Mannitol | 25.9 | 34.5 |

In another embodiment, the pharmaceutical composition, after lyophilization and reconstitution with 1.7 or 1.1 mL water, is as described in Table 2d:

TABLE 2d

| | Reconstituted with 1.7 mL | Reconstituted with 1.1 mL |
|---|---|---|
| | water | water |
| Ingredient | Concentration (mg/mL) | |
| Cabotegravir | 400 (X10 = 2.6 μm; | 533 (X10 = 2.6 μm; |
| | X50 = 6 μm; | X50 = 6 μm; |
| | X90 = 14 μm) | X90 = 14 μm) |
| Polysorbate 80 | 4.0 | 5.3 |
| Sodium CMC | 5.0 | 6.7 |
| Mannitol | 35.0 | 46.6 |

In another embodiment, the pharmaceutical composition, after lyophilization and reconstitution with 1.7 or 1.1 mL water, is as described in Table 2e:

TABLE 2e

| | Reconstituted with 1.7 mL | Reconstituted with 1.1 mL |
|---|---|---|
| | water | water |
| Ingredient | Concentration (mg/mL) | |
| Cabotegravir | 400 (X10 = 1.7 μm; | 533 (X10 = 1.7 μm; |
| | X50 = 4 μm; | X50 = 4 μm; |
| | X90 = 9 μm) | X90 = 9 μm) |
| Polysorbate | 4.0 | 5.3 |
| 80 | | |
| Sodium CMC | 5.0 | 6.7 |
| Mannitol | 35.0 | 46.6 |

pH

In an embodiment the pharmaceutical composition of the invention has a pH of about 4 or greater. A pH of about 4 or more reduces pain to the patient if the composition is administered via injection. In an embodiment, the pharmaceutical composition of the invention has a pH of about 6.5. In an alternative embodiment, the pharmaceutical composition has a pH in the range of about 4 to about 8. In an alternative embodiment, the pharmaceutical composition has a pH in the range of about 5 to about 7. In an alternative embodiment, the pharmaceutical composition has a pH in the range of about 6 to about 7.

### Optional Co-Administrations

In an embodiment a pharmaceutical composition described herein is administered in combination with a broadly neutralising antibody. In some embodiments, the broadly neutralizing antibody is selected from the group consisting of VRC01, VRC01-LS, N6, N6LS, VRC07 and VRC07-523. An example of a disclosure of VRC01 is set forth in U.S. Pat. No. 8,637,036. An example of a disclosure of VRC01-LS is set forth in WO 2012/106578. Examples of disclosures of N6 and N6LS are set forth in WO 2016/196975. Examples of disclosures of VRC07 and VRC07-523 are set forth in U.S. Pat. No. 8,637,036. US Patent Publication No. 2014/0322163 A1, WO 2016/196975 and WO 2017/79479.

In an embodiment, the broadly neutralising antibody is an isolated N6 monoclonal antibody or an antigen binding fragment thereof, comprising a heavy chain complementarity determining region (CDRH) having a CDRH1 amino acid sequence of SEQ ID NO: 1, a CDRH2 amino acid sequence of SEQ ID NO: 2, and a CDRH3 amino acid sequence of SEQ ID NO: 3; and a light chain complementarity determining region (CDRL) having a CDRL1 amino acid of SEQ ID NO: 4, a CDRL2 amino acid sequence of SEQ ID NO: 5, and a CDRH3 amino acid sequence of SEQ ID NO: 6.

In an embodiment, the broadly neutralising antibody is an isolated N6LS monoclonal antibody or an antigen binding fragment, comprising a heavy chain complementarity determining region (CDRH) having a CDRH1 amino acid sequence of SEQ ID NO: 1, a CDRH2 amino acid sequence of SEQ ID NO: 2, and a CDRH3 amino acid sequence of SEQ ID NO: 3: a light chain complementarity determining region (CDRL) having a CDRL1 amino acid of SEQ ID NO: 4, a CDRL2 amino acid sequence of SEQ ID NO: 5, and a CDRH3 amino acid sequence of SEQ ID NO: 6; and a recombinant constant domain comprising M428L and N434S mutations. In some embodiments, the antigen binding fragment is a Fv, Fab, F(ab′)₂, scFv or a scFV₂ fragment.

In some embodiments, the broadly neutralising antibody includes an amino acid substitution that increases binding to the FcRn. Several such substitutions are known to the person of ordinary skill in the art, such as substitutions at IgG constant regions T250Q and M428L (see, e.g., Hinton et al., J Immunol, 176:346-356, 2006); M428L and N434S (the “LS” mutation, see, e.g., Zalevsky, et al., Nature Biotechnology, 28:157-159, 2010); N434A (see, e.g., Petkova et al., Int. Immunol, 18:1759-1769, 2006); T307A, E380A, and N434A (see, e.g., Petkova et al., Int. Immunol, 18:1759-1769, 2006); and M252Y, S254T, and T256E (see, e.g., Dall'Acqua et al., J. Biol. Chem., 281:23514-23524, 2006). The disclosed antibodies and antigen binding fragments can be linked to a Fc polypeptide including any of the substitutions listed above, for example, the Fc polypeptide can include the M428L and N434S substitutions. In some embodiments, the antibody comprises a recombinant constant domain comprising a modification that increases binding to a neonatal Fc receptor relative to an unmodified constant domain, wherein the recombinant domain is an IgG1 constant domain comprising M428L and N434S mutations.

In an embodiment the neutralising antibody is N6-LS. N6-LS is a broadly neutralising antibody, which comprises (a) a heavy chain variable region (VH) comprising a heavy chain complementarity determining region (HCDR)1, a HCDR2, and a HCDR3 of the VH set forth as SEQ ID NO: 1; (b) a light chain variable region (VL) comprising a light chain complementarity determining region (LCDR) 1, a LCDR2, and a LCDR3 of the VL set forth as SEQ ID NO: 2 or a VL comprising an amino acid sequence at least 90 percent identical to one of SEQ ID NO: 2; or (c) a combination of (a) and (b); further comprising an IgGI constant domain comprising M428L and N434S mutations; and wherein the antibody or antigen binding fragment specifically binds to HIV-1 gpl20 and neutralizes HIV-1 infection.

In an embodiment, a pharmaceutical composition described herein is administered in combination with a capsid inhibitor, a maturation inhibitor, a nucleoside reverse transcriptase translocation inhibitor (NRTTI), or a non-nucleoside reverse transcriptase inhibitor (NNRTI) (optionally, rilpivirine).

The pharmaceutical composition may be administered in combination with a capsid inhibitor. In an embodiment the capsid inhibitor is a compound of Formula (III), or a pharmaceutically acceptable salt thereof:

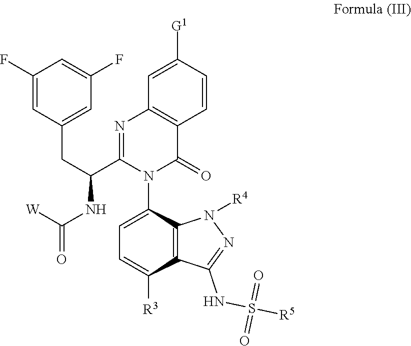

wherein:
G¹ is phenyl substituted once with —N(CH₃)S(O₂)CH₃, —S(O₂)C(CH₃)₃, —CHF₂, —CF₃, —OCHF₂, —OCF₃, or —C(CH₃)₂OH, with the proviso that when G¹ is —CHF₂ or —CF₃, G¹ is not in the para position or G¹ is one of the following:

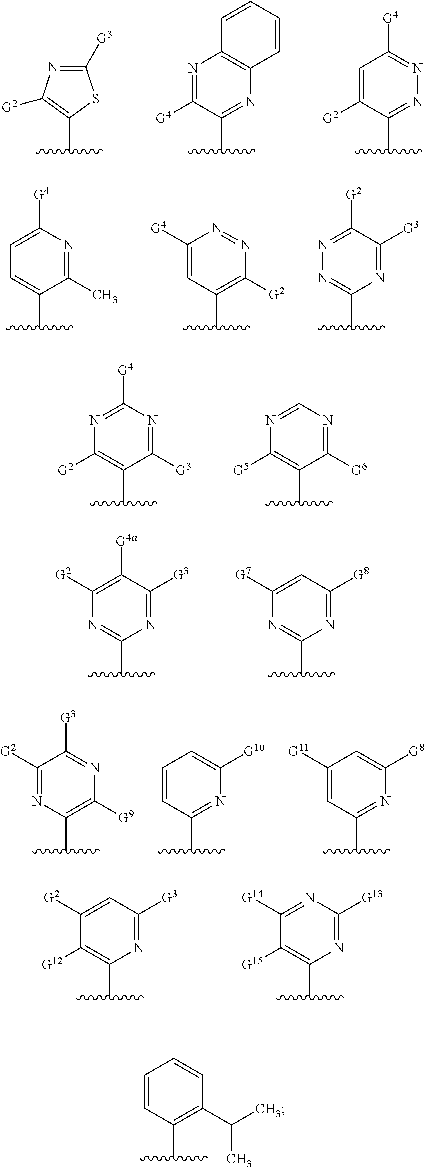

G² and G³ are independently selected from is H or —CH₃;
G⁴ is H, —CH₃, or —OCH₃;
G^{4a} is —CH₃, or —OCH₃;
G⁵ is —CH₃, or CH₂CH₃;
G⁶ is H, —CH₃, or CH₂CH₃;
G⁷ is ethyl, isopropyl, tert-butyl, —CHF₂, or —CF₃;
G⁸ is H, methyl, ethyl, —CHF₂, —CF₃, —OCH₃, or —OCH₂CH₃;
G⁹ is ethyl, isopropyl, cyclopropyl, —CH₂OH, —OCH₃;
G¹⁰ is ethyl, isopropyl, cyclopropyl, tert-butyl, —CHF₂, or —CF₃;
G¹¹ is methyl, —OCH₃, —CHF₂, —CF₃, —S(O₂)CH₃;
G¹² is F, —CH₃, —CHF₂, —CF₃, —OCH₃, —S(O₂)CH₃;
G¹³ is C₁-C₄alkyl, C₁-C₆cycloalkyl, —CH₂O(C₁-C₃alkyl);
G¹⁴ is H, C₁-C₄alkyl, —CHF₂, —CF₃, —O(C₁-C₃alkyl);
G¹⁵ is H, F, —CH₃, or OCH₃;
R³ is H, F, Cl, —CH₃, or —OCH₃;
R⁴ is H or C₁-C₃alkyl wherein C₁-C₃alkyl is optionally substituted with 1-3 fluorines;
R⁵ is C₁-C₆ alkyl or C₃-C₆ cycloalkyl;
W is selected from:

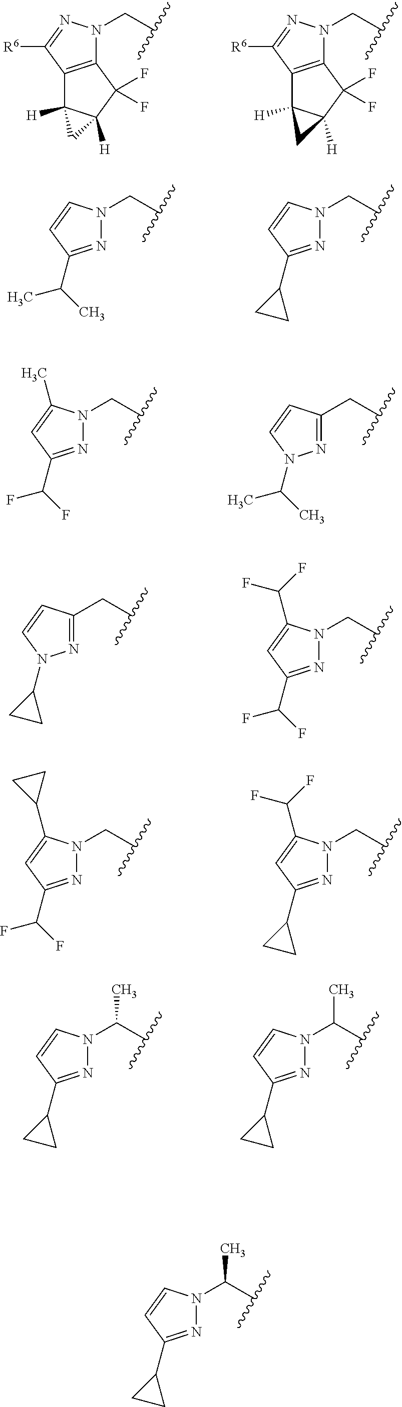

where R⁶ is methyl optionally substituted with 1 to 3 fluorines.

Compounds of Formula (III) are described in WO2020/084492 which is incorporated by reference. In an embodiment the capsid inhibitor is Compound 1 or a pharmaceutically acceptable salt thereof. Compound 1 is described in WO 2020/084492 as Example 59 which example is incorporated herein by reference.

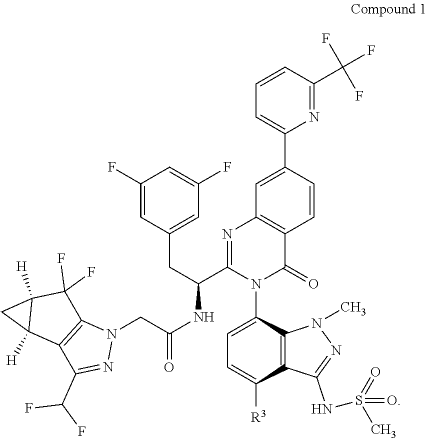

In an embodiment the capsid inhibitor is Compound 2 pharmaceutically acceptable sat thereof. Compound 2 is described in patent application number PCT/IB2020/055653 as Example 1, which example is incorporated herein by reference.

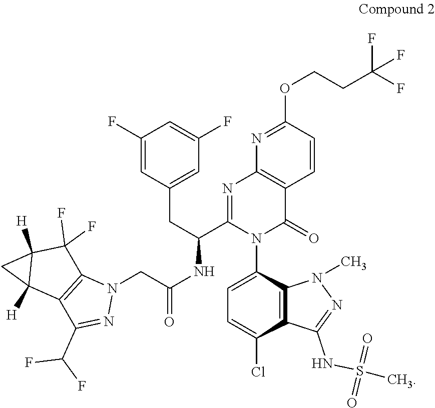

In an alternative embodiment, the capsid inhibitor is lenacapavir.

The pharmaceutical composition may be administered in combination with a maturation inhibitor. In an embodiment the maturation inhibitor is a compound of Formula (IV) or a pharmaceutically acceptable salt thereof:

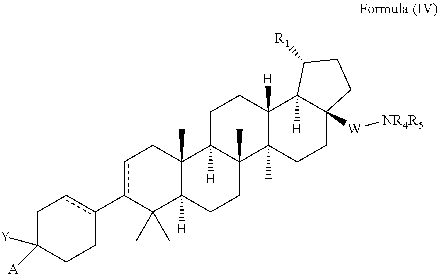

wherein R₁ is isopropenyl or isopropyl;
A is —C₁₋₆ alkyl-OR₀;
wherein R₀ is heteroaryl-Q₀,
Q₀ is selected from the group of —H, —CN, —C₁₋₆ alkyl, —COOH, -Ph, —OC₁₋₆ alkyl, -halo, —CF₃,
Y is selected from the group of —COOR₂, —C(O)NR₂SO₂R₃, —C(O)NHSO₂NR₂R₂, —SO₂NR₂C(O)R₂, tetrazole, and —CONHOH,
wherein n=1-6;
R₂ is —H, —C₁₋₆ alkyl, -alkylsubstituted C₁₋₆ alkyl or -arylsubstituted C₁₋₆ alkyl;
W is absent, or is —CH₂— or —CO—;
R₃ is —H, —C₁₋₆ alkyl or -alkylsubstituted C₁₋₆ alkyl;
R₄ is selected from the group of —H, —C₁₋₆ alkyl, —C₁₋₆ alkyl-C₃₋₆ cycloalkyl, —C₁₋₆ substituted —C₁₋₆ alkyl, —C₁₋₆alkyl-Q₁, —C₁₋₆ alkyl-C₃₋₆ cycloalkyl-Q₁, aryl, heteroaryl, substituted heteroaryl, —COR₆, —SO₂R₇, —SO₂NR₂R₂, and

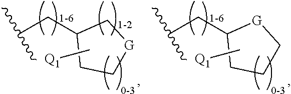

wherein G is selected from the group of —O—, —SO₂— and —NR₁₂—;
wherein Q₁ is selected from the group of —C₁₋₆ alkyl, —C₁₋₆ fluoroalkyl, heteroaryl, substituted heteroaryl, halogen, —CF₃, —OR₂, —COOR₂, —NR₈R₉, —CONR₈R₉ and —SO₂R₇;
R₅ is selected from the group of —H, —C₁₋₆ alkyl, —C₃₋₆ cycloalkyl, —C₁₋₆ alkylsubstituted alkyl, —C₁₋₆ alkyl-NR₈R₉, —COR₃, —SO₂R₇ and —SO₂NR₂R₂;
with the proviso that R₄ or R₅ is not —COR₆ when W is —CO—;
with the further proviso that only one of R₄ or R₅ is selected from the group of —COR₆, —COCOR₆, —SO₂R₇ and —SO₂NR₂R₂;
R₆ is selected from the group of —H, —C₁₋₆ alkyl, —C₁₋₆ alkyl-substitutedalkyl, —C₃₋₆ cycloalkyl, —C₃₋₆ substitutedcycloalkyl-Q₂, —C₁₋₆ alkyl-Q₂, —C₁₋₆ alkyl-substitutedalkyl-Q₂, —C₃₋₆ cycloalkyl-Q₂, aryl-Q₂, —NR₁₃R₁₄, and —OR₁₅;
wherein Q₂ is selected from the group of aryl, heteroaryl, substituted heteroaryl, —OR₂, —COOR₂, —NR₈R₉, SO₂R₇, —CONHSO₂R₃, and —CONHSO₂NR₂R₂;
R₇ is selected from the group of —H, —C₁₋₆ alkyl, —C₁₋₆ substituted alkyl, —C₃₋₆ cycloalkyl, —CF₃, aryl, and heteroaryl;
R₈ and R₉ are independently selected from the group of —H, —C₁₋₆ alkyl, —C₁₋₆ substituted alkyl, aryl, heteroaryl, substituted aryl, substituted heteroaryl, —C₁₋₆ alkyl-Q₂, and —COOR₃,
or R₈ and R₉ are taken together with the adjacent N to form a cycle selected from the group

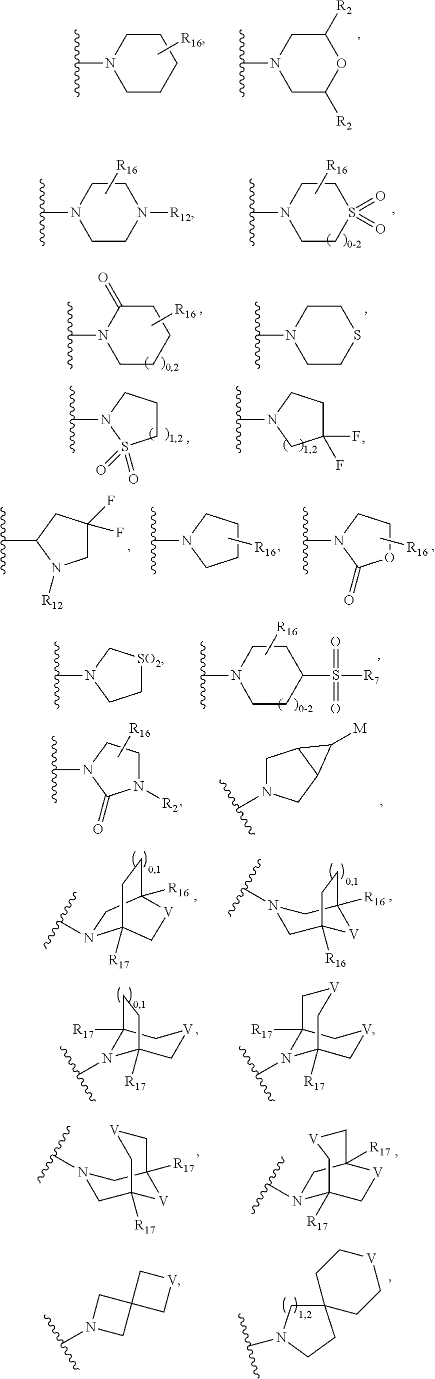

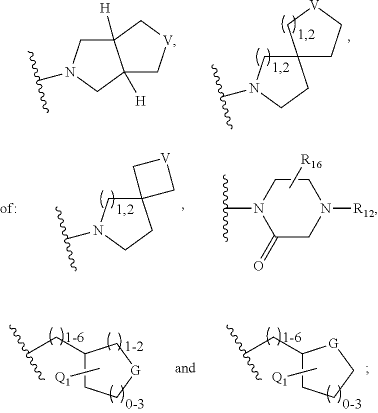

M is selected from the group of —R₁₅, —SO₂R₂, —SO₂NR₂R₂, —OH and —NR₂R₁₂;
V is selected from the group of —CR₁₀R₁₁—, —SO₂—, —O— and —NR₁₂—;
with the proviso that only one of R₈ or R₉ can be —COOR₃;
R₁₀ and R₁₁ are independently selected from the group of —H, —C₁₋₆ alkyl, —C₁₋₆ substituted alkyl and —C₃₋₆ cycloalkyl;
R₁₂ is selected from the group of —H, —C₁₋₆ alkyl, -alkylsubstituted C₁₋₆ alkyl, —CONR₂R₂, —SO₂R₃, and —SO₂NR₂R₂,
R₁₃ and R₁₄ are independently selected from the group of —H, —C₁₋₆ alkyl, —C₃₋₆ cycloalkyl, —C₁₋₆ substituted alkyl, —C₁₋₆ alkyl-Q₃, —C₁₋₆ alkyl-C₃₋₆ cycloalkyl-Q₃, and C₁₋₆ substituted alkyl-Q₃;
Q₃ is selected from the group of heteroaryl, substituted heteroaryl, —NR₂R₁₂, —CONR₂R₂, —COOR₂, —OR₂, and —SO₂R₃;
R₁₅ is selected from the group of —C₁₋₆ alkyl, —C₃₋₆ cycloalkyl, —C₁₋₆ substituted alkyl, —C₁₋₆ alkyl-Q₃, —C₁₋₆ alkyl-C₃₋₆ cycloalkyl-Q₃ and —C₁₋₆ substituted alkyl-Q₃;
R₁₆ is selected from the group of —H, —C₁₋₆ alkyl, —NR₂R₂, and —COOR₂;
with the proviso that when V is —NR₁₂—; R₁₆ is not —NR₂R₂; and
R₁₇ is selected from the group of —H, —C₁₋₆ alkyl, —COOR₃, and aryl.

Compounds of Formula (IV) are described in WO 2017/134596 which is incorporated herein by reference. In an embodiment the maturation inhibitor is Compound 3. Compound 3 is described in WO 2017/134596 as Example 25 which example is incorporated herein by reference.

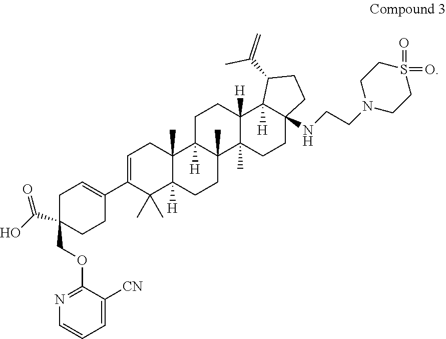

The pharmaceutical composition may be administered in combination with an NRTTI. In an embodiment the NRTTI is a compound of the formula (V):

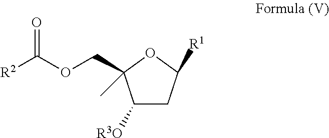

wherein:
R¹ is:

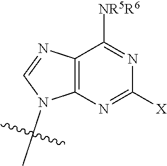

wherein:
X is selected from the group consisting of NH₂, F and Cl;
R⁵ is selected from the group consisting of H and (C₁-C₁₄) alkyl;
R⁶ is selected from the group consisting of H and —(C═O)—(C₁-C₁₄) alkyl;
R² is selected from the group consisting of (C₁-C₂₄) alkyl; (CH₂)ₙ₁—O—(CH₂CH₂O)ₙ₂—(C₁-C₁₄ alkyl) where n1 and n2 are integers independently selected from 1-4; —R⁷—NH—(C═O)—R⁸ wherein R⁷ may be (C₁-C₁₄) alkyl and R⁸ may be independently selected from H and (C₁-C₁₄) alkyl; —R⁹—(C₆-C₁₄) aryl, wherein R⁹ is a bond or (C₁-C₆) alkyl; —R¹⁰—(C₃-C₁₄) cycloalkyl, wherein R¹⁰ is a bond or (C₁-C₆) alkyl; —(C₁-C₂₀) alkylene-(C═O)—O—R¹¹ wherein R¹¹ may be selected from H and (C₁-C₂₀)alkyl;

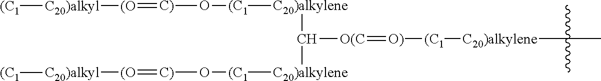

and;
R³ is selected from the group consisting of H, —(C═O)—(C₁-C₂₄) alkyl; —(C═O)—O—(C₁-C₂₄) alkyl; and C₃-C₁₄ cycloalkyl; or
R² and R³ join together to form a C₃ to C₂₈ cyclic structure; and
with the proviso that when R² is (C₁-C₁₄ alkyl) at least one of R³, R⁵ and R⁶ is not H.

Compounds of Formula 3 are disclosed in WO 2020/178767 which is incorporated by reference herein. In an embodiment a pharmaceutical composition of the invention is combined with Compound 4. Compound 4 is described in WO 2020/178767 as Example 18, which example is incorporated by reference herein.

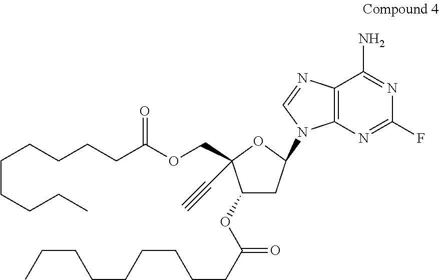 [SMILES: C#C[C@]1(COC(=O)CCCCCCCCC)O[C@@H](n2cnc3c(N)nc(F)nc32)C[C@@H]1OC(=O)CCCCCCCCC]

In an alternative embodiment the NRTTI is Islatravir.

### Methods for Treatment and Prevention of HIV

In a second aspect, the present invention provides methods for (a) treatment of HIV in a human in need thereof comprising administering to said human a therapeutically effective amount of a pharmaceutical composition as defined herein; and (b) prevention of HIV in a human comprising administering to said human an effective amount of a pharmaceutical composition as defined herein.

In one embodiment, the method comprises administering the pharmaceutical composition parenterally. In an embodiment, the pharmaceutical composition is administered intramuscularly. In an embodiment, the pharmaceutical composition is administered subcutaneously.

In an embodiment the method comprises administering around 1 mL to around 8 mL of the pharmaceutical composition to a patient. In an embodiment the method comprises administering around 1 mL of the pharmaceutical composition to a patient. In another embodiment the method comprises administering around 2 mL of the pharmaceutical composition to a patient. In an embodiment the method comprises administering around 3 mL of the pharmaceutical composition to a patient. In an embodiment the method comprises administering around 4 mL of the pharmaceutical composition to a patient. In an embodiment the method comprises administering around 5 mL of the pharmaceutical composition to a patient. In an embodiment the method comprises administering around 6 mL of the pharmaceutical composition to a patient. In an embodiment the method comprises administering around 7 mL of the pharmaceutical composition to a patient. In an embodiment the method comprises administering around 8 mL of the pharmaceutical composition to a patient.

In an embodiment, the pharmaceutical composition is administered in more than one injection. In an embodiment, the pharmaceutical composition is administered in two or more injections, which may be simultaneously or consecutively administered. For example, two separate injections of 3 mL (for a total of 6 mL) of the pharmaceutical composition may be consecutively administered to the patient. In an embodiment, the pharmaceutical composition is administered in two injections. In an embodiment, the pharmaceutical composition is administered as a 1 mL injection. In an embodiment, the pharmaceutical composition is administered as a 2 mL injection. In an embodiment, the pharmaceutical composition is administered as a 3 mL injection.

In an embodiment, about 300 mg to about 3200 mg of cabotegravir is administered to the patient in the pharmaceutical composition.

In an embodiment, the pharmaceutical composition has a cabotegravir concentration of about 400 mg/mL and about 800 mg to about 1600 mg of cabotegravir is administered to the patient. In an embodiment, the pharmaceutical composition has a cabotegravir concentration of about 400 mg/mL and 800 mg of cabotegravir is administered to the patient. In an embodiment the pharmaceutical composition has a cabotegravir concentration of about 400 mg/mL and 1200 mg of cabotegravir is administered to the patient.

In an embodiment the pharmaceutical composition has a cabotegravir concentration of about 533 mg/mL and about 1200 to about 3200 mg of cabotegravir is administered to the patient. In an embodiment the pharmaceutical composition has a cabotegravir concentration of about 533 mg/mL and about 1600 mg of cabotegravir is administered to the patient. In an embodiment the pharmaceutical composition comprises about 1600 mg of cabotegravir in a 3 mL injection. In an embodiment the pharmaceutical composition has a cabotegravir concentration of about 533 mg/mL and about 2400 mg of cabotegravir is administered to the patient. In an embodiment the pharmaceutical composition comprises about 2400 mg of cabotegravir in two 2 mL injections. In an embodiment the pharmaceutical composition has a cabotegravir concentration of about 533 mg/mL and about 3200 mg of cabotegravir is administered to the patient. In an embodiment the pharmaceutical composition comprises about 3200 mg of cabotegravir in three 3 mL injections.

In an embodiment the pharmaceutical composition is administered to a patient once every 1, 2, 3, 4, 5, or 6 months. In an embodiment, the pharmaceutical composition is administered to the human once every month. In an alternative embodiment, the pharmaceutical composition is administered once every two months. In an alternative embodiment, the pharmaceutical composition is administered once every three months. In an alternative embodiment, the pharmaceutical composition is administered once every four months. In an alternative embodiment, the pharmaceutical composition is administered once every five months. In an alternative embodiment, the pharmaceutical composition is administered once every six months. In an alternative embodiment, the pharmaceutical composition is administered once every 15 to 24 weeks. In an embodiment, the pharmaceutical composition is administered once every 15 to 20 weeks, or once every 15 to 19 weeks.

In an embodiment the pharmaceutical composition is administered once every 16 to 18 weeks. In an embodiment, the pharmaceutical composition is administered once every 16 weeks, or once every 17 weeks, or once every 18 weeks.

In an embodiment, the pharmaceutical composition may be administered by any suitable means.

In one embodiment, pharmaceutical composition may be administered subcutaneously. In this embodiment, the pharmaceutical composition may be administered subcutaneously by another (e.g., by a healthcare professional) or may be self-administered by a patient. In this embodiment the pharmaceutical composition may be administered subcutaneously via injection. In an embodiment the pharmaceutical composition is administered subcutaneously via injection. In an embodiment of the invention the pharmaceutical composition is administered or self-administered once monthly by subcutaneous injection. In another embodiment, the pharmaceutical composition is administered or self-administered once every two months by subcutaneous injection. In another embodiment, the pharmaceutical composition is administered or self-administered once every three months by subcutaneous injection. In another embodiment, the pharmaceutical composition is administered or self-administered once every four months by subcutaneous injection. In another embodiment, the pharmaceutical composition is administered or self-administered once every five months by subcutaneous injection. In another embodiment, the pharmaceutical composition is administered or self-administered once every six months by subcutaneous injection. In an embodiment, the pharmaceutical composition is administered or self-administered in one injection. In another embodiment, the pharmaceutical composition is administered or self-administered in two or more injections, which may be simultaneously or consecutively administered. In an embodiment, the pharmaceutical composition is administered or self-administered in two injections.

In another embodiment the pharmaceutical composition is administered intramuscularly via injection. In this embodiment, the pharmaceutical composition may be administered intramuscularly by another (e.g., by a healthcare professional) or may be self-administered by a patient. In an embodiment of the invention, the pharmaceutical composition is administered or self-administered intramuscularly via injection once every month. In another embodiment, the pharmaceutical composition is administered or self-administered intramuscularly via injection once every two months. In another embodiment, the pharmaceutical composition is administered or self-administered intramuscularly via injection once every three months. In another embodiment, the pharmaceutical composition is administered or self-administered intramuscularly via injection once every four months. In another embodiment, the pharmaceutical composition is administered or self-administered intramuscularly via injection once every five months. In another embodiment, the pharmaceutical composition is administered or self-administered intramuscularly via injection once every six months. In an embodiment the intramuscular injection is administered by a healthcare professional. In an embodiment, the pharmaceutical composition is intramuscularly administered in one injection during a visit with a healthcare professional. In another embodiment, the pharmaceutical composition is intramuscularly administered in two or more injections, which may be simultaneously or consecutively administered, during one visit with a healthcare professional. In an embodiment, the pharmaceutical composition is intramuscularly administered in two injections, which may be simultaneously or consecutively administered, during one visit with a healthcare professional. In another embodiment, the pharmaceutical composition is intramuscularly self-administered in one injection. In another embodiment, the pharmaceutical composition is intramuscularly self-administered in two or more injections, which may be simultaneously or consecutively self-administered. In an embodiment, the pharmaceutical composition is intramuscularly self-administered in two injections, which may be simultaneously or consecutively self-administered.

In an embodiment the pharmaceutical compositions of the present invention are administered in combination with other pharmaceutical compositions as a component of a multi drug treatment regimen. In an embodiment, the other pharmaceutical compositions are drugs which treat or prevent HIV. Marketed medicines are currently available to treat HIV.

In an embodiment the pharmaceutical compositions of the present invention are administered in combination with N6-LS. N6-LS is described above.

In an embodiment the pharmaceutical compositions of the present invention are administered in combination with a capsid inhibitor, a maturation inhibitor or a nucleoside reverse transcriptase translocation inhibitor (NRTTI), or a non-nucleoside reverse transcriptase inhibitor (NNRTI) (optionally, rilpivirine).

### Use in Treatment or Prevention of HIV

In a third aspect, the present invention provides a pharmaceutical composition defined herein for use in the treatment or prevention of HIV.

In one embodiment the use comprises administering the pharmaceutical composition parenterally. In one embodiment, the pharmaceutical composition is suitable for use as an injectable composition. In an embodiment the use comprises administering the pharmaceutical composition intramuscularly. In another embodiment the use comprises administering the pharmaceutical composition subcutaneously.

In an embodiment the use comprises administering around 1 mL to around 8 mL of the pharmaceutical composition to a patient. In an embodiment the use comprises administering around 1 mL of the pharmaceutical composition to a patient. In another embodiment the use comprises administering around 2 mL of the pharmaceutical composition to a patient. In an embodiment the use comprises administering around 3 mL of the pharmaceutical composition to a patient. In an embodiment the use comprises administering around 4 mL of the pharmaceutical composition to a patient. In an embodiment the use comprises administering around 5 mL of the pharmaceutical composition to a patient. In an embodiment the use comprises administering around 6 mL of the pharmaceutical composition to a patient. In an embodiment the use comprises administering around 7 mL of the pharmaceutical composition to a patient. In an embodiment the use comprises administering around 8 mL of the pharmaceutical composition to a patient.

In an embodiment, the use comprises administering the pharmaceutical composition in more than one injection. In an embodiment, the use comprises administering the pharmaceutical composition in two or more injections, which may be simultaneously or consecutively administered. For example, two separate injections of 3 mL (for a total of 6 mL) of the pharmaceutical composition may be consecutively administered to the patient. In an embodiment, the use comprises administering the pharmaceutical composition in two injections.

In an embodiment, about 300 mg to about 3200 mg of cabotegravir is administered to the patient in the pharmaceutical composition.

In an embodiment, the pharmaceutical composition has a cabotegravir concentration of about 400 mg/mL and about 800 mg to about 1600 mg of cabotegravir is administered to the patient. In an embodiment, the pharmaceutical composition has a cabotegravir concentration of about 400 mg/mL and 800 mg of cabotegravir is administered to the patient. In an embodiment the pharmaceutical composition has a cabotegravir concentration of about 400 mg/mL and 1200 mg of cabotegravir is administered to the patient.

In an embodiment the pharmaceutical composition has a cabotegravir concentration of about 533 mg/mL and about 1200 to about 3200 mg of cabotegravir is administered to the patient. In an embodiment the pharmaceutical composition has a cabotegravir concentration of about 533 mg/mL and about 1600 mg of cabotegravir is administered to the patient. In an embodiment the pharmaceutical composition has a cabotegravir concentration of about 533 mg/mL and about 2400 mg of cabotegravir is administered to the patient. In an embodiment the pharmaceutical composition has a cabotegravir concentration of about 533 mg/mL and about 3200 mg of cabotegravir is administered to the patient.

In an embodiment the use comprises administering the pharmaceutical to a patient once every 1, 2, 3, 4, 5, or 6 months. In an embodiment the use comprises administering the pharmaceutical composition to a patient once every month. In an alternative embodiment, the use comprises administering the pharmaceutical composition once every two months. In an alternative embodiment the use comprises administering the pharmaceutical composition once every three months. In an alternative embodiment the use comprises administering the pharmaceutical composition once every four months. In an alternative embodiment the use comprises administering the pharmaceutical composition once every five months. In an alternative embodiment the use comprises administering the pharmaceutical composition once every six months.

In an embodiment, use comprises administering the pharmaceutical composition by any suitable means.

In one embodiment, the use comprises the patient self-administering the pharmaceutical composition. In this embodiment the use may comprise administering the pharmaceutical composition subcutaneously via injection. In one embodiment of the invention the use comprises self-administering the pharmaceutical composition once monthly by subcutaneous injection. In another embodiment, the use comprises self-administering the pharmaceutical composition once every two months by subcutaneous injection. In another embodiment, the use comprises self-administering the pharmaceutical composition once every three months by subcutaneous injection. In another embodiment, the use comprises self-administering the pharmaceutical composition once every four months by subcutaneous injection. In another embodiment, the use comprises self-administering the pharmaceutical composition once every five months by subcutaneous injection. In another embodiment, the use comprises self-administering the pharmaceutical composition once every six months by subcutaneous injection. In an embodiment, the use comprises self-administering the pharmaceutical composition in one injection. In another embodiment, the use comprises self-administering the pharmaceutical composition in two or more injections, which may be simultaneously or consecutively administered. In an embodiment, the use comprises self-administering the pharmaceutical composition in two injections.

In another embodiment the use comprises administering the pharmaceutical composition intramuscularly via injection. In an embodiment of the invention, the use comprises administering the pharmaceutical composition intramuscularly via injection once every month. In another embodiment, the use comprises administering the pharmaceutical composition intramuscularly via injection once every two months. In another embodiment, the use comprises administering the pharmaceutical composition intramuscularly via injection once every three months. In another embodiment, the use comprises administering the pharmaceutical composition intramuscularly via injection once every four months. In another embodiment, the use comprises administering the pharmaceutical composition intramuscularly via injection once every five months. In another embodiment, the use comprises administering the pharmaceutical composition intramuscularly via injection once every six months. In an embodiment the intramuscular injection is administered by a healthcare professional. In an embodiment, the use comprises intramuscularly administering the pharmaceutical composition in one injection during a visit with a healthcare professional. In another embodiment, the use comprises intramuscularly administering the pharmaceutical composition in two or more injections, which may be simultaneously or consecutively administered, during one visit with a healthcare professional. In an embodiment, the use comprises intramuscularly administering the pharmaceutical composition in two injections during one visit with a healthcare professional.

In an embodiment of the invention use comprises administering the pharmaceutical compositions of the present invention in combination with other pharmaceutical compositions as a component of a multi drug treatment regimen. In an embodiment, the other pharmaceutical compositions are drugs which treat or prevent HIV. Marketed medicines are currently available to treat HIV.

In an embodiment the use comprises administering the pharmaceutical compositions of the present invention in combination with N6-LS. N6-LS is described above.

In an embodiment the use comprises administering the pharmaceutical compositions of the present invention in combination with a capsid inhibitor, a maturation inhibitor or a nucleoside reverse transcriptase translocation inhibitor (NRTTI), or a non-nucleoside reverse transcriptase inhibitor (NNRTI) (optionally, rilpivirine).

### Kit

In a fourth aspect the present invention provides a kit comprising cabotegravir, wherein cabotegravir is present in the form of particles having an X50 value greater than or equal to 2.5 μm and less than or equal to 10 μm; a wetting agent; a stabilizer; and a tonicity adjuster.

In an embodiment, the present invention provides a kit comprising a container that contains a pharmaceutical composition according to the invention as a lyophilized powder. In an embodiment the kit further comprises a container comprising a liquid suitable to produce a reconstituted solution. In an embodiment the suitable liquid is water. In an embodiment, the suitable liquid is water and the container comprising the water is a prefilled syringe. In an embodiment the kit further comprises a needle suitable for dispensing water from the prefilled syringe to the container comprising the lyophilized composition of the invention. In an alternative embodiment the kit comprises a needle suitable for injecting the reconstituted solution to a patient in need thereof. In a still further embodiment, the kit comprises a needle suitable for dispensing water from the prefilled syringe to the container comprising the lyophilized composition of the invention and injecting the reconstituted solution to a patient in need thereof. In an embodiment the kit further comprises a leaflet comprising use instructions.

In an embodiment the kit comprises a syringe or vial comprising a composition of the invention.

In an embodiment the present invention provides a method of preparing a reconstituted solution, the method comprising providing the kit as described here and contacting the lyophilized composition with a suitable liquid to produce a reconstituted solution. In an embodiment the suitable liquid is an aqueous solvent. In an embodiment the suitable liquid is water. In another embodiment the suitable liquid is a non-aqueous solvent.

The present disclosure further provides the following embodiments:
(a) A parenteral pharmaceutical composition comprising a wetting agent, a stabilizer, a tonicity adjuster, and an effective amount of cabotegravir, for the long term treatment of HIV infection, or prevention of HIV infection in an individual at risk of being infected by HIV, wherein the composition is administered intermittently at a time interval of at least one month.
(b) The composition according to (a) wherein the composition is administered once every 2 months.
(c) The composition according to (a) wherein the composition is administered once every 3 months.
(d) The composition according to (a) wherein the composition is administered once every 4 months.
(e) The composition according to (a) wherein the composition is administered once every 5 months.
(f) The composition according to (a) wherein the composition is administered once every 6 months.
(g) The composition according to any one of (a) to (f) wherein the wetting agent is polysorbate 80, the stabilizer is sodium carboxymethylcellulose, and the tonicity adjuster is mannitol.
(h) The composition according to any one of (a) to (g) wherein the effective amount of cabotegravir is selected such that the blood plasma concentration of cabotegravir in a subject is kept during a prolonged period of time at a level between a maximum blood plasma level which is the blood plasma level that causes significant side effects and the minimum blood plasma level that is the lowest blood plasma level that causes cabotegravir to provide effective treatment or prevention of HIV infection.
(i) The composition according to (h) wherein the blood plasma level of a subject is kept at a level equal to or above about 150 ng/ml, in particular equal to or above about 600 ng/ml.
(j) The composition according to any one of (a) to (i), wherein the composition is administered subcutaneously or intramuscularly.

The dose of cabotegravir administered, which is the amount of cabotegravir in the parenteral composition for use in the invention, may be selected such that: the blood plasma concentration of cabotegravir in the human is kept above a trough plasma concentration (Ctau); or the blood plasma concentration of the human is maintained at or above the Ctau of the approved 200 mg/mL cabotegravir regimen i.e. in the dosing regimen for APRETUDE®.

Trough plasma level (Ctau) refers to trough plasma concentration i.e. the concentration reached immediately before the next dose is administered. The Ctau value represents the lowest blood plasma level. The Ctau can be measured in any suitable way.

The present inventors have found that, to maintain an effective dosing regimen for treatment or prevention of HIV, the dose of cabotegravir, which is the amount of cabotegravir in the pharmaceutical composition for use in the invention, should maintain Ctau at a level higher than the 10th percentile of Ctau observed in the Phase 3 studies 201738 (HPTN 083) for people assigned male at birth (1.05 μg/mL) and 201739 (HPTN 084) for people assigned female at birth (1.39 μg/mL). The 10th percentile of Ctau observed in the Phase 3 studies mentioned are the sex-specific PrEP benchmarks which were achieved in at least 90% of participants.

The present inventors have also found that, to maintain an effective dosing regimen for treatment or prevention of HIV, the dose of cabotegravir needs to maintain the median and 10th percentile of Ctau higher than those from the approved 200 mg/mL intramuscular regimen of cabotegravir (APRETUDE®) in both people who were assigned male at birth and people who were assigned female at birth.

The blood plasma levels of cabotegravir in a human may be kept above these Ctau levels because at lower levels the drug may no longer be effective, thereby increasing the risk of transmission of HIV infection, and may be suboptimal for treatment of HIV infected subjects. Plasma levels of cabotegravir may be kept at higher levels to avoid the development of HIV mutations, while maintaining a safety margin.

### Embodiments

A non-exhaustive list of embodiments of the disclosure is provided below:
Embodiment 1: A pharmaceutical composition comprising:
   cabotegravir;
   a wetting agent;
   a stabilizer; and
   a tonicity adjuster;
   wherein cabotegravir is present in the form of particles having a mass median diameter (X50) of between (and including) 2.5 μm and 10 μm.
Embodiment 2: The pharmaceutical composition according to Embodiment 1, wherein the wetting agent is selected from the group consisting of polysorbate 20, polysorbate 80, sorbitan monolaurate, sorbitan monooleate, poloxamer 188, poloxamer 338, and poloxamer 407.
Embodiment 3: The pharmaceutical composition according to Embodiment 1 or Embodiment 2, wherein the wetting agent is polysorbate 80.
Embodiment 4: The pharmaceutical composition according to any one of Embodiments 1-3, wherein the stabilizer is selected from the group consisting of sodium carboxymethylcellulose, polyethylene glycol 3350, polyethylene glycol 4000, povidone K12, and povidone K17.
Embodiment 5: The pharmaceutical composition according to any one of Embodiments 1-4, wherein the stabilizer is sodium carboxymethylcellulose.
Embodiment 6: The pharmaceutical composition according to any one of Embodiments 1-5, wherein the tonicity adjuster is selected from the group consisting of mannitol, sorbitol, lactose, trehalose, raffinose, dextrose, maltose, galactose, sucrose, and polysucrose.
Embodiment 7: The pharmaceutical composition according to any one of Embodiments 1-6, wherein the tonicity adjuster is mannitol.
Embodiment 8: The pharmaceutical composition according to any one of Embodiments 1-7, wherein the cabotegravir particles have an X90 value greater than or equal to 5 μm and less than or equal to 25 μm.
Embodiment 9: The pharmaceutical composition according to any one of Embodiments 1-8, wherein the cabotegravir particles have an X50 value greater than or equal to 3 μm and less than or equal to 8.5 μm.
Embodiment 10: The pharmaceutical composition according to Embodiment 9, wherein the cabotegravir particles have an X50 value greater than or equal to 3 μm and less than or equal to 8.5 μm, and wherein the cabotegravir particles have an X90 value greater than or equal to 6 μm and less than or equal to 20 μm.
Embodiment 11: The pharmaceutical composition according to any one of Embodiments 1-10, wherein the cabotegravir particles have an X50 value greater than or equal to 3.5 μm and less than or equal to 8.0 μm.
Embodiment 12: The pharmaceutical composition according to any one of Embodiments 1-11, wherein the cabotegravir particles have an X50 value greater than or equal to 3.5 μm and less than or equal to 8.0 μm, and wherein the cabotegravir particles have an X90 value greater than or equal to 7.0 μm and less than or equal to 18.0 μm.
Embodiment 13: The pharmaceutical composition according to any one of Embodiments 1-12, wherein cabotegravir is present in an amount of from about 300 mg to about 1800 mg.
Embodiment 14: The pharmaceutical composition according to any one of Embodiments 1-12, wherein cabotegravir is present in an amount of from about 350 mg to about 1650 mg.
Embodiment 15: The pharmaceutical composition according to any one of Embodiments 1-12, wherein cabotegravir is present in an amount of about 800 mg.
Embodiment 16: The pharmaceutical composition according to any one of Embodiments 1-12, wherein cabotegravir is present in an amount of about 1600 mg.
Embodiment 17: The pharmaceutical composition according to any one of Embodiments 1-16, wherein a weight ratio of the wetting agent to cabotegravir is in a range of from about 1:10 to about 1:400.
Embodiment 18: The pharmaceutical composition according to any one of Embodiments 1-17, wherein a weight ratio of the wetting agent to cabotegravir is in a range of from about 1:50 to about 1:200.
Embodiment 19: The pharmaceutical composition according to any one of Embodiments 1-18, wherein a weight ratio of the wetting agent to cabotegravir is in a range of from about 1:100 to about 1:150.
Embodiment 20: The pharmaceutical composition according to any one of Embodiments 1-19, wherein a weight ratio of the stabilizer to cabotegravir is in a range of from about 1:10 to about 1:400.
Embodiment 21: The pharmaceutical composition according to any one of Embodiments 1-20, wherein a weight ratio of the stabilizer to cabotegravir is in a range of from about 1:40 to about 1:200.
Embodiment 22: The pharmaceutical composition according to any one of Embodiments 1-21, wherein a weight ratio of the stabilizer to cabotegravir is in a range of from about 1:70 to about 1:120.
Embodiment 23: The pharmaceutical composition according to any one of Embodiments 1-22, wherein a weight ratio of the tonicity adjuster to cabotegravir is in a range of from about 1:1 to about 1:100.
Embodiment 24: The pharmaceutical composition according to any one of Embodiments 1-23, wherein a weight ratio of the tonicity adjuster to cabotegravir is in a range of from about 1:5 to about 1:50.
Embodiment 25: The pharmaceutical composition according to any one of Embodiments 1-24, wherein a weight ratio of the tonicity adjuster to cabotegravir is in a range of from about 1:8 to about 1:25.
Embodiment 26: The pharmaceutical composition according to any one of Embodiments 1-25, wherein the wetting agent is polysorbate 80, wherein the wherein the stabilizer is sodium carboxymethylcellulose, wherein the tonicity adjuster is mannitol, and wherein a weight ratio of cabotegravir:PS80:sodium CMC:mannitol is about 100:1:1.25:8.75.
Embodiment 27: The pharmaceutical composition according to any one of Embodiments 1-25, wherein the wetting agent is polysorbate 80, wherein the wherein the stabilizer is sodium carboxymethylcellulose, wherein the tonicity adjuster is mannitol, and wherein a weight ratio of cabotegravir:PS80:sodium CMC:mannitol is about 400:3:3.7:25.9.
Embodiment 28: The pharmaceutical composition according to any one of Embodiments 1-27, wherein the pharmaceutical composition is provided as a lyophilized powder.
Embodiment 29: The pharmaceutical composition according to any one of Embodiments 1-28, wherein a concentration of cabotegravir is in a range of from about 100 mg/mL to about 800 mg/mL.
Embodiment 30: The pharmaceutical composition according to Embodiment 29, wherein the concentration of cabotegravir is in a range of from about 200 mg/mL to about 700 mg/mL.
Embodiment 31: The pharmaceutical composition according to Embodiment 29, wherein the concentration of cabotegravir is in a range of from about 300 mg/mL to about 650 mg/mL.
Embodiment 32: The pharmaceutical composition according to Embodiment 29, wherein the concentration of cabotegravir is about 400 mg/mL.
Embodiment 33: The pharmaceutical composition according to Embodiment 29, wherein the concentration of cabotegravir is about 533 mg/mL.
Embodiment 34: The pharmaceutical composition according to any one of Embodiments 1-33, wherein the pharmaceutical composition is
   (a) reconstituted from a lyophilized powder with a suitable liquid, or
   (b) provided as a liquid composition.
Embodiment 35: The pharmaceutical composition according to Embodiment 34, wherein the suitable liquid is an aqueous solvent.
Embodiment 36: The pharmaceutical composition according to Embodiment 34 or Embodiment 35, wherein the suitable liquid is water.
Embodiment 37: The pharmaceutical composition according to Embodiment 34, wherein the suitable liquid is a non-aqueous solvent.
Embodiment 38: The pharmaceutical composition according to any one of Embodiments 1-37, wherein the pharmaceutical composition is formulated as a parenteral pharmaceutical composition.
Embodiment 39: The pharmaceutical composition according to any one of Embodiments 1-38, wherein the pharmaceutical composition is suitable for injection.
Embodiment 40: The pharmaceutical composition according to Embodiment 39, wherein the pharmaceutical composition is suitable for subcutaneous, subdermal, or intramuscular injection.
Embodiment 41: A pharmaceutical composition according to any one of Embodiments 1-40 for use in treatment or prevention of HIV.
Embodiment 42: The pharmaceutical composition for use according to Embodiment 41, wherein the use comprises a step of administering the pharmaceutical composition subcutaneously or intramuscularly to a human.
Embodiment 43: The pharmaceutical composition for use according to Embodiment 42, wherein about 300 mg to about 3200 mg of cabotegravir is administered to the human.
Embodiment 44: The pharmaceutical composition for use according to Embodiment 42 or Embodiment 43, wherein the use comprises administering the pharmaceutical composition to the human once every month, once every 2 months, once every 3 months, once every 4 months, once every 5 months, or once every 6 months.
Embodiment 45: The pharmaceutical composition for use in treatment of HIV according to Embodiment 38, wherein the pharmaceutical composition is administered in combination with one or more nucleoside reverse transcriptase inhibitors (NRTTIs), one or more non-nucleoside reverse transcriptase inhibitors (NNRTIs), one or more capsid inhibitors, and/or one or more broadly neutralizing antibodies (bnAbs).
Embodiment 46: A method for treating HIV in a human in need thereof comprising administering to said human a therapeutically effective amount of the pharmaceutical composition according to any one of Embodiments 1-41.
Embodiment 47: A method for preventing HIV in a human comprising administering to said human an effective amount of the pharmaceutical composition according to any one of Embodiments 1-41.
Embodiment 48: The method according to Embodiment 46 or Embodiment 47, wherein the method comprises a step of administering the pharmaceutical composition subcutaneously or intramuscularly.
Embodiment 49: The method according to any one of Embodiments 46-48, wherein about 300 mg to about 3200 mg of cabotegravir is administered to the human.
Embodiment 50: The method according to any one of Embodiments 46-49, wherein the method comprises administering the pharmaceutical composition to the human once every month, once every 2 months, once every 3 months, once every 4 months, once every 5 months, or once every 6 months.
Embodiment 51: The method according to any one of Embodiments 46-50, wherein the method comprises administering the pharmaceutical composition intramuscularly to a human once every 4 months.
Embodiment 52: The method according to any one of Embodiments 46-51, wherein the method comprises administering the pharmaceutical composition in combination with one or more nucleoside reverse transcriptase inhibitors (NRTTIs), one or more non-nucleoside reverse transcriptase inhibitors (NNRTI), one or more capsid inhibitors, and/or one or more broadly neutralizing antibodies (bnAbs).
Embodiment 53: A kit comprising a container that contains the pharmaceutical composition according to any one of Embodiments 1-45 as a lyophilized powder.
Embodiment 54: The kit according to Embodiment 53, wherein the kit further comprises a container comprising water.
Embodiment 55: A method of preparing a reconstituted solution, the method comprising providing the kit of Embodiment 53 and contacting the lyophilized composition with a suitable liquid to produce a reconstituted solution.
Embodiment 56: The method according to Embodiment 55, wherein the suitable liquid is an aqueous solvent.
Embodiment 57: The method according to Embodiment 55 or Embodiment 56, wherein the suitable liquid is water.
Embodiment 58: The method according to Embodiment 55, wherein the suitable liquid is a non-aqueous solvent.

### EXAMPLES

### Comparative Example (Example 8 of WO 2021/116872A1)

A long-acting suspension comprising 200 mg/mL cabotegravir, 20 mg/mL PS20 and 20 mg/mL PEG3350 was investigated to test stability when the cabotegravir concentration was increased to 400 mg/mL.

The suspension was prepared in a 250 mL batch using a Netzsch miniCer and 0.3 mm YTZ grinding beads. A range of excipient concentrations were explored.

The formulation vehicle was prepared by dissolving Polysorbate 20 (Croda), Polyethylene Glycol 3350 (Clariant), and mannitol (Roquette Freres) in water for injection (WFI) and filtering the solution through a 0.2 μm filter. The formulation vehicle was then added to cabotegravir (free acid) to prepare a 400 mg/mL coarse suspension. The coarse suspension, while stirred, was circulated through a wet bead mill set at 29.7 Hz (Netzsch MiniCer) containing 0.30 mm YTZ grinding beads (Nikkato Corp) at 73-145 mL/min until the desired median particle diameter of less than 0.25 μm was reached as measured by laser diffraction. The wet bead mill was cooled to maintain a temperature between 1 and 25° C. The suspension was then filled into Type I glass vials, flushed with nitrogen, stoppered (FM457 stopper) and sealed. The filled suspension was terminally sterilized by gamma irradiation at a minimum dose of 25 kGy.

30 mg/mL PS20, 20 mg/mL PEG3350 and 19 mg/mL mannitol with 400 mg/mL cabotegravir was prepared. The gamma irradiated suspension in vials was set down in the upright position and stored at 40° C./75% RH. After 1 month and 3 months of storage, suspensions were tested for suitability. Table 3 shows particle size (μm) after formulation and after 1 month of storage at 40° C./75% RH for the 400 mg/mL cabotegravir formulation with 30 mg/mL PS20, 30 mg/mL PEG3350, 19 mg/mL mannitol. At 1 month, the particle size had increased compared to the initial time point (Table 3). At 3 months, the suspension had become an unrecoverable gel which could not be removed from the vial with a syringe.

TABLE 3

| Time | X10 | X50 | X90 |
|---|---|---|---|
| Initial | 0.07 | 0.17 | 0.61 |
| 1 month | 0.14 | 0.41 | 3.2 |

Further suspensions comprising cabotegravir, PS20 and PEG3350 were prepared with the concentrations shown in Table 4. Experiments 1, 2, and 5 were prepared using the method described above for 20 mg/mL PS20 and PEG3350. In experiment 3, the suspension was first milled with PS20 (no PEG3350), and then PEG3350 was added, here 400 mg/mL cabotegravir was milled with 30 mg/mL PS20. After milling, a concentrated solution (400 mg/mL) of PEG3350 was added to dilute the 400 mg/mL suspension to 360 mg/mL cabotegravir, 27 mg/mL PS20, 27 mg/mL mannitol. Compositions were all prepared on a 250 mL batch scale using the Netzsch miniCer with 0.3 mm YTZ grinding beads.

TABLE 4

| Experiment Number | PS20 (mg/mL) | PEG3350 (mg/mL) |
|---|---|---|
| 1 | 40.0 | 40.0 |
| 2 | 30.0 | 40.0 |
| 3 | 27* | 40.0 |
| 4 | 20 | 20.0 |
| 5 | 20 | 0 |

*400 mg/mL was diluted down by 10%, resulting in 360 mg/mL cabotegravir and 27 mg/mL PS20

In experiments 1, 2, 4 and 5 during wet bead milling on the miniCer, the suspension thickened into a paste and was not recoverable. In experiment 3, within 5 minutes of adding the 400 mg/mL PEG3350 solution, the suspension thickened and could no longer be stirred. The combination of PS20 and PEG3350 with 400 mg/mL cabotegravir did not produce a physically stable suspension.

### Experimental Examples

In the following description of the Examples, specific embodiments are described. These embodiments are described in sufficient detail to enable those skilled in the art to practice certain embodiments of the present disclosure. Other embodiments may be utilized and logical and other changes may be made without departing from the scope of the disclosure. The following description is, therefore, not intended to limit the scope of the present disclosure.

Cabotegravir particle sizes in microsuspensions in the following examples were measured according to the below protocol description.

### Protocol Description

Carried out the method for Laser Diffraction Measurement of Particle Size (USP <429>).
Reconstituted the lyophilized product with water for injection (WFI) (volume of WFI determined by desired concentration of reconstituted suspension)
Cleaned and filled Malvern Mastersizer 3000 laser diffraction with MV instrument with deionized water.
Dispersion stirring speed was 2000 rpm, particle refractive index was 1.67, particle absorption Index (imaginary part of refractive index) was 0.01, General Purpose analysis mode with normal sensitivity, dispersant (DI water) refractive index was 1.33, measurement time and background time were both 10 seconds, and 2 measurements were acquired by the instrument per aliquot.
Performed an alignment and measured the background.
Agitated suspension (created in below examples) to ensure that all particles are suspended in the drug product vial.
Withdrew ˜0.2 mL of content with a 1 mL syringe fitted with a needle (18 G).
Added two drops of the suspension into a microcentrifuge tube containing 0.5 mL of 6.6% w/v P338 in water solution.
Vortexed gently to create a homogenous dispersion of drug product in P338 dispersant.
Added appropriate amount of this dispersion to the instrument until obscuration of 5%-7% is achieved.
Allowed test suspension to circulate in the instrument for approximately 30 seconds prior to initiating the measurement.
Performed the measurement, and after each measurement flushed the instrument with deionized water.
Repeated this three times using a new aliquot of the sample each time and calculated the average values obtained from the volume distribution.

### Example 1

TABLE 5

| Ingredient | Concentration (mg/mL) |
|---|---|
| Cabotegravir, micronized | 400.0 |
| Sodium Carboxymethylcellulose | 3.7 |
| Polysorbate 80 | 3.0 |
| Mannitol | 25.9 |

Table 5 shows an exemplary pharmaceutical composition of the invention (pharmaceutical compositions are also described in these examples as “suspensions”), which was made using the following method.

A formulation vehicle was prepared by dissolving/diluting 24.0 g polysorbate 80 (“PS80”) (Croda) in about 300 g water. Separately, 30.0 g sodium carboxymethylcellulose (“NaCMC”) (Ashland, 7L2P) and 210.0 g mannitol (Roquette Freres) were dissolved in 4.8 kg water for injection (WFI). Once the NaCMC and mannitol were dissolved, the PS80 solution was added to the NaCMC-mannitol solution with stirring. The weighing and dilution vessels were rinsed into the compounding vessel with additional water and the compounded vehicle solution was brought to a final weight of 6.06 kg and filtered through a 0.2 μm filter. 1.6 kg Cabotegravir Micronized Free Acid (target X50=5-6 μm particle size) was added to 3.0 kg filtered vehicle and was mixed to form a homogeneous suspension. The compounded suspension was deaerated while stirring until it reached its target batch volume, and the suspension was then filled into vials. The product was lyophilized by freezing at −45° C. for at least 2 hours, annealing at −18° C. for at least 2 hours, refreezing at −45° C. for at least 2 hours (each transition at a ramp rate of +/−1° C./min), primary drying at −10° C. (ramp rate: 0.15° C./min) for at least 23 hours at approximately 150 mTorr, and secondary drying at 25° C. (ramp rate: 0.58° C./min) for at least 6 hours at approximately 150 mTorr. The lyophilized vials were backflushed with nitrogen to about 600 Torr, sealed, and sterilized by gamma irradiation at a minimum dose of 25 kGy. The formulation is reconstituted with WFI and briefly shaken to resuspend prior to administration.

### Example 2

TABLE 6

| Ingredient | Concentration (mg/mL) |
|---|---|
| Cabotegravir, micronized | 400.0 |
| Sodium Carboxymethylcellulose | 5.0 |
| Polysorbate 80 | 4.0 |
| Mannitol | 35.0 |

Table 6 shows an exemplary pharmaceutical composition of the invention (pharmaceutical compositions are also described in these examples as “suspensions”), which was made using the following method.

A formulation vehicle was prepared by dissolving/diluting 24.0 g polysorbate 80 (“PS80”) (Croda) in about 300 g water. Separately, 30.0 g sodium carboxymethylcellulose (“NaCMC”) (Ashland, 7L2P) and 210.0 g mannitol (Roquette Freres) were dissolved in 3.4 kg water for injection (WFI). Once the NaCMC and mannitol were dissolved, the PS80 solution was added to the NaCMC-mannitol solution with stirring. The weighing and dilution vessels were rinsed into the compounding vessel with additional water and the compounded vehicle solution was brought to a final weight of 4.50 kg and filtered through a 0.2 μm filter. 1.6 kg Cabotegravir Micronized Free Acid (target X50=5-6 μm particle size) was added to 3.0 kg filtered vehicle and was mixed to form a homogeneous suspension. The compounded suspension was deaerated while stirring until it reached its target batch volume, and the suspension was then filled into vials. The product was lyophilized by freezing at −45° C. for at least 2 hours, annealing at −10° C. for at least 2 hours, refreezing at −45° C. for at least 2 hours (each transition at a ramp rate of +/−1° C./min), primary drying at −5° C. (ramp rate: 1° C./min) for at least 20 hours at approximately 150 mTorr, and secondary drying at 40° C. (ramp rate: 1° C./min) for at least 6 hours at approximately 150 mTorr. The lyophilized vials were backflushed with nitrogen to about 600 Torr, sealed, and sterilized by gamma irradiation at a minimum dose of 25 kGy. The formulation is reconstituted with WFI and briefly shaken to resuspend prior to administration.

### Example 3

TABLE 7

| Ingredient | Concentration (mg/mL) |
|---|---|
| Cabotegravir, micronized | 400.0 |
| Sodium Carboxymethylcellulose | 5.0 |
| Polysorbate 80 | 4.0 |
| Mannitol | 35.0 |

Table 7 shows an exemplary pharmaceutical composition of the invention (pharmaceutical compositions are also described in these examples as “suspensions”), which was made using the following method.

A formulation vehicle was prepared by dissolving/diluting 24.0 g polysorbate 80 (“PS80”) (Croda) in about 300 g water. Separately, 30.0 g sodium carboxymethylcellulose (‘NaCMC’) (Ashland, 7L2P) and 210.0 g mannitol (Roquette Freres) were dissolved in 3.4 kg water for injection (WFI). Once the NaCMC and mannitol were dissolved, the PS80 solution was added to the NaCMC-mannitol solution with stirring. The weighing and dilution vessels were rinsed into the compounding vessel with additional water and the compounded vehicle solution was brought to a final weight of 4.50 kg and filtered through a 0.2 μm filter. 1.6 kg Cabotegravir Micronized Free Acid (target X50=3-4 μm particle size) was added to 3.0 kg filtered vehicle and was mixed to form a homogeneous suspension. The compounded suspension was deaerated while stirring until it reached its target batch volume, and the suspension was then filled into vials. The product was lyophilized by freezing at −45° C. for at least 2 hours, annealing at −10° C. for at least 2 hours, refreezing at −45° C. for at least 2 hours (each transition at a ramp rate of +/−1° C./min), primary drying at −5° C. (ramp rate: 1° C./min) for at least 20 hours at approximately 150 mTorr, and secondary drying at 40° C. (ramp rate: 1° C./min) for at least 6 hours at approximately 150 mTorr. The lyophilized vials were backflushed with nitrogen to about 600 Torr, sealed, and sterilized by gamma irradiation at a minimum dose of 25 kGy. The formulation was reconstituted with WFI and briefly shaken to resuspend prior to administration.

Table 8, below, applies to Examples 4-6.

TABLE 8

| Batch # | Formulation (when reconstituted) |
|---|---|
| 1 | Cabotegravir 400 mg/mL; 0.4 w/v % (4 mg/mL) PS80; |
| | 0.5 w/v % (5 mg/mL) NaCMC; 3.5 w/v % (35 mg/mL) |
| | Mannitol |
| 2 | Cabotegravir 400 mg/mL; 0.4 w/v % (4 mg/mL) PS20; |
| | 0.5 w/v % (5 mg/mL) NaCMC; 3.5 w/v % (35 mg/mL) |
| | Mannitol |
| 3 | Cabotegravir 400 mg/mL; 0.4 w/v % (4 mg/mL) P338; |
| | 0.5 w/v % (5 mg/mL) NaCMC; 3.5 w/v % (35 mg/mL) |
| | Mannitol |

### Example 4: Stability of a Composition Comprising Cabotegravir, Sodium Carboxymethylcellulose, Polysorbate 80 and Mannitol

A formulation vehicle was prepared by dissolving 2.10 g PS80 (Croda), 2.63 g NaCMC (Ashland), and 18.38 g mannitol (Roquette Freres) in 369.6 g WFI and filtering the solution through a 0.2 μm filter. The formulation vehicle was added to 210 g Cabotegravir (free acid) to prepare a 400 mg/mL coarse suspension. The suspension was covered and stirred for 2 hrs. The suspension was filled into Type I glass vials and lyophilized as described in Examples 1-3. The lyophilized suspension was reconstituted to a cabotegravir concentration of 400 mg/mL.

TABLE 9
Batch #1 (400 mg/mL cabotegravir; 0.4 w/v
% PS80; 0.5 w/v % NaCMC; 3.5 w/v % mannitol)

| Storage | Time | Particle Size by Laser Diffraction (microns) | | |
|---|---|---|---|---|
| Condition | (Months) | X10 | X50 | X90 |
| Initial | 0 | 3.1 | 7.6 | 15.6 |
| 30° C./75% | 1 | 3.2 | 7.7 | 15.4 |
| RH Upright | 3 | 3.2 | 7.5 | 14.3 |
| | 6 | 3.2 | 7.6 | 15.3 |
| | 9 | 3.2 | 7.5 | 14.8 |
| | 12 | 3.3 | 7.5 | 14.3 |
| 40° C./75% | 1 | 3.2 | 7.6 | 15.0 |
| RH Upright | 3 | 3.2 | 7.7 | 14.9 |
| | 6 | 3.1 | 7.3 | 13.9 |
| 50° C./AmbH | DY14 | 3.2 | 7.7 | 15.7 |
| Upright | 1 | 3.2 | 7.6 | 15.3 |
| | 3 | 3.3 | 7.7 | 14.7 |

Notes:
AmbH Ambient Humidity
RH Relative Humidity
— Denotes testing not scheduled at these timepoints
NGT = Not greater than
NLT = Not less than

### Example 5: Stability of a Composition Comprising Cabotegravir, Sodium Carboxymethylcellulose, Polysorbate 20 and Mannitol

A formulation vehicle was prepared by dissolving 2.10 g polysorbate 20 (Croda), 2.63 g sodium CMC (Ashland), and 18.38 g mannitol (Roquette Freres) in 550.9 g WFI and filtering the solution through a 0.2 μm filter. The formulation vehicle was added to 210 g cabotegravir (free acid) to prepare a 300 mg/mL coarse suspension. The suspension was covered and stirred for 2 hrs. The suspension was filled into Type I glass vials and lyophilized as described in Examples 1-3. The lyophilized suspension was reconstituted to a cabotegravir concentration of 400 mg/mL.

TABLE 10
Batch #2 (400 mg/mL cabotegravir; 0.4 w/v
% PS20; 0.5 w/v % NaCMC; 3.5 w/v % mannitol)

| Storage | Time | Particle Size by Laser Diffraction (microns) | | |
|---|---|---|---|---|
| Condition | (Months) | X10 | X50 | X90 |
| Initial | 0 | 3.0 | 7.4 | 14.9 |
| 30° C./75% | 1 | 3.0 | 7.4 | 14.8 |
| RH Upright | 3 | 3.0 | 7.4 | 15.0 |
| | 6 | 2.9 | 7.0 | 13.8 |
| | 9 | 3.0 | 7.3 | 14.0 |
| | 12 | 3.2 | 7.4 | 14.0 |
| 40° C./75% | 1 | 2.8 | 7.1 | 14.2 |
| RH Upright | 3 | 2.9 | 7.3 | 14.6 |
| | 6 | 2.9 | 7.1 | 14.4 |
| 50° C./AmbH | DY14 | 2.8 | 7.2 | 14.7 |
| Upright | 1 | 2.9 | 7.3 | 14.3 |
| | 3 | 3.0 | 7.4 | 14.4 |

Notes:
AmbH Ambient Humidity
RH Relative Humidity
— Denotes testing not scheduled at these timepoints
NGT = Not greater than
NLT = Not less than

### Example 6: Stability of a Composition Comprising Cabotegravir, Sodium Carboxymethylcellulose, Poloxamer 338 and Mannitol

A formulation vehicle was prepared by dissolving 2.10 g poloxamer 338 (BASF), 2.63 g sodium CMC (Ashland), and 18.38 g mannitol (Roquette Freres) in 369.6 g WFI and filtering the solution through a 0.2 μm filter. The formulation vehicle was added to 210 g Cabotegravir (free acid) to prepare a 400 mg/mL coarse suspension. The suspension was covered and stirred for 2 hrs. The suspension was filled into Type I glass vials and lyophilized as described in Examples 1-3. The lyophilized suspension was reconstituted to a cabotegravir concentration of 400 mg/mL.

TABLE 11
Batch #3 (400 mg/mL cabotegravir; 0.4 w/v
% P338; 0.5 w/v % NaCMC; 3.5 w/v % mannitol)

| Storage | Time | Particle Size by Laser Diffraction (microns) | | |
|---|---|---|---|---|
| Condition | (Months) | X10 | X50 | X90 |
| Initial | 0 | 3.4 | 7.8 | 15.2 |
| 30° C./75% | 1 | 3.3 | 7.6 | 14.6 |
| RH Upright | 3 | 3.3 | 7.7 | 15.3 |
| | 6 | 3.2 | 7.3 | 13.4 |
| | 9 | 3.3 | 7.8 | 15.3 |
| | 12 | 3.3 | 7.5 | 13.9 |
| 40° C./75% | 1 | 3.5 | 7.8 | 14.8 |
| RH Upright | 3 | 3.2 | 7.7 | 15.4 |
| | 6 | 3.2 | 7.6 | 15.6 |
| 50° C./AmbH | DY14 | 3.3 | 7.7 | 14.8 |
| Upright | 1 | 3.3 | 7.6 | 14.3 |
| | 3 | 3.3 | 7.5 | 14.5 |

Notes:
AmbH Ambient Humidity
RH Relative Humidity
— Denotes testing not scheduled at these timepoints
NGT = Not greater than
NLT = Not less than

### Example 7: Stability of a Composition Comprising Cabotegravir, Sodium Carboxymethylcellulose, Polysorbate 80 and Mannitol

A formulation vehicle was prepared by dissolving 1.04 g PS80 (Croda), 1.30 g NaCMC (Ashland; 7LF), and 9.1 g mannitol (Roquette Freres) in 183.4 g WFI. 195.0 mL of the formulation vehicle was added to 104.0 g Cabotegravir (free acid) to prepare a 400 mg/mL coarse suspension. The suspension was covered and stirred for 2 hrs. The suspension was filled into Type I glass vials and lyophilized as described in Examples 1-3. The lyophilized suspension was reconstituted to a cabotegravir concentration of 533 mg/mL.

TABLE 12
Batch #1 (533 mg/mL cabotegravir; 0.53 w/v
% PS80; 0.67 w/v % NaCMC; 4.66 w/v % mannitol)

| Storage | Time | Particle Size by Laser Diffraction (microns) | | |
|---|---|---|---|---|
| Condition | (Months) | X10 | X50 | X90 |
| Initial | 0 | 1.9 | 5.0 | 11.8 |
| 50° C./AmbH | 1 | 1.9 | 4.9 | 11.7 |
| Upright | | | | |

Notes:
AmbH Ambient Humidity
RH Relative Humidity
— Denotes testing not scheduled at these timepoints
NGT = Not greater than
NLT = Not less than

### Example 8: In Vivo Rat Pharmacokinetic Study for Compositions Comprising Cabotegravir, Sodium Carboxymethylcellulose, Mannitol and Either Polysorbate 80 or Poloxamer 338

Composition preparation was described in Examples 4 and 6. Formulations were dosed in 9 Sprague Dawley male rats at 30 mg/kg in the subcutaneous space of the intrascapular region. Briefly, two nanosuspension formulations of Cabotegravir at 200 (group 1) and 400 mg/mL (group 2), also differing in excipient composition, were administered subcutaneously through a single injection at target dose of 30 mg/kg in each rat. Similarly, two lyophilized powder formulations of Cabotegravir at micron-size, differing in excipient composition, were reconstituted with WFI to 400 mg/mL concentration and administered subcutaneously through a single injection at target dose of 30 mg/kg in each rat. Details of the formulations are described in Table 13.

TABLE 13
Suspension formulations details for
test articles dosed in animal PK study

| | | Particle size | | | |
|---|---|---|---|---|---|
| | Cabotegravir | distribution | | | |
| | Concentration | (μm) | | | Formulation |
| Group | (mg/mL) | ×10 | ×50 | ×90 | composition |
| 1 | 200.6 | 0.10 | 0.23 | 0.60 | 2 w/v % |
| | | | | | (20 mg/mL) |
| | | | | | Polysorbate |
| | | | | | 20, 2 w/v % |
| | | | | | (20 mg/mL) |
| | | | | | Polyethylene |
| | | | | | Glycol 3350, |
| | | | | | 3.5 w/v % |
| | | | | | (35 mg/mL) |
| | | | | | Mannitol |
| 2 | 399.6 | 0.06 | 0.15 | 0.56 | 3.8 w/v % |
| | | | | | (38 mg/mL) |
| | | | | | Poloxamer |
| | | | | | 338, 3.5 w/v % |
| | | | | | (35 mg/mL) |
| | | | | | Polyethylene |
| | | | | | Glycol 3350 and |
| | | | | | 2 w/v % |
| | | | | | (20 mg/mL) |
| | | | | | Mannitol |
| 3 | 400 | 3.1 | 7.6 | 15.6 | 0.4 w/v % |
| | | | | | (4 mg/mL) |
| | | | | | Polysorbate |
| | | | | | 80, 0.5 w/v % |
| | | | | | (5 mg/mL) Sodium |
| | | | | | Carboxymethyl- |
| | | | | | cellulose and 3.5 |
| | | | | | (3 mg/mL) |
| | | | | | w/v % Mannitol |
| 4 | 400 | 3.0 | 7.4 | 14.9 | 0.4 w/v % |
| | | | | | (4 mg/mL) |
| | | | | | Poloxamer |
| | | | | | 338, 0.5 w/v % |
| | | | | | (5 mg/ml) Sodium |
| | | | | | Carboxymethyl- |
| | | | | | cellulose and |
| | | | | | 3.5 w/v % |
| | | | | | (35 mg/mL) |
| | | | | | Mannitol |

Table 14 shows Cabotegravir plasma pharmacokinetics for a preclinical study and statistical analysis through t-test for several pharmacokinetic parameters. The lyophilized formulation from Group 3 highlights a significantly reduced Cₘₐₓ (1.8 folds) and extended t_{1/2} (1.5 folds) compared to the nanosuspension formulation from Group 1. In addition, the lyophilized formulation from Group 3 demonstrates improved t_{1/2} compared to formulation from Group 4.

TABLE 14
Cabotegravir pharmacokinetic parameters for PK study
in example 8 and statistical analysis through t-test

| | | Cₘₐₓ | Tₘₐₓ | t_{1/2} | MRT_{inf} | AUCₗₐₛₜ | AUC_{inf} | Lambda_z |
|---|---|---|---|---|---|---|---|---|
| | | (ng/mL) | (h) | (h) | (h) | (h*μg/mL) | (h*μg/mL) | (1/min) |
| Group | Mean | 55250 | 360 | 462 | 859 | 48559 | 52816 | 2.74E−05 |
| 1 | SD | 12418 | 57 | 151 | 233 | 12414 | 14441 | 9.21E−06 |
| Group | Mean | 43000 | 280 | 280 | 550 | 30193 | 31289 | 4.90E−05 |
| 2 | SD | 29000 | 120 | 140 | 200 | 20721 | 22053 | 1.80E−05 |
| Group | Mean | 31000 | 470 | 700 | 1300 | 37410 | 45784 | 1.90E−05 |
| 3 | SD | 11000 | 110 | 270 | 270 | 12207 | 14775 | 9.10E−06 |
| Group | Mean | 40000 | 450 | 420 | 820 | 37126 | 39373 | 3.20E−05 |
| 4 | SD | 16000 | 120 | 160 | 170 | 16465 | 17500 | 1.40E−05 |
| p | G3 vs | 0.069 | 0.371 | ** | *** | 0.484 | 0.207 | * |
| value | G4 | | | | | | | |
| | G3 vs | *** | * | * | ** | * | 0.169 | * |
| | G1 | | | | | | | |
| | G3 vs | 0.127 | ** | *** | **** | 0.191 | 0.0604 | *** |
| | G2 | | | | | | | |

^{#} Data from 8 animals (1 animal excluded as outlier)
p value calculated from one-tailed unpaired t-test for significance: * = 0.01 < p < 0.05; ** = 0.001 < p < 0.01; *** = 0.0001 < p < 0.001; p < 0.0001

Tolerability, with particular focus on injection site reaction, was assessed as part of the study and results are summarized in table 15. Occurrence of edema and scab formation were minimal and observed in few animals for a short timeframe. Nonetheless, minor differences were recorded among the 4 groups, with Groups 3 and 4 producing a lower frequency of edema, particularly Group 3.

TABLE 15
Injection site reaction observations

| | Edema | Scab formation |
|---|---|---|
| Group | Very slight in 6/9 rats - | Slight in 2/9 rats - days |
| 1 | days 4-22 | 11-43 |
| Group | Very slight in 6/9 rats - | Slight in 2/9 rats - days |
| 2 | days 3-57 | 11-15 |
| Group | Very slight in 2/9 rats - | Slight in 2/9 rats - days |
| 3 | days 6-8 | 11-57 |
| Group | Very slight in 4/9 rats | Slight in 3/9 rats - days |
| 4 | days 4-15 | 29-43 |

### Example 9

Example 9 evaluated the safety, tolerability and pharmacokinetics of single-dose administration of a pharmaceutical composition of the invention in 57 healthy adult participants. The lyophilized formulations of Example 9 are provided in Tables 16a and 16b below.

TABLE 16a

| Formulation Components | Cohorts C1 and C2 |
|---|---|
| Component | Quantity (mg/vial) |
| Cabotegravir | 800 |
| Mannitol | 51.8 |
| Polysorbate 80 | 5.9 |
| Sodium Carboxymethylcellulose | 7.4 |
| Water for injection* | q.s. |
| Nitrogen** | q.s. |

*Water is removed during manufacturing process.
**Nitrogen is utilized as a processing aid during vial stoppering.

TABLE 16b

| Formulation Components | Cohorts C3 - C6 |
|---|---|
| Component | Quantity (mg/vial) |
| Cabotegravir | 800 |
| Mannitol | 70 |
| Polysorbate 80 | 8 |
| Sodium Carboxymethylcellulose | 10 |
| Water for injection* | q.s. |
| Nitrogen** | q.s. |

*Water is removed during manufacturing process.
**Nitrogen is utilized as a processing aid during vial stoppering.

Cabotegravir particle size by diffraction (micron) is described in Table 17 below:

TABLE 17

| Quantile | Micron (μm) |
|---|---|
| X10 | 1.4-3.3 |
| X50 | 3.4-7.5 |
| X90 | 7.6-21.3 |

Vials containing the formulation of Table 16a were reconstituted with 1.7 mL water to achieve suspensions having a cabotegravir concentration of 400 mg/mL. Doses of the formulation of Table 16a were administered in two cohorts: 800 mg (2 ml of 400 mg/mL suspension) via the subcutaneous (SC) abdominal route (Cohort C1; 8 participants) and via the intramuscular (IM) (gluteus medius) route (Cohort C2; 8 participants).

Vials containing the formulation of Table 16b were reconstituted with either 1.7 mL water to achieve suspensions having a cabotegravir concentration of 400 mg/mL or with 1.1 mL water to achieve suspensions having a cabotegravir concentration of 533 mg/mL. Doses of the formulation of Table 16b were administered in three cohorts: 1200 mg (3 mL of 400 mg/mL suspension) via the subcutaneous (SC) abdominal route (Cohort C3; 8 participants), 1200 mg (3 mL of 400 mg/mL suspension) via the intramuscular (IM) (gluteus medius) route (Cohort C4; 8 participants), 1600 mg (3 mL of 533 mg/mL suspension) via the intramuscular (IM) (gluteus medius) route (Cohort C5; 16 participants), 2400 mg (4.5 mL of 533 mg/mL suspension administered as 2.2 mL in one gluteus medius muscle (left or right) and 2.3 mL in the other gluteus medius muscle (right or left) via the IM route (Cohort C6; 9 participants), and 3200 mg (6 mL of 533 mg/mL suspension administered as 3 mL in each gluteus medius muscle (left and right, respectively) via the IM route (Cohort C7; 7 participants).

### Safety and Tolerability

The safety and tolerability profile of the pharmaceutical composition of Tables 16a and 16b dosed in Example 9 was acceptable. Adverse events (AEs) occurred in 63-100% of participants (Table 18). Injection site reactions (ISRs) were the most common AEs and were mostly Grade 1 (Table 19). Overall, fewer ISRs were reported following IM administration compared to SC administration; however, ISRs were predominantly Grade (Table 19). No Grade 4 or serious AEs have been reported in Cohorts C1 to C7.

TABLE 18
Summary of most frequent (≥2 participants across cohorts)
AEs (ISR and non-ISR) by cohort

| | Cohort | Cohort | Cohort | Cohort | Cohort | Cohort | Cohort |
|---|---|---|---|---|---|---|---|
| | C1 | C2 | C3 | C4 | C5 | C6 | C7 |
| | 800 mg | 800 | 1200 | 1200 | 1600 | 2400 | 3200 |
| Most Frequent | SC | mg IM | mg SC | mg IM | mg IM | mg IM | mg IM |
| Adverse Events | (N = 8) | (N = 8) | (N = 8) | (N = 8) | (N = 16) | (N = 9) | (N = 7) |
| Any event, n (%) | 8 (100) | 5 (63) | 8 (100) | 8 (100) | 12 (75) | 9 (100) | 7 (100) |
| Any ISR, n (%) | 8 (100) | 3 (38) | 8 (100) | 8 (100) | 11 (69) | 8 (89) | 7 (100) |
| ISRs | | | | | | | |
| Injection site erythema | 8 (100) | 0 | 7 (88) | 0 | 1 (6) | 1 (11) | 0 |
| Injection site pain | 3 (38) | 2 (25) | 5 (63) | 8 (100) | 8 (50) | 7 (78) | 7 (100) |
| Injection site nodule | 6 (75) | 0 | 8 (100) | 0 | 2 (13) | 5 (56) | 1 (14) |
| Injection site swelling | 3 (38) | 2 (25) | 2 (25) | 0 | 3 (19) | 2 (22) | 1 (14) |
| Injection site pruritus | 0 | 0 | 1 (13) | 0 | 0 | 2 (22) | 1 (14) |
| Non-ISRs | | | | | | | |
| Nasopharyngitis/ | 1 (13) | 0 | 0 | 0 | 1 (6) | 0 | 0 |
| pharyngitis | | | | | | | |
| streptococcal | | | | | | | |
| (Viral) upper | 1 (13) | 0 | 2 (25) | 0 | 0 | 0 | 0 |
| respiratory tract | | | | | | | |
| infection | | | | | | | |
| Urinary tract infection | 0 | 0 | 1 (13) | 0 | 0 | 1 (11) | 0 |
| Abdominal | 0 | 2 (25) | 1 (13) | 0 | 1 (6) | 0 | 0 |
| discomfort/abdominal | | | | | | | |
| pain lower/abdominal | | | | | | | |
| pain upper | | | | | | | |
| Diarrhoea | 0 | 1 (13) | 0 | 0 | 1 (6) | 1 (11) | 0 |
| Nausea | 0 | 0 | 0 | 0 | 1 (6) | 0 | 1 (14) |
| Dizziness | | 0 | 1 (13) | 1 (13) | 0 | 0 | 1 (14) |
| Headache | | 1 (13) | 1 (13) | 2 (25) | 0 | 1 (11) | 1 (14) |
| Back pain | | 0 | 0 | 0 | 2 (13) | 0 | 0 |
| Cough | | 1 (13) | 0 | 0 | 1 (6) | 0 | 1 (14) |
| Pollakiuria | 10 | 0 | 0 | 0 | 2 (13) | 0 | 0 |
| Blood creatine | 0 | 0 | 0 | 1 (13) | 0 | 1 (11) | 0 |
| phosphokinase | | | | | | | |
| increased | | | | | | | |

TABLE 19
Summary of ISRs

| | Cohort | Cohort | Cohort | Cohort | Cohort | Cohort | Cohort |
|---|---|---|---|---|---|---|---|
| | C1 | C2 | C3 | C4 | C5 | C6 | C7 |
| | SC | IM | SC | IM | IM | IM | IM |
| | 800 mg | 800 mg | 1200 mg | 1200 mg | 1600 mg | 2400 mg | 3200 mg |
| | [2 mL] | [2 mL] | [3 mL] | [3 mL] | [3 mL] | [4.5 mL] | [6 mL] |
| PARAMETER, N | (n = 8) | (n = 8) | (n = 8) | (n = 8) | (n = 16) | (n = 9) | (n = 7) |
| (% OF PARTICIPANTS) | Single injection | | | | | Two injections | |
| Any ISR | 8 (100) | 3 (38) | 8 (100) | 8 (100) | 11 (69) | 8 (89) | 7 (100) |
| Drug-related | 8 (100) | 3 (38) | 8 (100) | 8 (100) | 10 (63) | 8 (89) | 7 (100) |
| Serious | 0 | 0 | 0 | 0 | 0 | 0 | 0 |
| Grade 3 | 0 | 0 | | 0 | 0 | | 0 |
| Grade 4 | 0 | 0 | 0 | 0 | 0 | 0 | 0 |
| Pain | 3 (38) | 2 (25) | 5 (63) | 8 (100) | 8 (50) | 7 (78) | 7 (100) |
| Grade 1 | (38) | 1 (13) | 5 (63) | 8 (100) | 8 (50) | 7 (78) | 5 (71) |
| Grade 2 | 0 | 1 (13) | 0 | 0 | 0 | 0 | 2 (29) |
| Grade 3 | 0 | 0 | 0 | 0 | 0 | 0 | 0 |
| Erythema | 8 (100) | 0 | 7 (88) | 0 | 1 (6) | 1 (11) | 0 |
| Grade 1 | 6 (75) | 0 | 5 (63) | 0 | 1 (6) | 0 | 0 |
| Grade 2 | 2 (25) | 0 | 2 (25) | 0 | 0 | 0 | 0 |
| Grade 3 | 0 | 0 | 0 | 0 | 0 | 1 (11) | 0 |
| Swelling/induration | 3 (38) | 2 (25) | 2 (25) | 0 | 3 (19) | 2 (22) | 1 (14) |
| Grade 1 | 3 (38) | 2 (25) | 2 (25) | 0 | 2 (13) | 1 (11) | 1 (14) |
| Grade 2 | 0 | 0 | 0 | 0 | 1 (6) | 1 (11) | 0 |
| Grade 3 | 0 | | | | | 0 | 0 |
| Nodule | 6 (75) | 0 | 8 (100) | 0 | 2 (13) | 5 (56) | 1 (14) |
| Grade 1 | 6 (75) | 0 | 8 (100) | 0 | 2 (13) | 5 (56) | 1 (14) |
| Grade 2 | 0 | 0 | 0 | 0 | 0 | 0 | 0 |
| Grade 3 | 0 | 0 | 0 | 0 | 0 | 0 | 0 |
| Total ISRs | 21 | 5 | 24 | 9 | 15 | 33 | 21 |
| (% of total ISRs) | | | | | | | |
| Grade 1 | 19 (90) | 4 (80) | 22 (92) | 9 (100) | 14 (93) | 29 (88) | 17 (81) |
| Grade 2 | 2 (10) | 1 (20) | 2 (8) | 0 | 1 (7) | 3 (9) | 4 (19) |
| Grade 3 | 0 | | | | | 1 (3) | 0 |
| Grade 4 | 0 | | | | | 0 | 0 |
| Median ISR duration | | | | | | | |
| (IQR), days [n = events | | | | | | | |
| with non-missing end | | | | | | | |
| date/total number | | | | | | | |
| of events] ^{a} | | | | | | | |
| Pain | n = 3/3 | n = 2/2 | n = 6/6 | n = 8/8 | n = 8/8 | n = 13/13 | n = 14/14 |
| | 7 | 6.5 | 8 | 4.5 | 6 | 7 | 6.5 |
| | (6-12) | (5-8) | (6-15) | (3.5-6.5) | (4-7) | (6-10) | (4-10) |
| Erythema | n = 8/8 | N/A | n = 7/7 | N/A | n = 1/1 | n = 2/2 | N/A |
| | 18 | | 15 | | 4 | 5 | |
| | (10-41) | | (13-31) | | (4-4) | (5-5) | |
| Swelling/induration | n = 3/3 | n = 3/3 | n = 2/2 | N/A | n = 3/3 | n = 4/4 | n = 2/2 |
| | 42 | 5 | 1.5 | | 5 | 38.5 | 2 |
| | (13-63) | (4-43) | (1-2) | | (5-19) | (5-72) | (2-2) |
| Nodule | n = 0/6 | N/A | n = 1/8 | N/A | n = 1/2 | n = 7/9 | n = 1/1 |
| | | | 106 | | 43 | 55 | 10 |
| | | | (106-106) | | (43-43) | (19-106) | (10-10) |

^{a} ISR duration is not calculated if end date is missing.

### Example 10

### PK and Simulations

Cabotegravir plasma Cmax following a single SC (abdominal) or IM (gluteal) injection of the pharmaceutical compositions described in Example 9 cohorts 1 to 5 was lower than that following a single IM injection of APRETUDE®, while plasma Cmax following bilateral IM gluteal injections (2.3 mL in one gluteus medius muscle (left or right) and 2.2 mL in the other gluteus medius muscle (right or left); 4.5 mL total) was comparable to that following a single IM injection of APRETUDE®. Based on observed PK data for cohorts 1 to 5 in Example 9, t1/2 following these cohorts SC and IM injection is predicted to be >6× (SC injections) and >2× (IM injections) the t_{1/2} of APRETUDE® (Q2M cabotegravir 200 mg/mL), respectively. There are not enough data to determine the t_{1/2} following administration of the cabotegravir pharmaceutical composition described in Example 9 cohorts 6 or 7.

TABLE 20
Plasma Pharmacokinetic and Safety Results in
Healthy Adult Participants Cohorts C1-C7

| | Cohort C1 | Cohort C2 | Cohort C3 | Cohort C4 | Cohort C5 | Cohort C6 | Cohort C7 |
|---|---|---|---|---|---|---|---|
| | (2 mL) SC | (2 mL) IM | (3 mL) SC | (3 mL) IM | (3 mL) IM | (4.5 mL) IM | (6 mL) IM |
| Parameter | (N = 8) | (N = 8) | (N = 8) | (N = 8) | (N = 16) | (N = 9) | (N = 7) |
| Cmax | 0.7 (35.1) | 1.8 (43.0) | 0.9 (32.6) | 1.8 (130.7) | 1.9 (85.3) | 4.1 (40.4) | * |
| μg/mL^{a} | | | | | | | |
| Grade 1 | 6 (75) | 2 (25) | 6 (75) | 8 (100) | 10 (63) | 7 (78) | 5 (71) |
| ISRs, n (%) | | | | | | | |
| Grade 2 | 2 (25) | 1 (13) | 2 (25) | 0 | 1 (6) | 0 | 2 (29) |
| ISRs, n (%) | | | | | | | |
| Grade ≥ 3 | 0 | 0 | 0 | 0 | 0 | 1 (11) | 0 |
| ISRs, n (%) | | | | | | | |

^{a}Value reported as geometric mean (% CVb).
* not yet calculable.

### Simulations

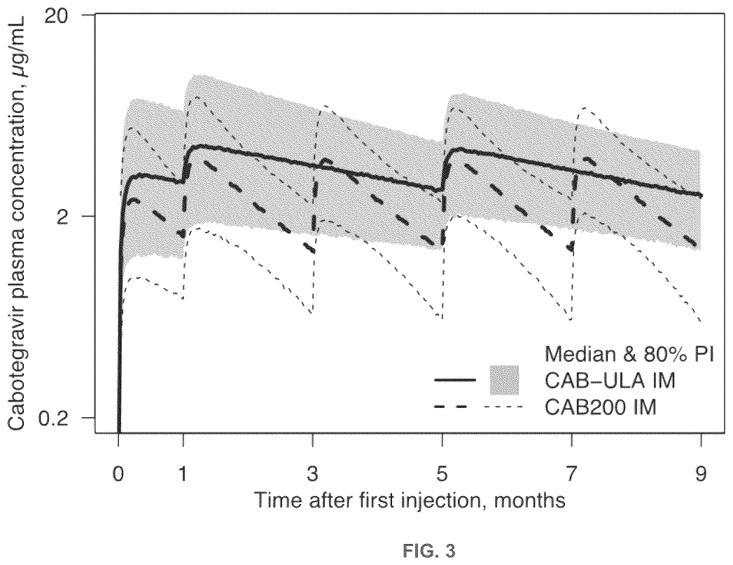

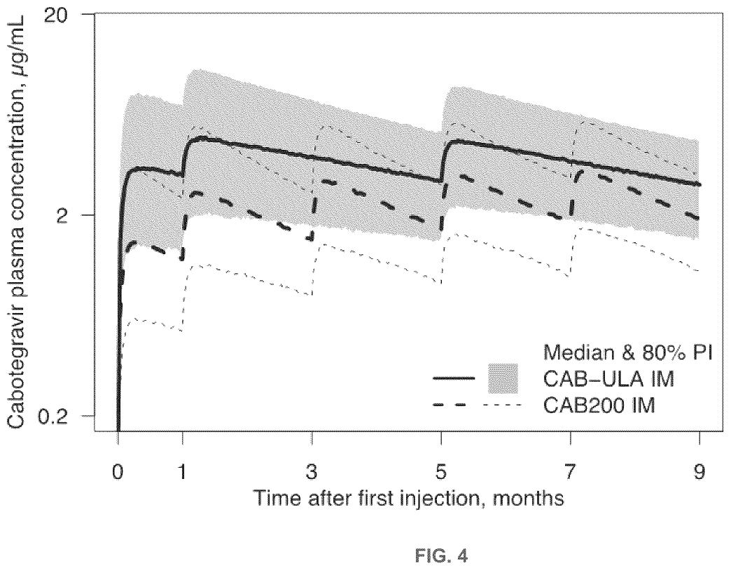

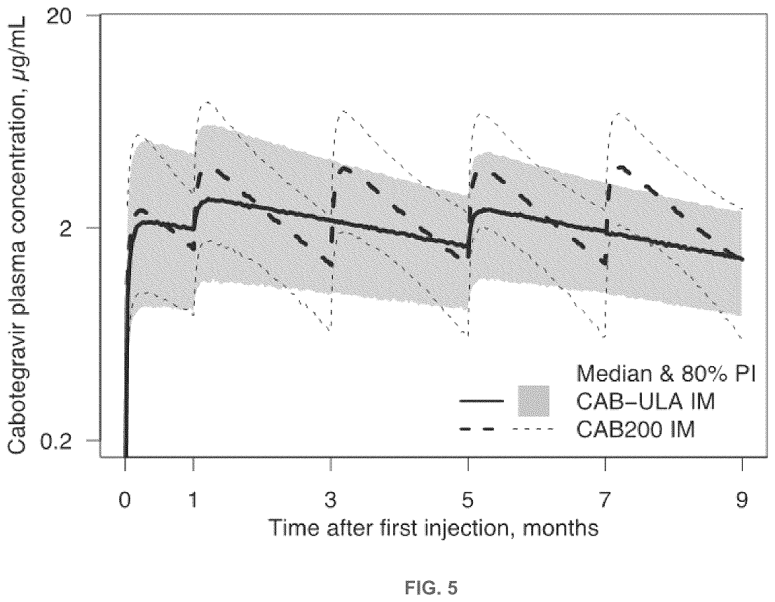

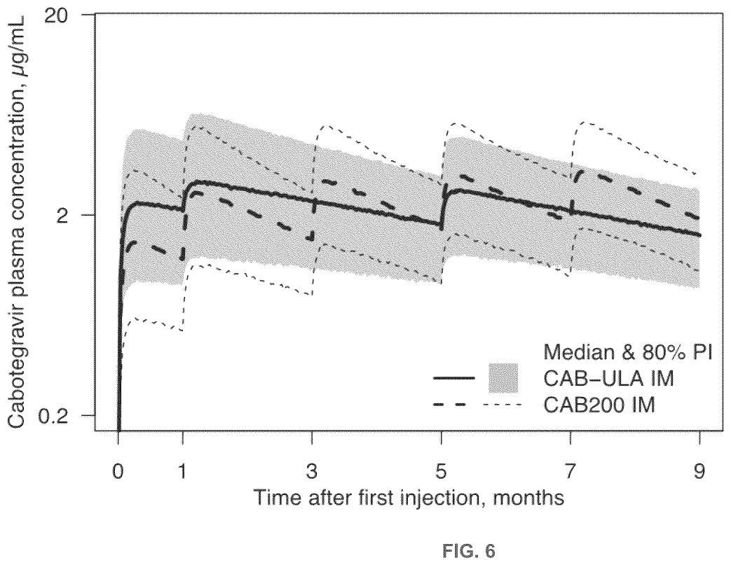

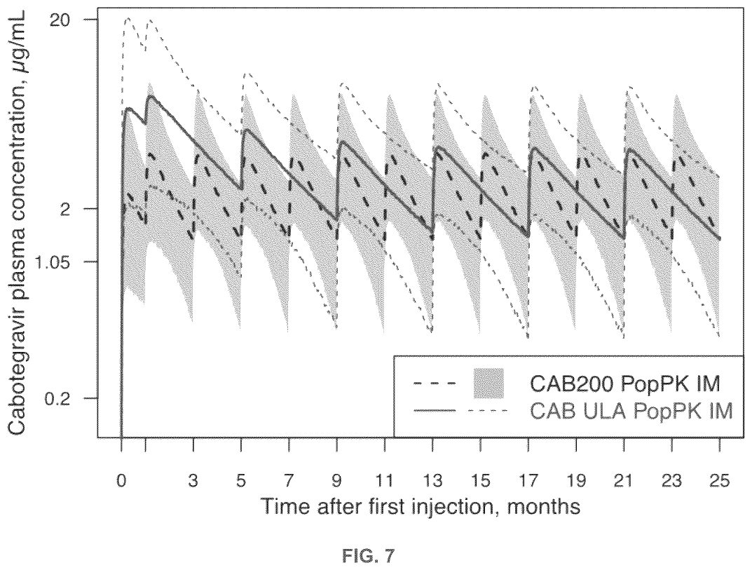

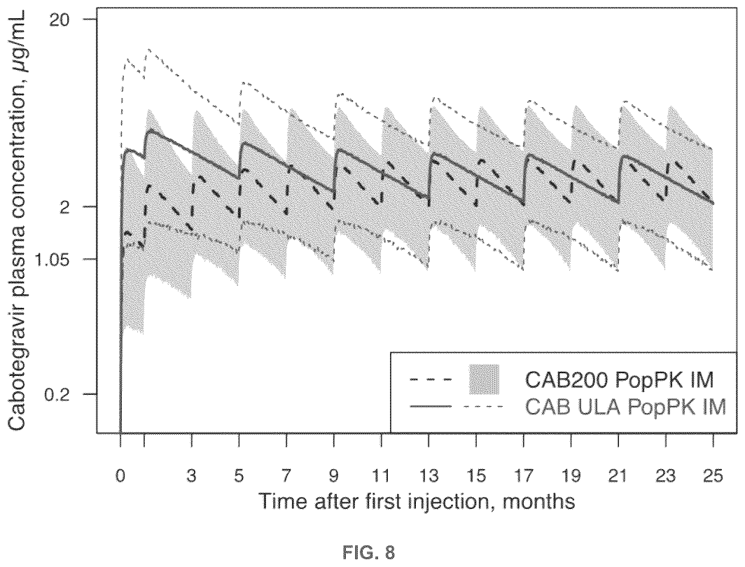

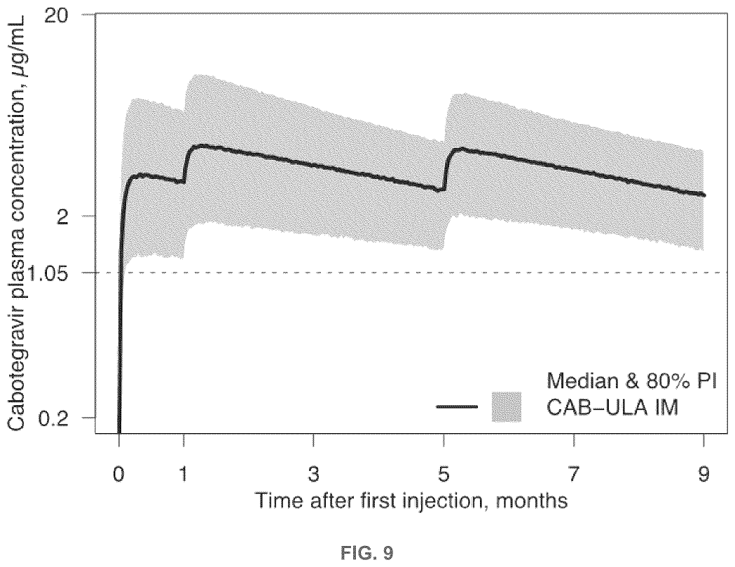

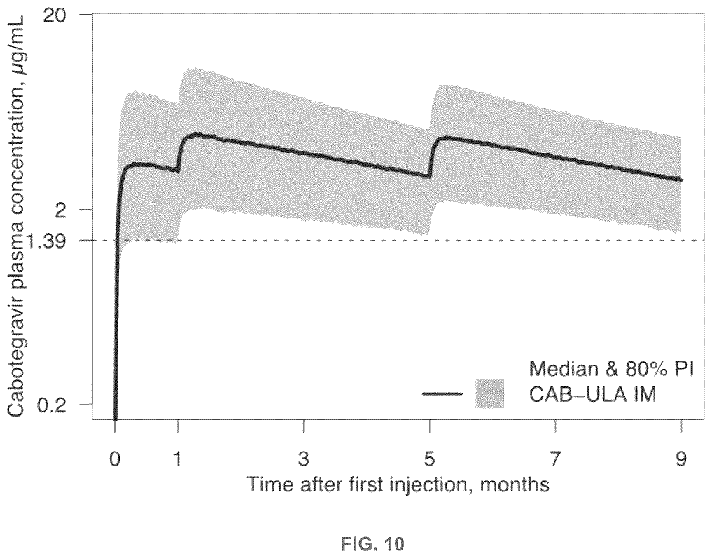

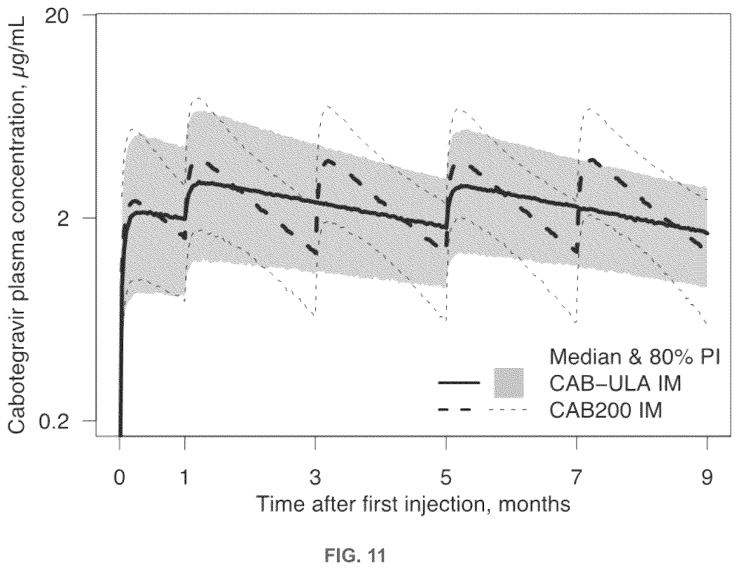

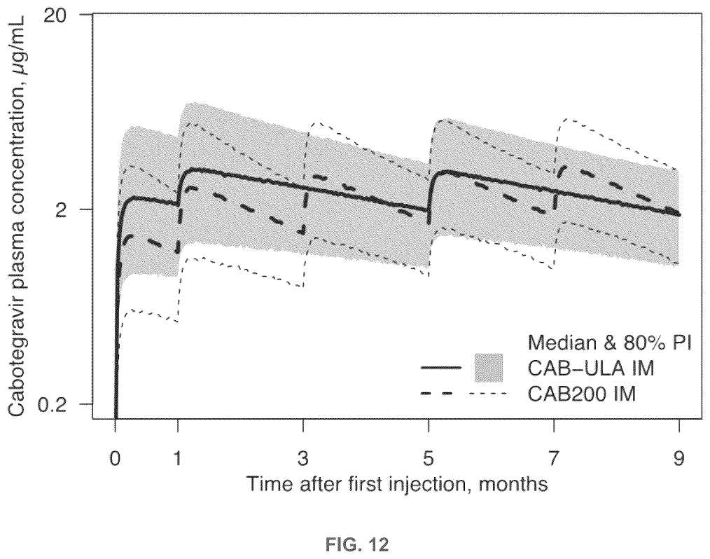

A cabotegravir population pharmacokinetic (PopPK) model was built based on PK data following cabotegravir (CAB) 200 mg/mL intramuscular (IM) injections collected in 16 historical studies (Population pharmacokinetics of cabotegravir following administration of oral tablet and long-acting intramuscular injection in adult HIV-1-infected and uninfected subjects. Han K, Baker M, Lovern M, Paul P, Xiong Y, Patel P, Moore K P, Seal C S, Cutrell A G, D'Amico R D, Benn P D, Landovitz R J, Marzinke M A, Spreen W R, Ford S L. Br J Clin Pharmacol. 2022 October; 88(10):4607-4622. doi: 10.1111/bcp.15439. Epub 2022 Jul. 4. PMID: 35695476) and updated based on PK data from 19 studies: LAI116585, LAI117010, LAI117011, LAI117020, 201741, 201479, 201480, 205696, LAI116482 (LATTE), LAI115428, LAI116815, 200056 (LATTE-2), 201120 (ECLAIR), 201103 (HPTN077), 201584 (FLAIR), 201585 (ATLAS), 207966 (ATLAS-2M), 201738 (HPTN 083) and 201739 (HPTN 084). A total of 34,850 CAB plasma concentrations collected at various time points from 2,694 participants were used to build this PopPK model. It was discovered that:
Long-acting intramuscular gluteal absorption rate constant (KA2) was 46.5% lower in people assigned female at birth than people assigned male at birth.
KA2 was 50.8% higher if the injection was split in people assigned male at birth, but KA2 was not associated with split injections in people assigned female at birth.
KA2 decreased with increasing BMI, and decreased more in people assigned male at birth than people assigned female at birth for the same amount of BMI increase.
KA2 was lower with longer needle length.
KA2 was 22.8% higher in study 201120 (ECLAIR) than other studies after adjusting for all covariates.
Apparent central clearance (CL/F) was 16.4% higher in current smokers.
   Based on the observed PK data from the study discussed in Example 9, this PopPK model was modified by setting the typical value of half-life (t_{1/2}) to 25 weeks for 533 mg/mL formulations and twice that if APRETUDE® for 400 mg/mL formulations to perform simulations for the cabotegravir formulations used in Example 9. It was assumed that:
t ½ following IM gluteal injections of the formulations in Example 9 with a concentration of 533 mg/mL of cabotegravir is 25 weeks,
t ½ following IM gluteal injections of the formulations in Example 9 for formulations with a concentration of 400 mg/mL is twice the t ½ of APRETUDE®,
effects of body weight, BMI and smoking status on PK, all variabilities (e.g., inter-individual variability and residual variability), and bioavailability are the same as for the 200 mg/mL IM gluteal injections,
PK following split IM gluteal injections of the formulations in Example 9 on both sides of the body is identical to PK following a single IM gluteal injection of the formulations in Example 9.
   The simulations were performed as follows: covariates including body weight, BMI and smoking status were resampled 5000 times from the distribution of these covariates for males and females, generating 5000 virtual male subjects and 5000 virtual female subjects. Individual PK parameters of the virtual subjects were calculated using subject-specific covariates and using the population parameter estimates, subject-specific NONMEM inter-individual errors (ETAs) sampled from the distributions that are decided by the estimated variance-covariate matrix of between-subject variability from the final population PK model. Concentration-versus-time profiles of the virtual subjects were calculated using the individual PK parameters. Residual variability (EPS) was included in the simulation. Median and 10th and 90th percentiles (80% prediction interval) of the simulated concentration-versus-time profiles of the virtual males and females were calculated.
   An adequate dose was defined as the dose that would meet these criteria:
1. maintain the median and 10th percentile of trough CAB plasma concentrations (Ctau) higher than those from the approved Q2M regimen of 200 mg/mL CAB IM gluteal injections in both people assigned male at birth and people assigned female at birth or
2. maintain Ctau higher than the sex-specific PrEP benchmarks in more than 90% of participants. The sex-specific PrEP benchmarks are the 10th percentile of Ctau observed in the Phase 3 studies 201738 (HPTN 083) for people assigned male at birth (1.05 μg/mL) and 201739 (HPTN 084) for people assigned female at birth (1.39 μg/mL).
   The simulations show that a Q4M regimen of IM gluteal injections with a maintenance dose of 1.5 mL of 533 mg/mL of cabotegravir (799.5 mg cabotegravir) (FIGS. 5 and 6) or 3 mL of 400 mg/mL cabotegravir (1200 mg cabotegravir) (FIGS. 7 and 8) would not maintain CAB plasma concentrations higher than the approved Q2M regimen of CAB 200 mg/mL IM gluteal injections in people assigned male or female at birth.
   A Q4M regimen of IM gluteal injections with a loading dose of at least 2132 mg (4 mL) and a maintenance dose of at least 1066 mg (2 mL) administered Q4M starting 1 month after the loading dose would meet criterion 1 i.e. would maintain CAB plasma concentrations higher than the approved Q2M regimen of CAB200 IM gluteal injections in both people assigned male at birth (FIG. 11) and people assigned female at birth (FIG. 12).
   A Q4M regimen of IM gluteal injections with a loading dose of at least 3200 mg (3 mL+3 mL) and a maintenance dose of at least 1600 mg (3 mL) that is administered Q4M starting at 1 month after the loading dose would meet both criteria:
maintain CAB plasma concentrations higher than the approved Q2M regimen of CAB200 IM gluteal injections in both people assigned male at birth (FIG. 3) and people assigned female at birth (FIG. 4).
maintain Ctau higher than 1.05 μg/mL and 1.39 μg/mL in more than 90% of people assigned male at birth (FIG. 9) and people assigned female at birth (FIG. 10), respectively. As shown in FIGS. 9 and 10, the lower boundary of the grey band (simulated 10th percentile of CAB-ULA) is higher than the horizontal dashed line (benchmark).

### Example 11

In Vivo Rat Intramuscular Pharmacokinetic Study for Compositions with Different Drug Concentration

The pharmacokinetics of two nanosuspension formulations at different cabotegravir concentrations (drug load) were assessed in Sprague Dawley male rats from intramuscular dosing. Briefly, two nanosuspension formulations of Cabotegravir at approximately 200 mg/mL (group 1) and 400 mg/mL (group 2), also differing in excipient composition, were administered intramuscularly by a single injection at target dose of 10 mg/Kg in each rat. Details of the formulations and particle size distributions are reported in table 21.

TABLE 21
Suspension formulations details for test articles
dosed in animal PK study Example 11.

| | Cabotegravir | Particle size | | | |
|---|---|---|---|---|---|
| | Concentra- | distribution | | | |
| | tion | (μm) | | | |
| Group | (mg/mL) | x10 | x50 | x90 | Formulation composition |
| 1 | 195 | 0.07 | 0.17 | 0.51 | 1.8% Polysorbate 20, 1.8% |
| | | | | | Polyethylene Glycol 3350, |
| | | | | | 3.1% Mannitol |
| 2 | 399 | 0.05 | 0.17 | 0.61 | 3.0% Polysorbate 20, 3.0% |
| | | | | | Polyethylene Glycol 3350 |
| | | | | | and 1.9% Mannitol |

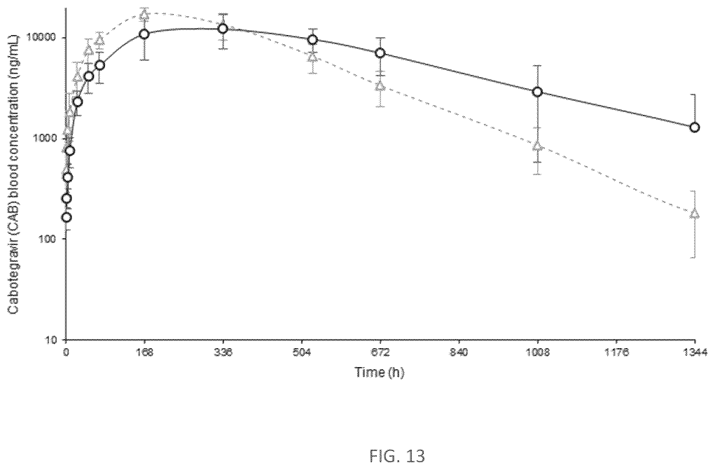

FIG. 13 shows the cabotegravir mean blood concentration time course for PK study Example 11 in semi-logarithmic scale. Nanosuspension formulation at approximately 400 mg/mL from Group 2 (t_{1/2}=224±47 h) has a significantly extended apparent terminal half-life (1.4-fold), compared to the nanosuspension at similar particle size distribution but lower cabotegravir concentration (200 mg/mL) from Group 1 (t_{1/2}=162±18 h). CAB200 nanosuspension formulation (200 mg/mL cabotegravir) is shown in the dashed line with triangles, and CAB400 nanosuspension formulation (400 mg/mL cabotegravir) is shown in the continuous line with circles. Data points represent mean±standard deviation.

Preclinical data from Example 11 (see FIG. 13) underscore the positive effect of higher cabotegravir concentration in the formulation on apparent terminal t_{1/2}. This effect of concentration is leveraged in the lyophilized presentation to further extend the duration above target that can be achieved with a micron particle size distribution. This effect allows to achieve longer duration of action for cabotegravir, without requiring a significantly larger particle size, which could lead to issues with syringeability (Samaradi et al.), such as needle clogging.

In addition, higher cabotegravir concentration in the drug product allows for the administration of a lower injection volume for the same dosing, which is preferable for the patient.

All of the U.S. patents, U.S. patent application publications, U.S. patent applications, foreign patents, foreign patent applications, and non-patent publications referred to in this specification are incorporated herein by reference, in their entirety to the extent not inconsistent with the present description.

From the foregoing it will be appreciated that, although specific embodiments have been described herein for purposes of illustration, various modifications may be made without deviating from the spirit and scope of the disclosure. Accordingly, the disclosure is not limited except as by the appended claims.

1. A pharmaceutical composition comprising:
cabotegravir;
a wetting agent, wherein the wetting agent is polysorbate 80 (PS80);
a stabilizer, wherein the stabilizer is sodium carboxymethylcellulose (CMC); and
a tonicity adjuster, wherein the tonicity adjuster is mannitol;
wherein cabotegravir is present in the form of particles having a mass median diameter (X50) of between (and including) 2.5 μm and 10 μm.

2. The pharmaceutical composition according to claim 1, wherein the cabotegravir particles have an X50 value greater than or equal to 3 μm and less than or equal to 8.5 μm.

3. The pharmaceutical composition according to claim 1, wherein the cabotegravir particles have an X50 value greater than or equal to 3.5 μm and less than or equal to 8.0 μm, and wherein the cabotegravir particles have an X90 value greater than or equal to 7.0 μm and less than or equal to 18.0 μm.

4. The pharmaceutical composition according to claim 1, wherein cabotegravir is present in an amount of from about 350 mg to about 1650 mg.

5. The pharmaceutical composition according to claim 1, wherein cabotegravir is present in an amount of about 800 mg.

6. The pharmaceutical composition according to claim 1, wherein cabotegravir is present in an amount of about 1600 mg.

7. The pharmaceutical composition according to claim 1,
wherein a weight ratio of the wetting agent to cabotegravir is in a range of from about 1:100 to about 1:150;
wherein a weight ratio of the stabilizer to cabotegravir is in a range of from about 1:70 to about 1:120; and
wherein a weight ratio of the tonicity adjuster to cabotegravir is in a range of from about 1:8 to about 1:25.

8. The pharmaceutical composition according to claim 1, wherein a weight ratio of cabotegravir:PS80:sodium CMC:mannitol is about 100:1:1.25:8.75.

9. The pharmaceutical composition according to claim 1, wherein a weight ratio of cabotegravir:PS80:sodium CMC:mannitol is about 400:3:3.7:25.9.

10. The pharmaceutical composition according to claim 1, wherein the pharmaceutical composition is provided as a lyophilized powder.

11. The pharmaceutical composition according to claim 1, wherein the concentration of cabotegravir is in a range of from about 300 mg/mL to about 650 mg/mL.

12. The pharmaceutical composition according to claim 1, wherein the concentration of cabotegravir is about 400 mg/mL.

13. The pharmaceutical composition according to claim 1, wherein the concentration of cabotegravir is about 533 mg/mL.

14. The pharmaceutical composition according to claim 1, wherein the pharmaceutical composition is
(a) reconstituted from a lyophilized powder with a suitable liquid, or
(b) provided as a liquid composition.

15. The pharmaceutical composition according to claim 14, wherein the suitable liquid is water.

16. A method for treating HIV in a human in need thereof comprising administering to said human a therapeutically effective amount of the pharmaceutical composition according to claim 1.

17. The method according to claim 16, wherein the method comprises a step of administering the pharmaceutical composition subcutaneously or intramuscularly.

18. The method according to claim 17, wherein about 300 mg to about 3200 mg of cabotegravir is administered to the human.

19. The method according to claim 17, wherein the method comprises administering the pharmaceutical composition to the human once every month, once every 2 months, once every 3 months, once every 4 months, once every 5 months, or once every 6 months.

20. The method according to claim 17, wherein the method comprises administering the pharmaceutical composition in combination with one or more nucleoside reverse transcriptase inhibitors (NRTTIs), one or more non-nucleoside reverse transcriptase inhibitors (NNRTI), one or more capsid inhibitors, and/or one or more broadly neutralizing antibodies (bnAbs).

21. A method for preventing HIV in a human comprising administering to said human an effective amount of the pharmaceutical composition according to claim 1.

22. The method according to claim 21, wherein the method comprises a step of administering the pharmaceutical composition subcutaneously or intramuscularly.

23. The method according to claim 22, wherein about 300 mg to about 3200 mg of cabotegravir is administered to the human.

24. The method according to claim 22, wherein the method comprises administering the pharmaceutical composition to the human once every month, once every 2 months, once every 3 months, once every 4 months, once every 5 months, or once every 6 months.

25. The method according to claim 22, wherein the method comprises administering the pharmaceutical composition to the human once every 4 months.

26. A kit comprising a container that contains the pharmaceutical composition according to claim 1 as a lyophilized powder.

27. The kit according to claim 26, wherein the kit further comprises a container comprising water.

28. A method of preparing a reconstituted solution, the method comprising providing the kit of claim 26 and contacting the lyophilized composition with a suitable liquid to produce a reconstituted solution.

29. The method according to claim 28, wherein the suitable liquid is water.


SEQUENCE LISTING (8 sequences)

SEQ ID NO: 1 - protein, 5 aa, synthetic construct
AHILF

SEQ ID NO: 2 - protein, 17 aa, synthetic construct
WIKPQYGAVNFGGGFRD

SEQ ID NO: 3 - protein, 13 aa, synthetic construct
DRSYGDSSWALDA

SEQ ID NO: 4 - protein, 11 aa, synthetic construct
QTSQGVGSDLH

SEQ ID NO: 5 - protein, 7 aa, synthetic construct
HTSSVED

SEQ ID NO: 6 - protein, 5 aa, synthetic construct
QVLQF

SEQ ID NO: 7 - protein, 122 aa, synthetic construct [sequence omitted]

SEQ ID NO: 8 - protein, 103 aa, synthetic construct [sequence omitted]